(19) United States
(12) Patent Application Publication
(10) Pub. No.: US 2011/0286931 A1
(43) Pub. Date: Nov. 24, 2011

(54) CARDIAC ARRHYTHMIA TREATMENT METHODS AND BIOLOGICAL PACEMAKER

(75) Inventor: Eduardo Marban, Beverly Hills, CA (US)

(73) Assignee: THE JOHNS HOPKINS UNIVERSITY, Baltimore, MD (US)

(21) Appl. No.: 13/167,141

(22) Filed: Jun. 23, 2011

Related U.S. Application Data
(62) Division of application No. 12/035,077, filed on Feb. 21, 2008.
Application No. 12/035,077 is a continuation-in-part of application No. 11/508,957, filed on Aug. 24, 2006.
Application No. 11/508,957 is a continuation of application No. 10/855,989, filed on May 28, 2004, now abandoned.
Application No. 10/855,989 is a division of application No. 09/947,953, filed on Sep. 6, 2001, now Pat. No. 7,034,008.
Application No. 12/035,077 is a continuation-in-part of application No. 10/476,259, filed on Aug. 10, 2004.
(60) Provisional application No. 60/230,311, filed on Sep. 6, 2000.
(60) Provisional application No. 60/295,889, filed on Jun. 5, 2001.
(60) Provisional application No. 60/287,088, filed on Apr. 27, 2001.

(51) Int. Cl. C12Q 1/02; A61P 9/06; C12N 15/63; A61K 49/00

(52) U.S. Cl. 424/9.2; 435/29; 435/320.1

(57) ABSTRACT

Disclosed are methods of preventing or treating cardiac arrhythmia. In one embodiment, the methods include administering to an amount of at least one polynucleotide that modulates an electrical property of the heart. The methods have a wide variety of important uses including treating cardiac arrhythmia. Also disclosed are methods and systems for modulating electrical behavior of cardiac cells. Preferred methods include administering a polynucleotide or cell-based composition that can modulate cardiac contraction to desired levels, e.g., the administered composition functions as a biological pacemaker.

### CROSS-REFERENCE TO RELATED APPLICATIONS

The present application is a divisional of U.S. application Ser. No. 12/035,077 filed Feb. 21, 2008, which is a continuation-in-part of U.S. application Ser. No. 11/508,957, filed Aug. 24, 2006, which is a continuation of U.S. application Ser. No. 10/855,989, filed on May 28, 2004, now abandoned, which is a divisional of U.S. application Ser. No. 09/947,953, filed on Sep. 6, 2001, now U.S. Pat. No. 7,034,008, which claims priority to U.S. Provisional Application No. 60/230,311, filed on Sep. 6, 2000, and U.S. Provisional Application No. 60/295,889, filed on Jun. 5, 2001; and wherein the 12/035,077 application is also a continuation-in-part of U.S. application Ser. No. 10/476,259, filed Aug. 10, 2004, which is a national stage entry of PCT/US02/13671, filed Apr. 29, 2002, which claims priority to U.S. Provisional Application No. 60/287,088, filed on Apr. 27, 2001, the disclosures of which are incorporated herein by reference in their entireties.

### STATEMENT REGARDING FEDERALLY SPONSORED R&D

Funding for embodiments of the present invention was provided in part by the Government of the United States by virtue of Grant No. NIH P50 HL52307 by the National Institutes of Health. Thus, the Government of the United States has certain rights in and to embodiments of the invention claimed herein.

### REFERENCE TO SEQUENCE LISTING, TABLE, OR COMPUTER PROGRAM LISTING

All disclosed sequences are listed on the attached Sequence Listing which forms part of this specification.

### BACKGROUND OF THE INVENTION

1. Field of the Invention

Embodiments of the invention relate generally to methods for the prevention or treatment of heart arrhythmia and methods to provide and/or modulate a cardiac pacemaker function. Preferred methods generally involve administering at least one therapeutic polynucleotide to a mammal sufficient to modulate at least one electrical property of the heart. Modulation of the electrical property addresses the arrhythmia typically by encouraging normal heart electrical function. Preferred embodiments of genetically-engineered pacemakers can be employed as an alternative or supplement to implantable electronic pacemakers to induce or modulate ventricular or atrial firing rate.

2. Description of the Related Art

The mammalian heart is understood to maintain an intrinsic rhythm by creating electric stimuli. Generally, the stimuli form a depolarization wave that originates in so-called pacemakers and then propagates within specialized cardiac conducting tissue and the myocardium. The usually well-ordered wave movement facilitates coordinated contractions of the myocardium. These contractions are the engine that moves blood throughout the body. See generally The Heart and Cardiovascular System. Scientific Foundations. (1986) (Fozzard, H. A. et al. eds) Raven Press, NY, herein incorporated by reference.

Under most circumstances, cardiac stimuli are controlled by recognized physiological mechanisms. However there has been long-standing recognition that abnormalities of excitable cardiac tissue can lead to abnormalities of the heart rhythm. These abnormalities are generally referred to as arrhythmias. Most arrhythmias are believed to stem from defects in cardiac impulse generation or propagation that can substantially compromise homeostasis, leading to substantial patient discomfort or even death. For example, cardiac arrhythmias that cause the heart to beat too slowly are known as bradycardia, or bradyarrhythmia, which result in greater than 255,000 electronic pacemaker implants per year in the United States. In contrast, arrhythmias that cause the heart to beat too fast are referred to as tachycardia, or tachyarrhythmia. See generally Cardiovascular Arrhythmias (1973) (Dreifus, L. S, and Likoff, W. eds) Grune & Stratton, NY, herein incorporated by reference.

The significance of these and related heart disorders to public health cannot be exaggerated. Symptoms related to arrhythmias range from nuisance, extra heart beats, to life-threatening loss of consciousness. Complete circulatory collapse has also been reported. Morbidity and mortality from such problems continues to be substantial. In the United States alone for example, cardiac arrest accounts for 220,000 deaths per year. There is thought to be more than 10% of total American deaths. Atrial fibrillation, a specific form of cardiac arrhythmia, impacts more than 2 million people in the United States. Other arrhythmias account for thousands of emergency room visits and hospital admissions each year. See e.g., Bosch, R. et al. (1999) in Cardiovas Res. 44: 121, herein incorporated by reference, and references cited therein.

Cardiac electrophysiology has been the subject of intense interest. Generally, the cellular basis for all cardiac electrical activity is the action potential (AP). The AP is conventionally divided into five phases in which each phase is defined by the cellular membrane potential and the activity of potassium, chloride, and calcium ion channel proteins that affect that potential. Propagation of the AP throughout the heart is thought to involve gap junctions. See Tomaselli, G. and Marban, E. (1999) in Cardiovasc. Res. 42: 270, herein incorporated by reference, and references cited therein.

There have been limited attempts to treat cardiac arrhythmias and related heart disorders. Specifically, many of the past attempts have been confined to pharmacotherapy, radiofrequency ablation, use of implantable devices, and related approaches. Unfortunately, this has limited options for successful patient management and rehabilitation.

In particular, radiofrequency ablation has been reported to address a limited number of arrhythmias e.g., atrioventricular (AV) node reentry tachycardia, accessory pathway-mediated tachycardia, and atrial flutter. However, more problematic arrhythmias such as atrial fibrillation and infarct-related ventricular tachycardia, are less amenable to this and related therapies. Device-based therapies (pacemakers and defibrillators, for instance) have been reported to be helpful for some patients with bradyarrhythmias and lifesaving for patients with tachyarrhythmias. However, such therapies does not always prevent tachyarrhythmias. Moreover, use of such implementations is most often associated with a prolonged commitment to repeated procedures, significant expense, and potentially catastrophic complications including infection, cardiac perforation, and lead failure.

Drug therapy remains a popular route for reducing some arrhythmic events. However, there has been recognition that systemic effects are often poorly tolerated. Moreover, there is belief that proarrhythmic tendencies exhibited by many drugs may increase mortality in many situations. See generally Bigger, J. T and Hoffman, B. F. (1993) in The Pharmacological Basis of Therapeutics 8th Ed. (Gilman, A. G et al. eds) McGraw-Hill, NY, herein incorporated by reference, and references cited therein.

It would be desirable to have more effective methods for treating or preventing cardiac arrhythmias. It would be especially desirable to have gene therapy methods for treating or preventing such arrhythmias. It would also be desirable to have new methods to provide a desired rate of cardiac contraction (firing rate).

### SUMMARY OF THE INVENTION

Several embodiments of the present invention provides methods of preventing or treating cardiac arrhythmia in a mammal. In general, the methods involve administering to the mammal at least one polynucleotide that preferably modulates at least one electrical property of the heart. Use of the polynucleotides according to embodiments of the invention modulates the heart electrical property, thereby preventing or treating the cardiac arrhythmia.

There has been a long-felt need for more effective anti-arrhythmic therapies. Several embodiments of the invention address this need by providing, for the first time, therapeutic methods for administering one or more therapeutic polynucleotides to the heart under conditions sufficient to modulate (increase or decrease) at least one heart electrical property. Preferred use of several embodiments of the invention modulates heart electrical conduction preferably reconfigures all or part of the cardiac action potential (AP). That use helps achieve a desired therapeutic outcome. Significant disruption of normal electrical function is usually reduced and often avoided by the present methods. Moreover, use of several embodiments of the invention is flexible and provides, also for the first time, important anti-arrhythmic strategies that can be tailored to the health requirements of one patient or several as needed.

Several embodiments of the invention provide other advantages that have been heretobefore difficult or impossible to achieve. For example, and unlike prior practice, several embodiments of the invention are genetically and spatially controllable (e.g., they provide for administration of at least one pre-defined polynucleotide to an identified heart tissue or focal area). Since there is recognition that many protein encoding polynucleotides can be expressed successfully in heart tissue, several embodiments of the invention are a generally applicable anti-arrhythmia therapy that can be employed to supply the heart with one or a combination of different therapeutic proteins encoded by the polynucleotides. Such proteins can be provided transiently or more long-term as needed to address a particular cardiac indication.

Several embodiments of the invention provide further benefits and advantages. For example, practice of prior anti-arrhythmic approaches involving pharmacotherapy, radiofrequency ablation, and implantable device approaches is reduced and oftentimes eliminated by several embodiments of the invention. Moreover, several embodiments of the invention provide highly localized gene delivery. Importantly, treated cells and tissue usually remain responsive to endogenous nerves and hormones in most cases. In particular, several embodiments of the invention, relating to localized coronary circulation, provide targeted delivery to isolated regions of the heart. In some embodiments, proximity to endocardium allows access by intracardiac injection. Therapeutic effects are often readily detected e.g., by use of standard electrophysiological assays as provided herein. Also importantly, many gene transfer-induced changes in accord with several embodiments of the present invention can be rescued, if needed, by conventional electrophysiological methods.

In addition, we now provide gene transfer and cell administration methods that can create a pacemaker function, and/or modulate the activity of an endogenous or induced cardiac pacemaker function.

Methods of several embodiments of the invention may be employed to create and/or modulate the activity of an endogenous pacemaker (such as the sinotrial node of a mammalian heart) and/or an induced pacemaker (e.g. biological pacemaker generated from stem cells or converted electrically-quiescent cells).

In particular, in one embodiment a method of assaying whether an agent affects heart rate is provided. The method involves contacting a cardiac cell of a heart with an effective amount of a compound to cause a repetitive or sustainable heart rate, and then measuring the heart rate. The method further involves providing the heart with an agent to be assayed for its affects on heart rate, and again measuring the heart rate. The difference between the heart rates is compared, thereby determining whether the agent affects heart rate.

In some embodiments of the method, the heart is mammalian. In some embodiments, the cardiac cell is a cardiac myocyte. In some embodiments, the compound is a nucleic acid encoding an HCN channel. In some embodiments, the HCN channel is HCN1 or HCN2.

In some embodiments, the step of contacting can involve topical application, injection, electroporation, microinjection liposome application, viral-mediated contact, contacting the cell with the nucleic acid, and coculturing the cell with the nucleic acid. Administration of contacting can involve topical administration, adenovirus infection, viral-mediated infection, microinjection, electroporation, liposome-mediated transfer, topical application to the cell, and catheterization.

In another embodiment, a method of assaying whether an agent affects heart rate is provided. The method involves isolating or disaggregating cardiac myocytes from a heart and measuring the beating rate of the cardiac myocytes after isolation. The method further involves contacting a set of the cardiac myocytes with an agent to be assayed for its effects on heart rate and then measuring the heart rate. The two measurements can be compared, thereby determining whether the agent affects heart rate. In some embodiments, the measuring steps are performed using a patch clamp, or other methods known to those in the art (such as calcium sensitive dyes and photodiodes).

In addition to assaying whether an agent affects heart rate, the affect on membrane potential, cell activation, cell contraction can also be determined by methods analogous to those described above. Methods according to embodiments of the invention can be performed in vitro or in situ.

In some embodiments, a vector which includes a compound that encodes an ion channel gene is provided. The vector can be a virus, a plasmid, a cosmid or an adenovirus. The compound can be a nucleic acid which encodes an HCN channel such as HCN1 or HCN2, or a combination of isoforms thereof (e.g., either co-expressed or formed into a single construct or chimeric).

### BRIEF DESCRIPTION OF THE DRAWINGS

FIGS. 1A-B are photographs showing gene transfer to the AV node after exposure to Adβgal. FIGS. 1C-D are photographs showing gene transfer to various non-target organ tissue.

FIG. 2A is a graph showing reduction in heart rate during atrial fibrillation after gene transfer of inhibitory G subunit (Gᵢ₂) FIG. 2B is a related electrocardiogram.

FIG. 3A is a graph showing reduction in heart rate during atrial fibrillation after gene transfer of inhibitory G subunit (Gᵢ₂) and infusion of epinephrine. FIG. 3B is a related electrocardiogram.

FIG. 4A is a Western blot of AV nodal tissue showing Gᵢ₂ over expression. FIG. 4B is a graph showing heart rate following gene transfer.

FIG. 5A is a graph showing comparison of Iₖᵣ current in presence and absence of gene transfer-mediated overexpression of HERG. FIG. 5B is a photograph showing related action potential (AP).

FIG. 6 is a drawing showing changes in atrial action potential after prolonged atrial fibrillation. The dotted line indicates a normal atrial action potential morphology.

FIG. 7A is a graph showing comparison of Iₖᵣ current in presence and absence of gene transfer-mediated overexpression of dominant-negative mutant of HERG. FIG. 7B is a photograph showing related action potential (AP) of the mutant HERG.

FIGS. 8A and 8B depict a preferred therapeutic agent delivery device (intravascular injection catheter) of several embodiments of the invention. FIG. 8B shows the indicated area of device in expanded cross-section.

FIG. 9A is a drawing showing the amino acid sequence of the human Gαᵢ₂ sequence (SEQ ID NO: ID NO: 10)(NCBI protein sequence no PO4899).

FIGS. 9B-C are drawings showing the nucleic acid sequence encoding the human Gαᵢ₂ sequence shown in FIG. 9A. FIGS. 9B-C show the nucleic acid sequences (SEQ ID NOs: 1-9, respectively in order of appearance) in exon form.

FIGS. 10A-B are graphs showing action potentials in guinea pig ventricular myocytes expressing Kir2.1AAA.

FIG. 11 shows an assessment of gene transfer efficacy. X-gal staining of microscopic sections of left ventricle (LV) 48 hours after injection of AdCMV-gal into the LV cavity was used to assess transduction efficacy. Transduced cells (stained blue) were observed throughout the LV wall. This gene delivery method achieved transduction of 20% of ventricular myocytes without obvious cell damage.

FIG. 12 shows specificity of I_{K1} suppression. (A,B,C) The average current density of I_{K1} was significantly reduced in Kir2.1AAA-transduced cells (n=9) compared with control cells (n=7, P<0.0001). (D,E,F). Further showing that the results are primarily due to the specific effects of modulating functional Kir2.1 channel number, the L-type calcium current was not altered in Kir2.1-AAA transduced myocytes (−4.2.+−.0.9 pA/pF, n=4) compared to nontransduced cells (−4.5.+−.0.2 pA/pF, n=6).

FIG. 13 shows that action potential phenotype is determined by I_{K1} density. (A) Stable APs are evoked by depolarizing external stimuli in control ventricular myocytes with a robust I_{K1} (B, recorded at −50 mV). In Kir2.1AAA-transduced myocytes with moderately depressed I_{K1} (D), APs with a long QT phenotype were evoked (C). Spontaneous APs (E) were observed in Kir2.1AAA cells with severely depressed I_{K1} density (F). Three distinct ranges of I_{K1} density (G) were recognized. Myocytes in which IK1 was suppressed below 0.4 pA/pF exhibited a pacemaker phenotype.

FIG. 14 shows that the calcium current is the excitatory current underlying the spontaneous APs. Kir2.1AAA-transduced cells with a pacemaker phenotype were unaffected by the Na channel blocker tetrodotoxin (10 μM, A,B), but spontaneous firing ceased during exposure to calcium channel blockers (cadmium 200 μM, C,D; nifedipine 10 μM, E,F).

FIG. 15 shows that application of isoproterenol (1 μM) increased the frequency of spontaneous AP in four Kir2.1AAA-transduced myocytes exhibiting pacemaking activity (A,B). Average cycle length was reduced from 435.+−0.27 ms at baseline to 351.+−0.18 ms (n=4) during isoproterenol exposure (P<0.01) (C).

FIG. 16 shows electrocardiograms before and after gene delivery. (A) In 3 of 5 animals, QT intervals were prolonged 72 hours after gene transfer of Kir2.1AAA. (B) In 2 of 5 animals, ventricular rhythms developed. P waves (blue A and arrow) and wide QRS complexes (red V and arrow) march through to their own rhythm except a QRS complex inscribed with V which is a fusion beat. The baseline ECG recording for this animal was normal sinus rhythm (not shown, but similar to panel A).

FIGS. 17A-17C show putative transmembrane topology of HCN-encoded pacemaker channels. In FIG. 17.B: the six transmembrane segments (S1-S6) of a monomeric subunit of HCN1 channels are shown. The approximate location of the GYG signature motif is highlighted as shown. The cyclic nucleotide-binding domain (CNBD) is in the C-terminal region. In FIG. 17A: sequence comparison of the ascending limb of the S5-S6 P-loops of various HCN and depolarization activated (Kv) K⁺ channels (SEQ ID NO: 11-17, respectively). The GYG triplet (highlighted) is conserved in all K⁺-selective channels known except in rare occasions, such as that of the HERG K⁺ channels, whose middle position is occupied by the conservative aromatic variant phenylalanine instead of tyrosine. FIG. 17C compares the amino acid sequences (SEQ ID NO: 18-25, respectively) of the S3-S4 linker and S4 segment of HCN isoforms 114 with those of a hyperpolarization-activated sea urchin sperm channel (SPH1), a hyperpolarization-activated K⁺ channel cloned from the plant Arabidopsis thalin7a (KAT1), and depolarization-activated Shaker and HERG K⁺ channels. The S4 of HCN channels contains 9 basic amino acids regularly spaced from each other by two hydrophobic amino acids except at the sixth position, where a neutral serine is found in place of a cationic residue. SPIH and KAT1 channels have one fewer basic residue in their S4 segments compared to HCN channels, but again have a serine dividing the S4 into two portions. This S4 serine is not found in Kv channels; it divides the HCN voltage-sensing motif into two domains and has been hypothesized to be responsible for the unique hyperpolarization-activated opening of HCN channels.

FIG. 18 shows the effects of replacing GYG triplet in HCN1 with alanines (GYG₃₆₅₋₃₆₇AAA) on HCN1 currents. A) Representative traces of whole-cell currents recorded from oocytes injected with WT HCN1 and HCN1-AAA cRNA, and an uninjected oocyte as indicated. The electrophysiological protocol used to elicit currents is given in the inset. A family of 3-sec electrical pulses ranging from 0 to −150 mV in 10 mV increments was applied to oocytes from a holding potential of −30 mV. Tail currents were recorded at −140 mV. Whereas hyperpolarization-activated time-dependent currents were obvious from oocytes injected with WT HCN1, no measurable currents were observed from HCN1-AAA-injected and uninjected cells when the same protocol was used. B) Steady-state current-voltage relationships of WT HCN1- and HCN1-AAA-injected (solid squares and triangles, respectively), and uninjected (open circles) oocytes. Data shown are mean.+−.SEM.

FIG. 19 shows that HCN1 AAA suppressed the normal activity of WT HCN1 in a dominant-negative manner. A) Representative current tracings recorded from oocytes injected with 50 mL WT HCN1, 50 mL WT HCN1+50 mL dH₂O, and 50 mL WT HCN1+50 mL HCN1-AAA cRNA (concentration=1 ng/nL). The same voltage protocol from FIG. 18 was used. Co-injection of WT HCN1 and HCN1-AAA significantly suppressed normal channel activity. WT HCN1 tail currents (enclosed in a box) are magnified in D). B) Bar graph summarizing the averaged current magnitudes of each of the groups from A) measured at the end of a 3 second pulse to −140 mV from a holding potential of −30 mV normalized to that of 50 mL WT HCN alone. p<0.01. C) Steady-state current-voltage relationships of the same groups from A). D) Tail currents of WT HCN1 at −140 mV. Fitting these currents with a mono-exponential function allows estimation of the time constants for activation (cf. FIG. 21D).

FIG. 20 shows the dominant-negative effect of HCN1-AAA on WT-HCN1, and 2 with varied WT:AAA ratio.

Current suppression of WT HCN1 and HCN2 by HCN1-AAA plotted against the WT:AAA ratio of cRNA injected. Suppression of both HCN1 and HCN2 increased with decreasing WT:AAA ratio. Broken lines represent the suppression-ratio relationship statistically predicted from dimerization, trimerization, tetramerization and pentamerization of HCN monomers as indicated. The data also indicates that the endogenous HCN channel activity can be modulated by the AAA contruct disclosed herein.

FIG. 21 shows the dominant-negative suppressive effect of HCN1-AAA did not alter gating and permeation properties of HCN1 channels.

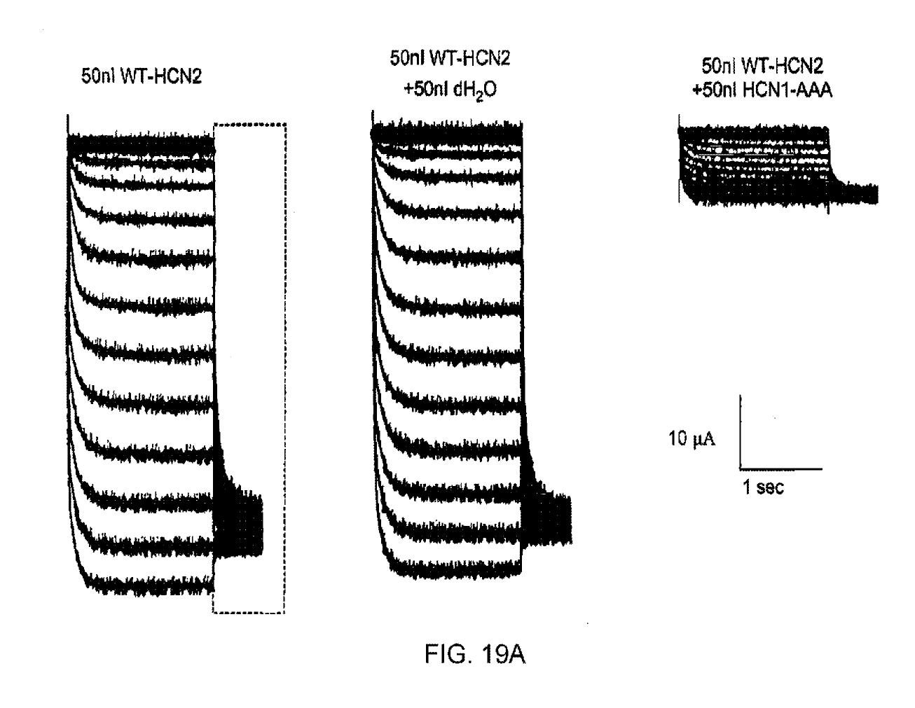

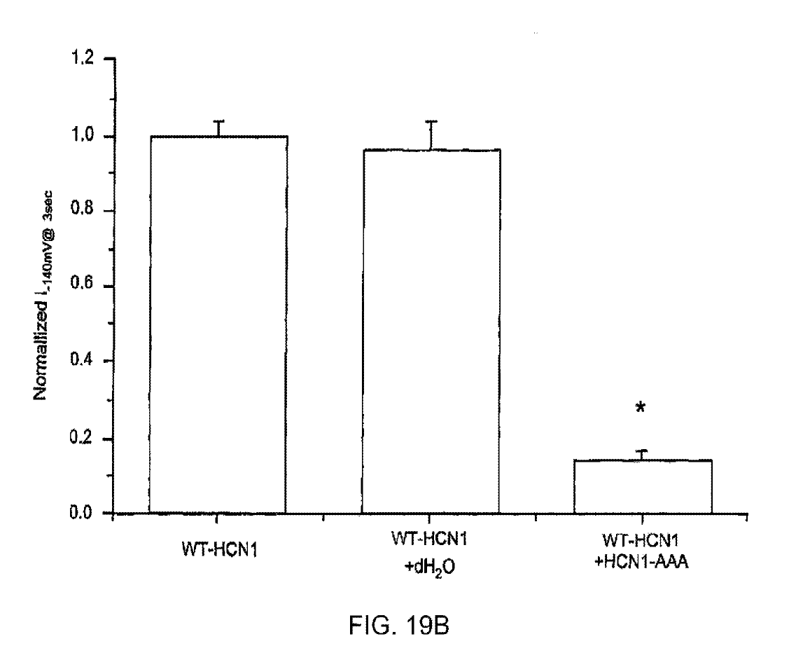

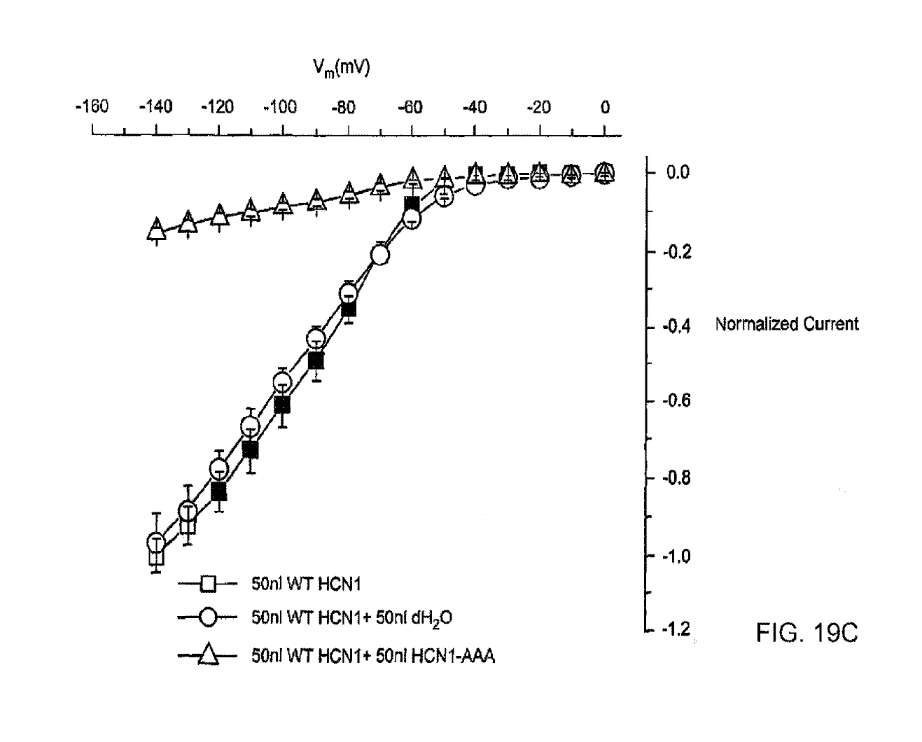

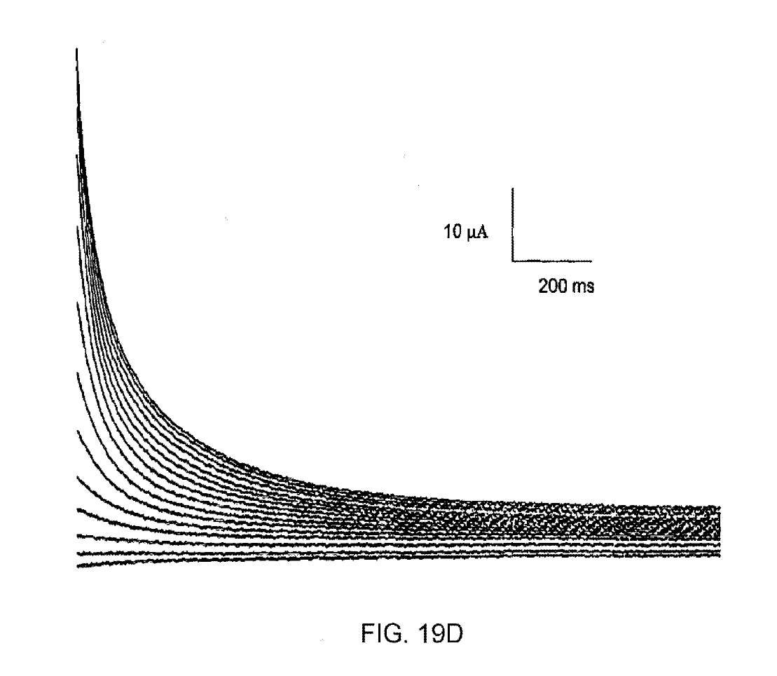

A) Steady-state activation curves of WT HCN1 alone and after suppression by HCN1AAA (ratio=1:1). Tail currents were measured immediately after pulsing to −140 mV using the same protocol as FIG. 19A (cf. inset), normalized to the largest tail recorded and plotted against the preceding prepulse potentials. Neither the mid-point nor the slope factor was different among the two groups.

B-C) Electrophysiological protocol used for obtaining tail current-voltage relationships by stepping membrane potentials from −100 to +40 mV with 10 mV increments after a 3 second prepulse to −140 mV. A representative family of tail currents recorded from an oocyte injected with 50 nL of 1 ng/nL WT HCN1 cRNA only is shown, and magnified as shown. Fitting these currents with a mono-exponential function allows estimation of the time constants for deactivation (τdeact).

D) Tail current-voltage relationships measured from oocytes injected with WT HCN1 alone or co-injected with WT HCN1 and HCN1-AAA (ratio=1:1). Whole-cell currents were suppressed by HCN1-AAA but the reversal potential was not changed. E) Summary Of ract (squares) and tact (circles) of currents induced by the injection of WT HCN1 alone (solid symbols) or by 1:1 co-injection of both WT HCN1 and HCN1-AAA (open symbols) Distribution of i was bell-shaped with mid-points similar to those derived from the corresponding steady-state activation curves. Gating kinetics of expressed currents were also not changed by HCN1-AAA co-injection.

FIG. 22 shows the effects of HCN1-AAA on HCN2 channels. (A) Representative current tracings recorded from oocytes injected with 50 nL WT HCN2, 50 nL WT HCN2+50 mL dH₂O and 50 nL WT HCN2+50 mL HCN1-AAA cRNA. HCN1-AAA also suppressed the activity of WT HCN2. (B) Current suppression at −140 mV of WT HCN2 by HCN 1-AAA plotted against the WT HCN2:HCN1-AAA ratio of cRNA injected. C. Steady-state current-voltage relationships of the same groups from A). Steady-state activation (D), reversal potential (E), and activation and deactivation kinetics (F) of WT HCN2 expressed alone and co-expression with HCN1-AAA (ratio=1:) were identical (p>0.05).

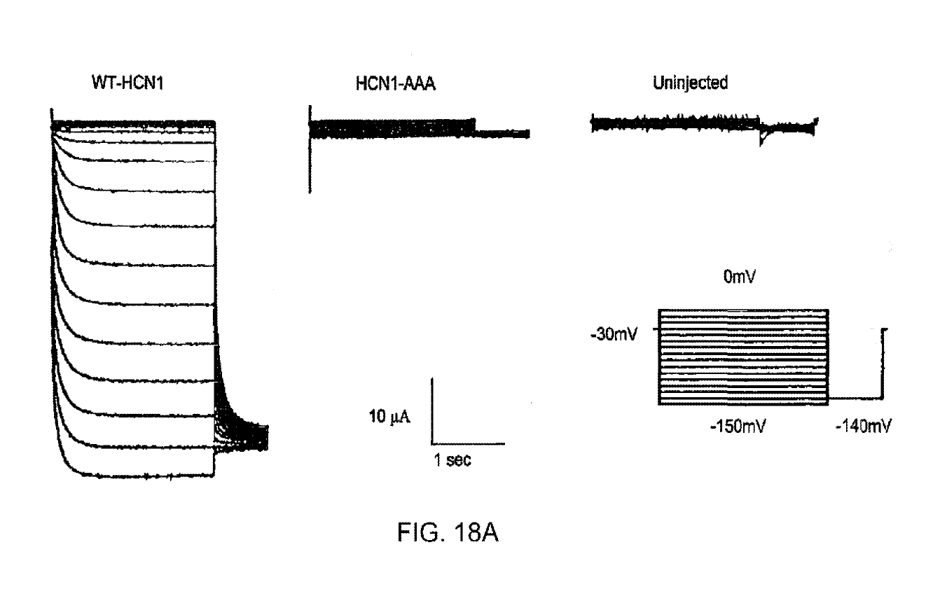

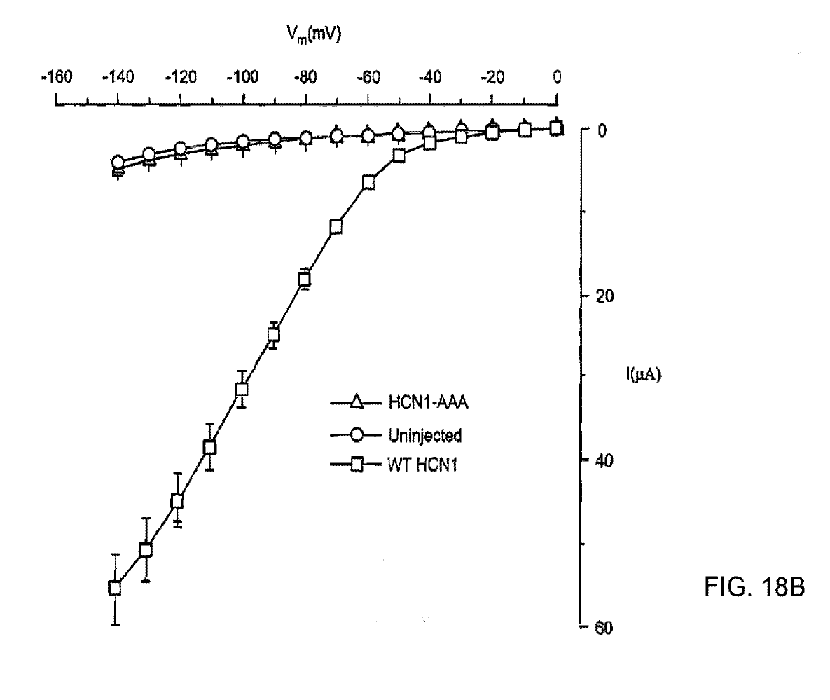

FIG. 23 shows the effects of E235 mutations on HCN1 activation gating. A) Representative records of currents through E235A and E235R HCN1 channels elicited using the voltage protocol in FIG. 18. B) Steady-state activation curve of WT and E235A. The activation curve for E235A is shifted positively. C) Steady-state activation curve of WT and E235R. The activation curve for E235R is shifted even more positively than that of E235A, showing a greater effect with a net charge change of +2 as compared to +1. D) Steady-state activation curves of WT, S253A, S253K and S253E channels. The conservative S-to-A mutant shows a shift of activation but has a preserved slope factor and P_{o,min}. Despite the opposite charges of these substitutions, the activation curves for both S253K and S253E are shifted far negatively. Taken collectively, this shows that the activation threshold of HCN channel activity (FIG. 23) can be modulated as well as the endogenous expressed current amplitude (FIGS. 18-22).

### DETAILED DESCRIPTION OF THE PREFERRED EMBODIMENT

### Cardiac Arrhythmia Treatment Methods

As discussed, several embodiments of the invention provide methods for the prevention or treatment of cardiac arrhythmia in a subject mammal. The term “treat” (or treatment) shall be given its ordinary meaning and shall include to reduce the severity of, prolong onset, or eliminate a symptom or disease (such as one or a combination of cardiac arrhythmias). Preferred methods involve administering a therapeutically effective amount of at least one polynucleotide capable of modulating at least one heart electrical property. More preferred methods involve expression of the polynucleotide sufficient to prevent or treat the cardiac arrhythmia in the mammal.

Preferred mammals include domesticated animals e.g., pigs, horses, dogs, cats, sheep, goats and the like; rodents such as rats, hamsters and mice; rabbits; and primates such as monkeys, chimpanzees etc. A highly preferred mammal is a human patient, preferably a patient who has or is suspected of having a cardiac arrhythmia. Methods of diagnosing and treating a variety of cardiac arrhythmias have been disclosed. See Cardiovascular Arrhythmias (1973) (Dreifus, L. S. and Likoff, W. eds) Grune & Stratton, NY, herein incorporated by reference; and references cited therein.

Several embodiments of the invention are generally compatible with one or a combination of suitable polynucleotide administration routes including those intended for in vivo or ex vivo cardiac use. As discussed, there is understanding in the field that cardiac tissue is especially amenable to gene transfer techniques. See e.g., Donahue, J. et al. (1998) Gene Therapy 5: 630; Donahue, J. et al. PNAS (USA) 94: 4664 (disclosing rapid and efficient gene transfer to the heart); Akhter, S. et al. (1997) PNAS (USA) 94: 12100 (showing successful gene transfer to cardiac ventricular myocytes), all herein incorporated by reference, and references cited therein.

See also the Examples and Drawings provided herein which demonstrate, inter alia, successful use of myocardial gene transfer techniques particularly to address cardiac arrhythmia.

Several embodiments of the invention feature administration routes in which expression of the introduced polynucleotide directly or indirectly causes a decrease in speed of conduction through at least one of: 1) the atrioventricular (AV) node (A-H interval) and 2) the His-Purkinje system. The decrease is readily detected and measured according to conventional means e.g., by use of one or more of the standard electrophysiological assays disclosed herein. Decreases of at least about 10% relative to baseline in the assay, preferably about 20% to 50% or more, are useful for many embodiments.

As will be appreciated, baseline values will often vary with respect to the particular polynucleotide(s) chosen. Methods to quantify baseline expression or protein include western blot, quantitative PCR, or functional assays such as adenylate cyclase assay for inhibitory G proteins, patch clamp analysis for ion channel currents. EP effects can be determined by measuring heart rate, conduction velocity or refractory period in vivo with EP catheters.

Additionally preferred methods include administration routes in which expression of the introduced polynucleotide directly or indirectly results in an increase in the AV node refractory period (AVNERP) as measured by the assay. An increase of at least about 10% relative to baseline in the assay, preferably at least about 20% to about 50% or more, will be preferred in many invention embodiments. Conventional methods for detecting and measuring the AVNERP are known and include the standard electrophysiological tests referenced herein.

Further preferred administration routes according to several embodiments of the invention involve introducing the polynucleotide into cardiac tissue and expressing same sufficient to detectably decrease heart rate as determined by a standard electrocardiogram (ECG) recording. Preferably, the decrease in heart rate is at least about 5% relative to baseline. Also preferably, the decrease in ventricular response rate or pulse during the cardiac arrhythmia (e.g., atrial fibrillation) is at least about 10% relative to baseline as determined by the recording.

As will be apparent, several embodiments of the invention are highly flexible and can be used with one or a combination of polynucleotides, preferably those encoding at least one therapeutic heart protein. A more preferred polynucleotide: 1) either decreases the A-H interval or increases the AVNERP by at least about 10%, preferably at least about 20% to about 50%, as determined by the electrophysiological assay; and 2) decreases ventricular response rate or pulse rate during atrial fibrillation by at least about 10%, preferably at least about 20% to about 50% as determined by a standard electrocardiogram (ECG) recording.

Additionally preferred polynucleotides include, but are not limited to, those encoding at least one ion channel protein, gap junction protein, G protein subunit, connexin; or functional fragment thereof. More preferred are polynucleotides encoding a K channel subunit, Na channel subunit, Ca channel subunit, an inhibitory G protein subunit; or a functional fragment thereof. Additionally preferred polynucleotides will encode one, two or three of such proteins (the same or different). However polynucleotides encoding one of those proteins will be preferred for most invention applications.

By the phrase “function fragment” is meant a portion of an amino acid sequence (or polynucleotide encoding that sequence) that has at least about 80%, preferably at least about 95% of the function of the corresponding fall-length amino acid sequence (or polynucleotide encoding that sequence). Methods of detecting and quantifying functionality in such fragments are known and include the standard electrophysiological assays disclosed herein.

For example, in embodiments in one or more of the polynucleotides encodes an inhibitory G protein, a suitable test for assaying function of that protein (as well as functional fragments thereof) is the adenylate cyclase assay disclosed by Sugiyama A. et al. in J Cardiovasc Pharm 1997; 29:734, herein incorporated by reference.

Suitable polynucleotides for practicing several embodiments of the invention can be obtained from a variety of public sources including, but not limited to, GenBank (National Center for Biotechnology Information (NCBI)), EMBL data library, SWISS-PROT (University of Geneva, Switzerland), the PIR-International database; and the American Type Culture Collection (ATCC) (10801 University Boulevard, Manassas, Va. 20110-2209), herein incorporated by reference. See generally Benson, D. A. et al. (1997) Nucl. Acids. Res. 25: 1 for a description of Genbank, herein incorporated by reference.

More particular polynucleotides for use with embodiments of the present invention are readily obtained by accessing public information from GenBank. For example, in one approach, a desired polynucleotide sequence is obtained from GenBank. The polynucleotide itself can be made by one or a combination of routine cloning procedures including those employing PCR-based amplification and cloning techniques. For example, preparation of oligonucleotide sequence, PCR amplification of appropriate libraries, preparation of plasmid DNA, DNA cleavage with restriction enzymes, ligation of DNA, introduction of DNA into a suitable host cell, culturing the cell, and isolation and purification of the cloned polynucleotide are known techniques. See e.g., Sambrook et al. in Molecular Cloning: A Laboratory Manual (2d ed. 1989); and Ausubel et al. (1989), Current Protocols in Molecular Biology, John Wiley & Sons, New York, herein incorporated by reference.

Table 1 below, references illustrative polynucleotides from the GenBank database for use with embodiments of the present invention.

TABLE 1

| Poly nucleotide | GenBank Accession No. |
|---|---|
| Human Gi2 protein alpha subunit sequence: | AH001470 |
| Kir 2.1 potassium channel | XM028411¹ |
| HERG potassium channel | XM004743 |
| Connexin 40 | AF151979 |
| Connexin 43 | AF151980 |
| Connexin 45 | U03493 |
| Na channel alpha subunit | NM000335 |
| Na channel beta-1 subunit | NM001037 |
| L-type Ca channel alpha-1 subunit | AF201304 |

¹An additional polynucleotide for use with the present invention is the Kir2.1 AAA mutant, which is wild-type Kir 2.1 with a substitution mutation of AAA for GFG in position 144 146.

Additional polynucleotides for use with several embodiments of the invention have been reported in the following references: Wong et al. Nature 1991; 351(6321):63 (constitutively active Gi2 alpha);) De Jongh K S, et al. J Biol Chem 1990 Sep. 5; 265(25):14738 (Na and Ca channel beta subunits); Perez-Reyes, E. et al. J Biol Chem 1992 Jan. 25; 267(3):1792; Neuroscientist 2001 February; 7(1):42 (providing sodium channel beta subunit information); Isom, L L. Et al. Science 1992 May 8; 256(5058):839 providing the beta 1 subunit of a brain sodium channel); and Isom, L L. Et al. (19.95) Cell 1995 Nov. 3; 83(3):433 (reporting beta 2 subunit of brain sodium channels), all herein incorporated by reference.

Further polynucleotides for use with several embodiments of the invention have been reported in PCT application number PCT/US98/23877 to Marban, E, herein incorporated by reference.

See also the following references authored by E. Marban: J. Gen Physiol. 2001 August; 118(2):171 82; Circ Res. 2001 Jul. 20; 89(2):160 7; Circ Res. 2001 Jul. 20; 89(2):101; Circ Res. 2001 Jul. 6; 89(1):33 8; Circ Res. 2001 Jun. 22; 88(12):1267 75; J. Biol. Chem. 2001 Aug. 10; 276(32):30423 8; Circulation. 2001 May 22; 103(20):2447 52; Circulation. 2001 May 15; 103(19):2361 4; Am J Physiol Heart Circ Physiol. 2001 June; 280(6):H2623 30; Biochemistry. 2001 May 22; 40(20):6002 8; J. Physiol. 2001 May 15; 533(Pt 1):127 33; Proc Natl Acad Sci USA. 2001 Apr. 24; 98(9):5335 40; Circ Res. 2001 Mar. 30; 88(6):570 7; Am J Physiol Heart Circ Physiol. 2001 April; 280(4):H 1882 8; and J Mol Cell Cardiol. 2000 November; 32(11):1923 30, all herein incorporated by reference.

Further examples of suitable Ca channel subunits include beta 1, or alpha2-delta subunit from an L-type Ca channel. A preferred Na channel subunit is beta1 or beta2. In some invention embodiments it will be useful to select Na and Ca channel subunits having dominant negative activity as determined by the standard electrophysiological assay described below. Preferably, that activity suppresses at least about 10% of the activity of the corresponding normal Na or Ca channel subunit as determined in the assay.

Also preferred is the inhibitory G protein subunit (“Gαᵢ₂”) or a functional fragment thereof.

Several embodiments of the invention are broadly suited for use with gap junction proteins, especially those known or suspected to be involved with cardiac function. Particular examples include connexin 40, 43, 45; as well as functional fragments thereof. Further contemplated are polynucleotides that encode a connexin having dominant negative activity as determined by the assay, preferably a suppression activity of at least about 10% with respect to the corresponding normal connexin 40, 43, or 45.

Also envisioned are mutations of such polynucleotides that encode dominant negative proteins (muteins) that have detectable suppressor activity. Encoded proteins that are genetically dominant typically inhibit function of other proteins particularly those proteins capable of forming binding complexes with the wild-type protein.

Additional polynucleotides of the invention encode essentially but not entirely full-length protein. That is, the protein may not have all the components of a full-length sequence. For example, the encoded protein may include a complete or nearly complete coding sequence (cds) but lack a complete signal or poly-adenylation sequence. It is preferred that a polynucleotide and particularly a cDNA encoding a protein of several embodiments of the invention include at least a complete cds. That cds is preferably capable of encoding a protein exhibiting a molecular weight of between about 0.5 to 70, preferably between about 5 and 60, and more preferably about 15, 20, 25, 30, 35, 40 or 50 kD. That molecular weight can be readily determined by suitable computer-assisted programs or by SDS-PAGE gel electrophoresis.

Although generally not preferred, the nucleic acid segment can be a genomic sequence or fragment thereof comprising one or more exon sequences. In this instance it is preferred that the cell, tissue or organ selected for performing somatic cell gene transfer be capable of correctly splicing any exon sequences so that a full-length or modified protein can be expressed.

The polynucleotide and particularly the cDNA encoding the full-length protein can be modified by conventional recombinant approaches to modulate expression of that protein in the selected cells, tissues or organs.

More specifically, suitable polynucleotides can be modified by recombinant methods that can add, substitute or delete one or more contiguous or non-contiguous amino acids from that encoded protein. In general, the type of modification conducted will relate to the result of expression desired.

For example, a cDNA polynucleotide encoding a protein of interest such as an ion channel can be modified so as overexpress that protein relative to expression of the full-length protein (e.g., control assay). Typically, the modified protein will exhibit at least 10 percent or greater overexpression relative to the full-length protein; more preferably at least 20 percent or greater; and still more preferably at least about 30, 40, 50, 60, 70, 80, 100, 150, or 200 percent or greater overexpression relative to the control assay.

As noted above, further contemplated modifications to a polynucleotide (nucleic acid segment) and particularly a cDNA are those which create dominant negative proteins.

In general, a variety of dominant negative proteins can be made by methods known in the field. For example, ion channel proteins are recognized as one protein family for which dominant negative proteins can be readily made, e.g., by removing selected transmembrane domains. In most cases, the function of the ion channel binding complex is substantially reduced or eliminated by interaction of a dominant negative ion channel protein.

Several specific strategies have been developed to make dominant negative proteins. Exemplary of such strategies include oligonucleotide directed and targeted deletion of cDNA sequence encoding the desired protein. Less preferred methods include nucleolytic digestion or chemical mutagenesis of the cDNA.

It is stressed that creation of a dominant negative protein is not synonymous with other conventional methods of gene manipulation such as gene deletion and antisense RNA. What is meant by “dominant negative” is specifically what is sometimes referred to as a “poison pill” which can be driven (e.g., expressed) by an appropriate DNA construct to produce a dominant negative protein which has capacity to inactivate an endogenous protein.

For example, in one approach, a cDNA encoding a protein comprising one or more transmembrane domains is modified so that at least 1 and preferably 2, 3, 4, 5, 6 or more of the transmembrane domains are eliminated. Preferably, the resulting modified protein forms a binding complex with at least one other protein and usually more than one other protein. As noted, the modified protein will inhibit normal function of the binding complex as assayed, e.g., by standard ligand binding assays or electrophysiological assays as described herein. Exemplary binding complexes are those which participate in electrical charge propagation such as those occurring in ion channel protein complexes. Typically, a dominant negative protein will exhibit at least 10 percent or greater inhibition of the activity of the binding complex; more preferably at least 20 percent or greater; and still more preferably at least about 30, 40, 50, 60, 70, 80, or 100 percent or greater inhibition of the binding complex activity relative to the full-length protein.

As a further illustration, a cDNA encoding a desired protein for use in the present methods can be modified so that at least one amino acid of the protein is deleted. The deleted amino acid(s) can be contiguous or non-contiguous deletions essentially up to about 1%, more preferably about 5%, and even more preferably about 10, 20, 30, 40, 50, 60, 70, 80, or 95% of the length of the full-length protein sequence.

Alternatively, the cDNA encoding the desired protein can be modified so that at least one amino acid in the encoded protein is substituted by a conservative or non-conservative amino acid. For example, a tyrosine amino acid substituted with a phenylalanine would be an example of a conservative amino acid substitution, whereas an arginine replaced with an alanine would represent a non-conservative amino acid substitution. The substituted amino acids can be contiguous or non-contiguous substitutions essentially up to about 1%, more preferably about 5%, and even more preferably about 10, 20, 30, 40, 50, 60, 70, 80, or 95% of the length of the full-length protein sequence.

Although generally less-preferred, the nucleic acid segment encoding the desired protein can be modified so that at least one amino acid is added to the encoded protein. Preferably, an amino acid addition does not change the ORF of the cds. Typically, about 1 to 50 amino acids will be added to the encoded protein, preferably about 1 to 25 amino acids, and more preferably about 2 to 10 amino acids. Particularly preferred addition sites are at the C- or N-terminus of the selected protein.

Preferred invention practice involves administering at least one of the foregoing polynucleotides with a suitable a myocardium nucleic acid delivery system. In one embodiment, that system includes a non-viral vector operably linked to the polynucleotide. Examples of such non-viral vectors include the polynucleoside alone or in combination with a suitable protein, polysaccharide or lipid formulation.

Additionally suitable myocardium nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomeglovirus (CMV) promoter.

Additionally preferred vectors include viral vectors, fusion proteins and chemical conjugates. Retroviral vectors include moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci. USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69:2004 (1995)] and Adeno-associated Virus Vectors [Kaplitt, M. G., et al., Nat. Genet. 8:148 (1994)], all herein incorporated by reference.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are may be indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. Preferred in vivo or ex vivo cardiac administration techniques have already been described.

To simplify the manipulation and handling of the polynucleotides described herein, the nucleic acid is preferably inserted into a cassette where it is operably linked to a promoter. The promoter must be capable of driving expression of the protein in cells of the desired target tissue. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)), herein incorporated by reference, and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618, herein incorporated by reference.

If desired, the polynucleotides of several embodiments of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989), all herein incorporated by reference.

Replication-defective recombinant adenoviral vectors, can be produced in accordance with known techniques. See, Quantin, et al., Proc. Natl. Acad. Sci. USA, 89:2581 2584 (1992); Stratford-Perricadet, et al., J. Clin. Invest., 90:626 630 (1992); and Rosenfeld, et al., Cell, 68:143 155 (1992), all herein incorporated by reference.

The effective dose of the nucleic acid will be a function of the particular expressed protein, the particular cardiac arrhythmia to be targeted, the patient and his or her clinical condition, weight, age, sex, etc.

One preferred myocardicum delivery system is a recombinant viral vector that incorporates one or more of the polynucleotides therein, preferably about one polynucleotide. Preferably, the viral vector used in several embodiments of the invention has a pfu (plague forming units) of from about 10⁸ to about 5×10¹⁰ pfu. In embodiments, in which the polynucleotide is to be administered with a non-viral vector, use of between from about 0.1 nanograms to about 4000 micrograms will often be useful e.g., about 1 nanogram to about 100 micrograms.

Choice of a particular myocardium delivery system will be guided by recognized parameters including the cardiac arrhythmia of interest and the amount and length of expression desired. Use of virus vectors approved for human applications e.g., adenovirus are particularly preferred.

As discussed, it is an object of several embodiments of the invention to prevent or treat cardiac arrhythmia. In one embodiment, the method further includes overexpressing a potassium (K) channel protein subunit sufficient to decrease cardiac action potential duration (APD) by at least about 5% as determined by the standard cardiac electrophysiological assay.

Reference herein to an electrophysiological assay is meant a conventional test for determining cardiac action potential (AP). See generally Fogoros R N. Electrophysiologic Testing Blackwell Science, Inc. (1999) for disclosure relating to performing such tests.

Specific reference herein to a “standard electrophysiological assay” is meant the following general assay.

1) providing a mammalian heart (in vivo or ex vivo),

2) contacting the heart with at least one suitable polynucleotide preferably in combination with an appropriate myocardium nucleic acid delivery system,

3) transferring the polynucleotide into cells of the heart under conditions which allow expression of the encoded amino acid sequence; and

4) detecting modulation (increase or decrease) of at least one electrical property in the transformed heart e.g., at least one of conduction, ventricular response rate, and pulse rate.

Particular embodiments include modifying the polynucleotide along lines discussed above sufficient to overexpress the encoded protein. Further preferred are methods in which the nucleic acid is modified to produce a dominant negative ion channel protein. The ion channel protein can be e.g., a sodium, calcium, voltage-gated, or ligand-gated ion channel and particularly a potassium ion channel. Additional disclosure relating to such channel proteins can be found in the discussion above and in U.S. Pat. No. 5,436,128, for instance.

Practice of several embodiments of the invention is broadly compatible with one or a combination of different administration (delivery) systems.

In particular, one suitable administration route involves one or more appropriate polynucleotide into myocardium. Alternatively, on in addition, the administration step includes perfusing the polynucleotide into cardiac vasculature. If desired, the administration step can further include increasing microvascular permeability using routine procedures, typically administering at least one vascular permeability agent prior to or during administration of the gene transfer vector. Examples of particular vascular permeability agents include administration of one or more of the following agents preferably in combination with a solution having less than about 500 micromolar calcium: substance P, histamine, acetylcholine, an adenosine nucleotide, arachidonic acid, bradykinin, endothelin, endotoxin, interleukin-2, nitroglycerin, nitric oxide, nitroprusside, a leukotriene, an oxygen radical, phospholipase, platelet activating factor, protamine, serotonin, tumor necrosis factor, vascular endothelial growth factor, a venom, a vasoactive amine, or a nitric oxide synthase inhibitor. A particular is serotonin, vascular endothelial growth factor (VEGF), or a functional VEGF fragment to increase the permeability.

Typical perfusion protocols in accord with several embodiments of the invention are generally sufficient to transfer the polynucleotide to at least about 10% of cardiac myocytes in the mammal. Infusion volumes of between from about 0.5 to about 500 ml are preferred. Also preferred are coronary flow rates of between from about 0.5 to about 500 ml/min. Additionally preferred perfusion protocols involve the AV nodal artery. Transformed heart cells, typically cardiac myocytes that include the polynucleotide are suitably positioned at or near the AV node.

Illustrative strategies for detecting modulation of transformed heart have been disclosed e.g., in Fogoros R N, supra. A preferred detection strategy is performing a conventional electrocardiogram (ECG). Modulation of cardiac electrical properties by use of several embodiments of the invention is readily observed by inspection of the ECG. See also the Examples and Drawings below.

More specific methods for preventing or treating cardiac arrhythmia include overexpressing a K channel protein subunit sufficient to decrease surface electrocardiogram (ECG) repolarization time by at least about 5%, preferably at least about 10% to about 20%, as determined by the assay. Typically, the K channel protein subunit is overexpressed by at least about 2 fold, preferably about 5 fold, relative to an endogenous K channel protein as determined by a standard Northern or Western blot assay. Also preferably, the K channel protein subunit is overexpressed and impacts repolarization in congestive heart failure or myocardial infarction in the long QT syndrome.

In particular embodiments, methods for preventing or treating cardiac arrhythmia provided herein further include decreasing conduction through cardiac tissues by at least about 5%, preferably at least about 10% to about 20%, as determined by the standard electrophysiological assay.

As discussed, several embodiments of the invention is one of general application that can be used to treat one or a combination of different cardiac arrhythmias. Examples of particular arrhythmias has been disclosed by Bigger, J. T and B. F. Hoffman, supra. More specific examples include atrial flutter, atrial fibrillation, and ventricular tachycardia. Other examples include sinus bradycardia, sinus tachycardia, atrial tachycardia, atrial fibrillation, atrial flutter, atrioventricular nodal block, atrioventricular node reentry tachycardia, atrioventricular reciprocating tachycardia, ventricular tachycardia or ventricular fibrillation.

The following sections 1-5 discuss particular uses of embodiments of the present invention.

1. Sinus Bradycardia: Direct injection or intravascular perfusion of materials/vectors into the atria or ventricles in order to create a discrete focus of electrically active tissue to replace the function of the sinus node. Indications might include: sick sinus syndrome, Stokes-Adams attacks, syncope, chronic fatigue syndrome, cardiomyopathies (hypertrophic and dilated), and all other present and future indications for electronic pacemakers. Therapeutic genes could include wild-type or mutated potassium, HCN and/or calcium channel subunits to increase local automaticity and/or to induce pacemaker activity where it is not normally present.

2. Inappropriate Sinus Tachycardia: Modification of the automaticity in the sinus node and/or surrounding atrial tissue for the treatment of inappropriate sinus tachycardia, e.g. by introducing K channel, Ca channel or HCN channel genes to decrease nodal excitability.

3. Atrial Fibrillation/Atrial Flutter/Atrial Tachycardia: Direct injection or intravascular perfusion of materials/vectors in order to: (1) produce lines of conduction block in order to prevent conduction of reentry-type atrial arrhythmias, (2) suppress automaticity or increase refractoriness in order to ablate discrete arrhythmic foci of tissue, (3) affect conduction velocity, refractoriness or automaticity diffusely throughout the atria in order to prevent or treat atrial fibrillation, multifocal atrial tachycardia or other atrial tachycardias with multiple or diffuse mechanisms, or (4) Direct injection into the atrioventricular node or perfusion of the atrioventricular nodal artery with materials/vectors to alter the conduction properties (conduction velocity, automaticity, refractoriness) of the atrioventricular node in order to slow the ventricular response rate to atrial arrhythmias.

4. Atrioventricular nodal block: Direct injection or intracoronary perfusion of materials/vectors into the atrioventricular nodal region or into the ventricles in order to (1) create a discrete focus of electrically active tissue to initiate the heart beat in the absence of atrioventricular nodal conduction of the normal impulse from the atria, or (2) reestablish function of the atrioventricular node.

5. Ventricular Tachycardia/Ventricular Fibrillation: Delivery of vectors by: (1) Direct injection into discrete foci of ventricular myocardium to suppress automaticity or increase refractoriness in order to ablate arrhythmic foci by genetic means, (2) Diffuse direct injection or coronary artery perfusion of materials/vectors into both ventricles to affect the conduction properties (conduction velocity, automaticity, refractoriness) of ventricular tissue in order to treat or prevent ventricular arrhythmias, or (3) Direct injection of materials/vectors to produce lines of conduction block in order to prevent conduction of reentry-type ventricular arrhythmias.

As also discussed, several embodiments of the present invention provides more specific methods for preventing or treating ventricular rate or pulse during atrial fibrillation. In one embodiment, the method includes administering to the mammal a therapeutically effective amount of at least one polynucleotide encoding a Gαᵢ₂ subunit or a functional fragment thereof. Typically preferred methods further include expressing the polynucleotide in the mammal to prevent or treat the atrial fibrillation. Preferred methods also include overexpressing the Gαᵢ₂ subunit or a functional fragment thereof sufficient to decrease speed of conduction through the atrioventricular (AV) node (A-H interval) or His-Purkinje system as determined by a standard electrophysiological assay. Also preferably, the decrease in the A-H interval is accompanied by an increase in AV node refractory period (AVNERP). The decrease in the A-H interval is at least about 10%, preferably at least about 20%, as determined by the assay. The increase in AVNERP is at least about 10%, preferably at least about 20%, as determined by the assay.

By the phrase “therapeutically effective” amount or related phrase is an amount of administered polynucleotide needed to achieve a desired clinical outcome.

In one embodiment of the foregoing specific method, overexpression of the Gαᵢ₂ or a functional fragment thereof is capable of decreasing pulse rate or ventricular rate during atrial fibrillation as determined by a standard cardiac electrophysiological assay. Preferably, the decrease in pulse rate or ventricular rate during atrial fibrillation is at least about 10%, preferably at least about 20%, as determined by the assay.

The foregoing embodiments of the invention for preventing or treating atrial fibrillation provide specific advantages. For example, it has been found that it is possible to transfer genes to half of AV nodal cells with clinically relevant delivery parameters. Desirable therapeutic effects of the gene therapy include slowing of AV nodal conduction and increases of the refractory period of the AV node, with resultant slowing of the ventricular response rate during atrial fibrillation. The work provides proof of principle that gene therapy is a viable option for the treatment of common arrhythmias.

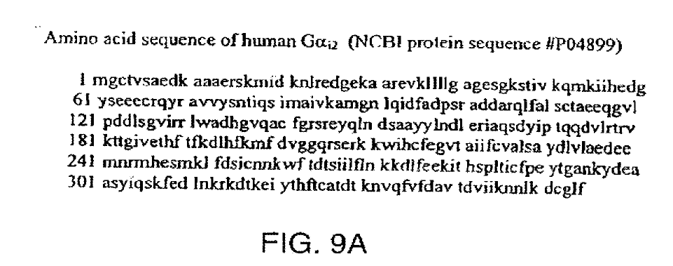

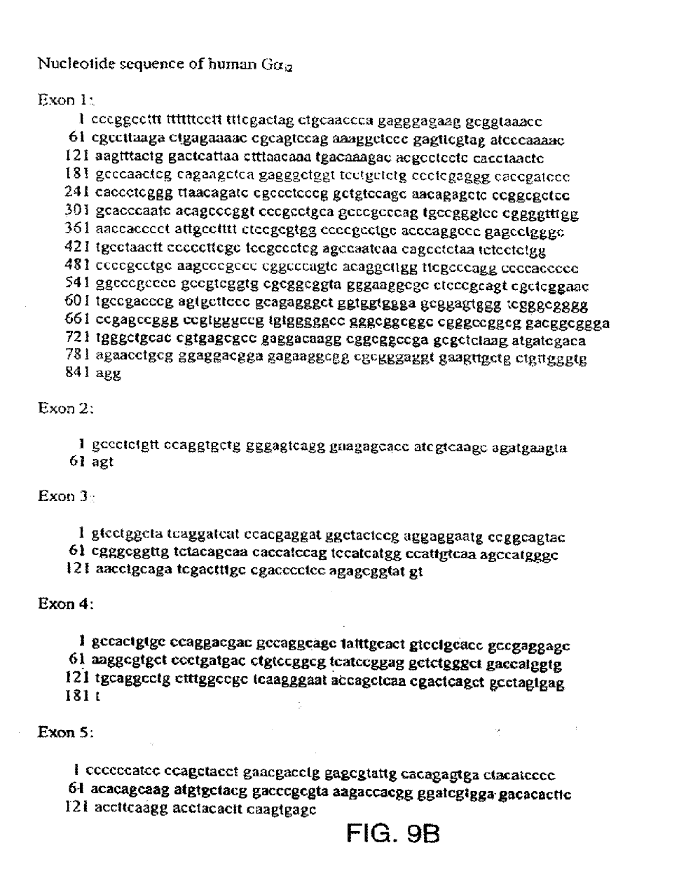

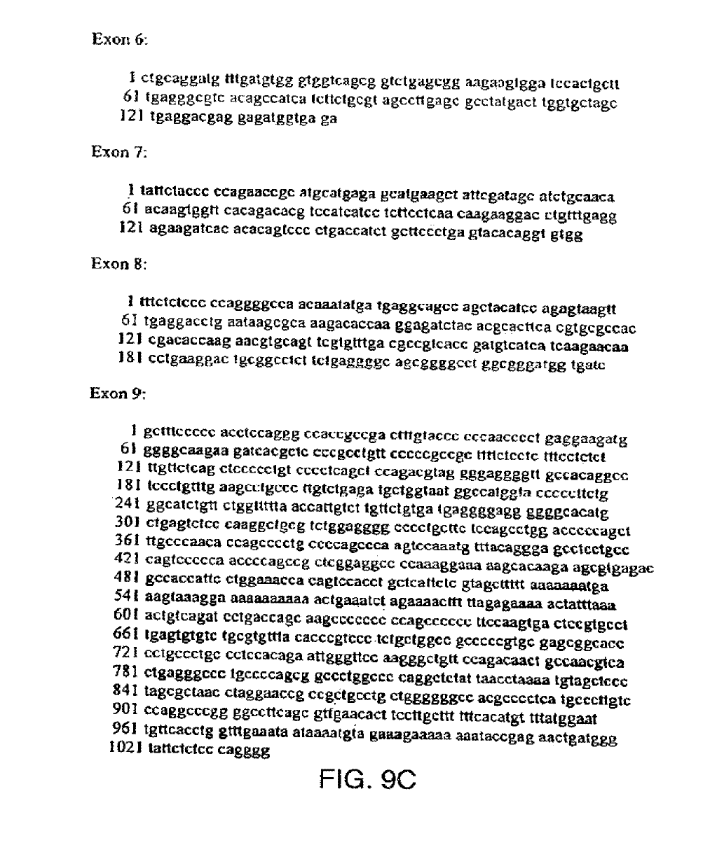

In one invention embodiment, the polynucleotide encoding the Gαᵢ₂ subunit hybridizes to the nucleic acid sequence shown in FIGS. 9B-C (SEQ ID NO's: 1-9, respectively in order of appearance); or the complement thereof under high stringency hybridization conditions. Encoded amino acid sequence is shown in FIG. 9A (SEQ ID NO. 10). By the phrase “high stringency” hybridization conditions is meant nucleic acid incubation conditions approximately 65.degree. C. in 0.1×SSC. See Sambrook, et al., infra. Preferably, the polynucleotide consists of or comprises the nucleic acid shown in FIGS. 9B-C (SEQ ID NO's: 1-9, respectively in order of appearance). FIGS. 9A-C show the subunit nucleotide sequence as exon representations. It will be appreciated that in the gene sequence, the exons are covalently linked together end-to-end (exon 1, 2,etc).

As discussed, it is an object of one embodiment of the present invention to use gene therapy as an antiarrhythmic strategy. The Examples section, in particular, focuses genetic modification of the AV node. An intracoronary perfusion model for gene delivery, building on previous work in isolated cardiac myocytes and ex vivo-perfused hearts has been developed^{4,5}. Using this method, porcine hearts were infected with Adβgal (a recombinant adenovirus expressing E. coli β-galactosidase) or with AdGᵢ (encoding the Gαᵢ₂ subunit). Gαᵢ₂ overexpression suppressed baseline AV conduction and slowed the heart rate during atrial fibrillation, without producing complete heart block. In contrast, expression of the reporter gene β-galactosidase had no electrophysiological effects. These results demonstrate the feasibility of using myocardial gene transfer strategies to treat common arrhythmias.

More generally, several embodiments of the invention can be used to deliver and express a desired ion channel, extracellular receptor, or intracellular signaling protein gene in selected cardiac tissues, particularly to modify the electrical properties of that tissue, e.g., increasing or decreasing its refractoriness, increasing or decreasing the speed of conduction, increasing or decreasing focal automaticity, and/or altering the spatial pattern of excitation. The general method involves delivery of genetic materials (DNA, RNA) by injection of the myocardium or perfusion through the vasculature (arteries, veins) or delivery by nearly any other material sufficient to facilitate transformation into the targeted portion of the myocardium using viral (adenovirus, AAV, retrovirus, HVJ, other recombinant viruses) or non-viral vectors (plasmid, liposomes, protein-DNA combinations, lipid-DNA or lipid-virus combinations, other non-viral vectors) to treat cardiac arrhythmias.

By way of illustration, genes that could be used to affect arrhythmias include ion channels and pumps (α subunits or accessory subunits of the following: potassium channels, sodium channels, calcium channels, chloride channels, stretch-activated cation channels, HCN channels, sodium-calcium exchanger, sodium-hydrogen exchanger, sodium-potassium ATPase, sarcoplasmic reticular calcium ATPase), cellular receptors and intracellular signaling pathways (α or β-adrenergic receptors, cholinergic receptors, adenosine receptors, inhibitory G protein α subunits, stimulatory G protein α subunits, Gβγ subunits) or genes for proteins that affect the expression, processing or function processing of these proteins.

Selection of the appropriate gene(s) for therapy can be performed by anyone with an elementary knowledge of cardiac electrophysiology. In addition, the effects of ion channel expression can be simulated by computer programs to anticipate the effects of gene transfer. The delivery methods for myocardial delivery are widely reported, and methods involving injection of the myocardium or intravascular perfusion have been used successfully.

More specific advantages of several embodiments of the invention include ability to convey localized effects (by focal targeted gene delivery), reversible effects (by use of inducible vectors, including those already reported as well as new generations of such vectors, including but not limited to adeno-associated vectors using tetracycline-inducible promoters to express wild-type or mutant ion channel genes), gradedness (by use of inducible vectors as noted above, in which gradedness would be achieved by titration of the dosage of the inducing agent), specificity of therapy based on the identity of the gene construct, ability to regulate therapeutic action by endogenous mechanisms (nerves or hormones) based on the identity of the gene construct, and avoidance of implantable hardware including electronic pacemakers and AICDs, along with the associated expense and morbidity.

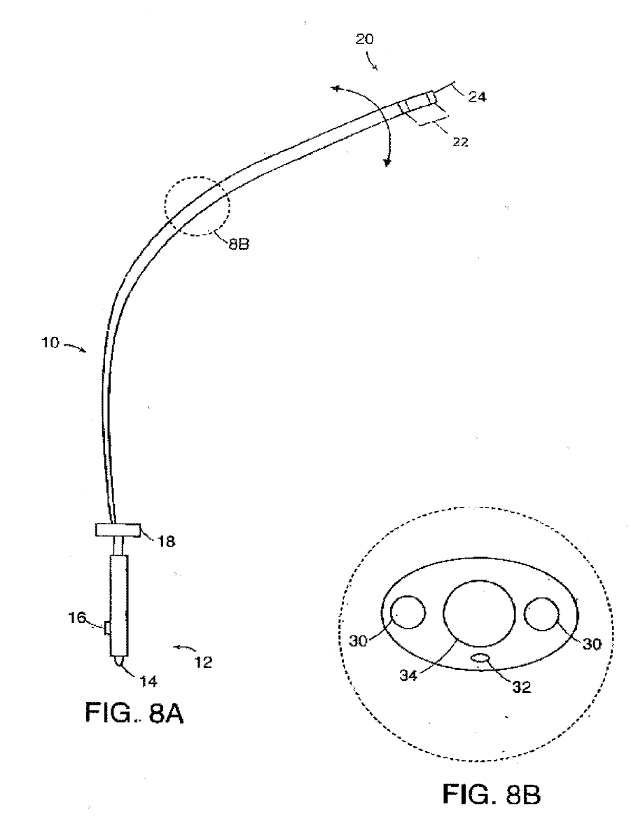

As discussed above, several embodiments of the invention also include devices useful in the treatment methods of several embodiments of the invention. These devices include catheters that include in a single unitary unit that contain both delivery and position detection features. FIGS. 8A and 8B show catheter unit 10 that contains at proximal end 12 (e.g., end manipulated by medical practitioner, typically external to patient) electrical connection 14, therapeutic agent injection port and needle extension mechanism 16, and steering control 18. Distal end 20 of catheter 10 includes electrodes 22 for detection of the distal end position within a patient and retractable needle 24 for delivery of the therapeutic agent, particularly a polynucleotide to targeted tissue, especially a polynucleotide to mammalian cardiac tissue. The needle 24 can be manipulated by extension mechanism 16. Connection 14 enables activation of detection apparatus 22. A therapeutic agent such as a polynucleotide can be injected or otherwise introduced into device 10 via injection port 16. FIG. 8B shows the specified catheter region in cross-section with electrode cables 30 that provide communication between electrical connection 14 and electrodes 22, steering rod 32 that can enable manipulation of catheter 10 within the patient via steering control 14, and injector connection or tubing 34 that provides a path for delivery of the therapeutic agent through catheter 10 to the targeted tissue of the patient. The device is suitably employed in a minimally invasive (endoscopic) procedure.

Variations of the depicted design also will be suitable. For instance, the catheter may comprise a tip (distal portion) with a fixed curve. Additionally, rather than having the therapeutic agent traverse the catheter 10, the agent may be housed within a reservoir, which may be activated (e.g., therapeutic agent released to patient) via mechanism at catheter proximal end. The needle 24 may be a straight needle or a screw-type apparatus. In each design, the device suitable contains some type of detection apparatus, e.g. electrodes that provide for electrophyiologically-guided substance injections into the targeted tissue. The following specific examples are illustrative of several embodiments of the invention.

### Example 1

### Gene Transfer of β-Galactosidase (β-Gal) and Inhibitory G Protein Subunit (Gαᵢ₂) into Cardiac Tissue

In previous ex vivo and in vitro studies, we found that gene transfer efficiency correlated with coronary flow rate, virus exposure time, virus concentration, and the level of microvascular permeability^{4,5}. We also found that elimination of radiographic contrast media and red blood cells from the perfusate and delivery at body temperature were necessary for optimal results. The in vivo delivery system used in this report builds on those findings.

Ten animals underwent a protocol that included medication with oral sildenafil before baseline electrophysiology (EP) study, catheterization of the right coronary artery, and infusion of VEGF, nitroglycerin and virus-containing solutions (7.5×10⁹ pfu in 1 ml) into the AV nodal branch of the right coronary artery. VEGF was used to increase microvascular permeability⁶, and sildenafil potentiated the VEGF effect. The infusion volume and coronary flow rate were limited to avoid efflux from the artery and infection of other regions of the heart. Five animals received Adβgal, and the other 5 received AdGᵢ. The animals underwent follow-up EP study 7 days after virus infusion. After the second EP study, the hearts were explanted and evaluated for β-galactosidase (β-gal) and Gαᵢ₂ expression. Other adenoviral gene transfer studies have shown that expression is detectable after 3 days, peaks after 5 7 days, and then regresses over 20 30 days⁷⁻⁹. Based on these data, we tested for gene expression and phenotypic changes 7 days after gene delivery.

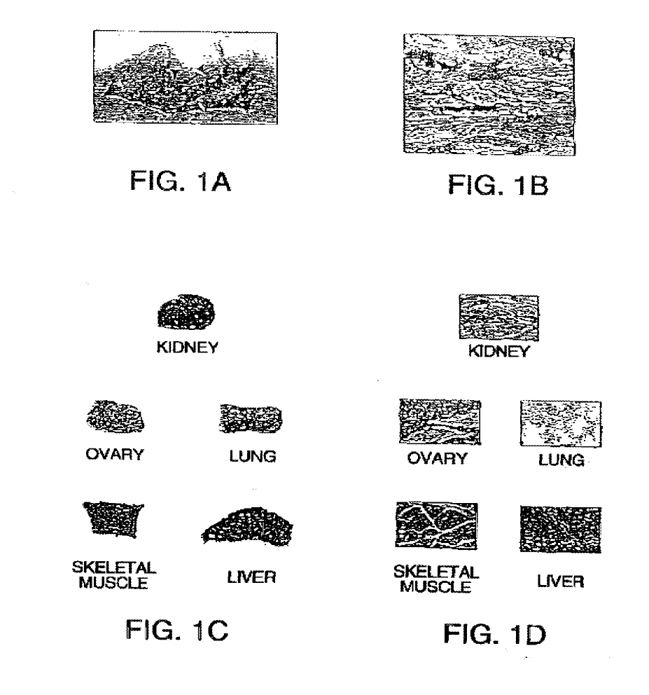

X-gal staining revealed β-gal activity in the AV nodal region and adjacent ventricular septum of all Adβgal-infected animals (FIG. 1a). There was no evidence of β-gal activity in any of the AdGᵢ-infected animals or in other heart sections from the Adβgal group. Microscopic sections through the AV node documented gene transfer to 45.+−0.6% of the AV nodal cells in the Adβgal group and confirmed the absence of X-gal staining in the AdGᵢ-infected animals. Also notable in the microscopic sections was a mild inflammatory infiltrate, comprised mainly of mononuclear cells.

Western blot analysis was performed on tissue homogenates from the AV nodal region of 4 animals from each group (FIG. 1b). Densitometry analysis confirmed Gαᵢ₂ overexpression in the AdGᵢ group, amounting to a 5-fold increase in Gαᵢ₂ relative to the Adβgal animals (p=0.01). The level of Gαᵢ₂ in the Adβgal group was not different from that found in 2 uninfected control animals.

X-gal staining of gross and microscopic sections from the lung, liver, kidney, skeletal muscle and ovaries of all animals was performed to evaluate the extent of gene transfer outside the heart (FIG. 1c). In the Adβgal-infected animals, β-gal activity was evident in gross specimens from the liver, kidneys and ovaries, but not in the lungs or skeletal muscle. Microscopic sections revealed definite β-gal activity, but in less than 1% of the cells in these organs. X-gal staining was not found in any tissues of the AdGᵢ-infected or uninfected control animals. The lack of X-gal staining in AdGᵢ-infected and uninfected controls indicates that the results were specific for transgene expression and not from endogenous β-gal activity or false-positive staining. These results are consistent with a previous study documenting gene expression in peripheral organs after intracardiac injection of adenovirus¹⁰, and suggest that ongoing clinical gene therapy trials should consider the risks of non-target organ gene transfer.

FIGS. 1A-D are explained in more detail as follows. Measurement of gene transfer efficacy. FIG. 1A. X-gal staining of a transverse section through the AV groove. Arrowheads indicate the tricuspid valve ring, and the solid arrow marks the central fibrous body. The hollow arrow points to the AV node. FIG. 1B. A microscopic section through the AV node shows gene transfer to 45.+−0.6% of myocytes. Cells expressing β-galactosidase are stained blue. FIG. 1C. Gross and microscopic pathology after exposure of liver, kidney and ovary to X-gal solution. FIG. 1D. Microscopic sections show rare blue cells in these organs (arrowheads). Lung and skeletal muscle failed to show any evidence of gene transfer.

### Example 2

### Electrophysiological Analysis of Cardiac Tissue Transduced with β-gal or Inhibitory G Protein (Gαᵢ₂) Subunit

Electrophysiological measurements obtained at baseline and 7 days after infection are displayed in Table 2, below.

TABLE 2
Electrophysiological Parameters Before
and 7 Days After Gene Transfer Adβgal

| | Day | | | |
|---|---|---|---|---|
| | 0 | 7 | 0 | 7 |
| Heart rate during | 114 ± 5 | 111 ± 1 | 113 ±2 | 106 ± 4 |
| sinus rythm | | | | |
| ECG: P-R interval | 101 ± 1 | 99 ± 1 | 97 ± 2 | 109 ± 5* |
| QRS interval | 58 ± 2 | 54 ± 1 | 57 ± 1 | 56 ± 1 |
| Q-T interval | 296 ± 6 | 310 ± 2 | 288 ± 7 | 316 ± 6 |
| A-H interval | 61 ± 1 | 61 ± 1 | 60 ± 2 | 76 ± 3* |
| H-V interval | 25 ± 1 | 25 ± 1 | 26 ± 1 | 24 ± 1 |
| AVNERP | 226 ± 6 | 224 ± 4 | 226 ± 6 | 246 ± 3* |

mean ± s.e.m.,
n = 5 in each group,
*p ≦ 0.03;
AVNERP: AV node effective refractory period

ECG parameters were taken from the surface ECG, and the A-H and H-V intervals were recorded from an intracardiac catheter in the His-bundle position. (The A-H interval measures conduction time through the AV node, and the H-V interval is the conduction time through the His-Purkinje system.) The AV node effective refractory period (AVNERP) was measured by pacing the atria at a stable rate for 8 beats and then delivering premature atrial stimuli at progressively shorter intervals, noting the interval where the premature beat failed to conduct through the AV node. There were no significant differences in the electrophysiological parameters between groups at baseline. In the Adβgal group, comparison of baseline measurements to those taken 7 days after infection also failed to show any significant differences. In contrast, the follow-up study of the AdGᵢ group revealed significant prolongation in the P-R interval on the surface ECG (paired analysis, day 0: 97.+−0.2 msec, day 7: 109.+−0.4 msec, p=0.01), the A-H interval on the intracardiac electrogram (day 0: 60.+−0.2 msec, day 7: 76.+−0.3 msec, p=0.01) and the AVNERP (day 0: 226.+−0.6 msec, day 7: 246.+−0.3 msec, p=0.03), indicating both slowed conduction and increased refractoriness of the AV node after Gαᵢ₂ overexpression.

### Example 3

### Measurement of Heart Rate in Cardiac Tissue Transduced with β-gal or Inhibitory G Protein (Gαᵢ₂) Subunit

After measurement of basic electrophysiological intervals, we measured the heart rate during acute episodes of atrial fibrillation. Overexpression of Gαᵢ₂ in the AV node caused a 20% reduction in the ventricular rate during atrial fibrillation (day 0: 199.+−0.5 bpm, day 7: 158.+−0.2 bpm, p=0.005). This effect persisted in the setting of adrenergic stimulation. Administration of epinephrine (1 mg, IV) increased the atrial fibrillation heart rate in all animals, but the group overexpressing Gαᵢ₂, nevertheless, exhibited a 16% reduction in ventricular rate (day 0: 364.+−0.3 bpm, day 7: 308.+−0.2 bpm, p=0.005). In contrast, β-gal expression did not affect the heart rate during atrial fibrillation, either before (day 0: 194.+−0.8 bpm, day 7: 191.+−0.7 bpm, p=NS) or after epinephrine administration (day 0: 362.+−0.6 bpm, day 7: 353.+−0.5, p=NS).

To further evaluate the effect of Gαᵢ₂ overexpression on AV conduction, we analyzed the heart rate at various time points after induction of atrial fibrillation in the AdGᵢ-epinephrine group. These data indicate that the ventricular rate remains stable and that the beneficial suppression of heart rate from Gαᵢ₂ gene transfer is sustained through at least 3 minutes of observation. The episodes of atrial fibrillation often lasted longer than 3 minutes (see methods), but the period of observation was limited to ensure that the effects of epinephrine would be constant.

The choice of Gαᵢ₂ to suppress conduction was inspired by the success of β-blocking drugs at achieving that goal. In the AV node, β-adrenergic receptors are coupled to stimulatory G proteins (G_{S}). Stimulation of β-receptors activates G_{S}, releasing the Gα_{S}-subunit to stimulate adenylate cyclase¹¹. This process leads to a cascade of intracellular events causing an increase in conduction velocity and a shortening of the refractory period. β-blockers prevent the increase in AV nodal conduction by inhibiting receptor activation.

The intracellular processes responsive to G_{S} are counterbalanced by the activity of inhibitory G proteins (Gᵢ). In the AV node, Gᵢ are coupled to muscarinic M2 and adenosine A1 receptors¹¹. Gᵢ activation releases the Gαᵢ-subunit to bind and inhibit adenylate cyclase activity and the Gβγ-subunit to increase potassium conductance by direct action on acetylcholine-activated potassium channels. The cumulative effect of Gᵢ activation is a decrease in conduction through the AV node. In agreement with these known effects of the G protein cascade, our data show that overexpression of Gαᵢ₂ suppresses AV nodal conduction in the drug-free state and during adrenergic stimulation.

Under ordinary circumstances, Gαᵢ₂-mediated inhibition of adenylate cyclase requires receptor activation¹². In the current study, however, Gᵢ activity appears to be uncoupled from the receptor, since the inhibition occurs without exogenous M2 or A1 receptor stimulation. In the setting of 5-fold overexpression of Gαᵢ₂, normal cellular mechanisms may well be altered. Further study will be required to elucidate the changes in signal transduction that underlie the observed effects.

A principal focus of this study was to overcome the problem of vector delivery to the myocardium using minimally invasive techniques. By manipulation of the tissue and vascular dynamics, the β-galactosidase and Gαᵢ₂ genes were transferred to 45% of AV nodal myocytes by intracoronary catheterization. A limited inflammatory response was noted after adenoviral infection, but there was no detectable effect on AV nodal function from the inflammation or from reporter gene transfer. Other studies have shown that the use of first-generation adenoviruses (those with E1 deletions) leads to intense inflammation and loss of transgene expression 20 30 days after infection¹³. When used at high concentrations (much greater than those in this study), adenovirus vectors are also associated with endothelial damage, arterial thrombosis, thrombocytopenia, anemia, hepatitis, and death¹⁴⁻¹⁷. Wild-type adenoviruses have also been implicated in the development of myocarditis and idiopathic cardiomyopathy¹⁸. Since this study used a relatively low concentration of virus and looked at phenotypic changes early after gene transfer, these limitations did not affect the findings reported here.

This study is the first report of intracoronary site-specific gene transfer, as well as the first use of gene therapy to treat cardiac arrhythmias. We demonstrate that overexpression of an inhibitory component of the β-adrenergic signaling pathway suppresses AV nodal conduction, and also document the absence of electrophysiological changes after adenovirus-mediated transfer of a reporter gene. In summary, our research provides proof of the principle that in vivo gene transfer can modify the cardiac electrical substrate and lays the groundwork for future investigations to treat common arrhythmias.

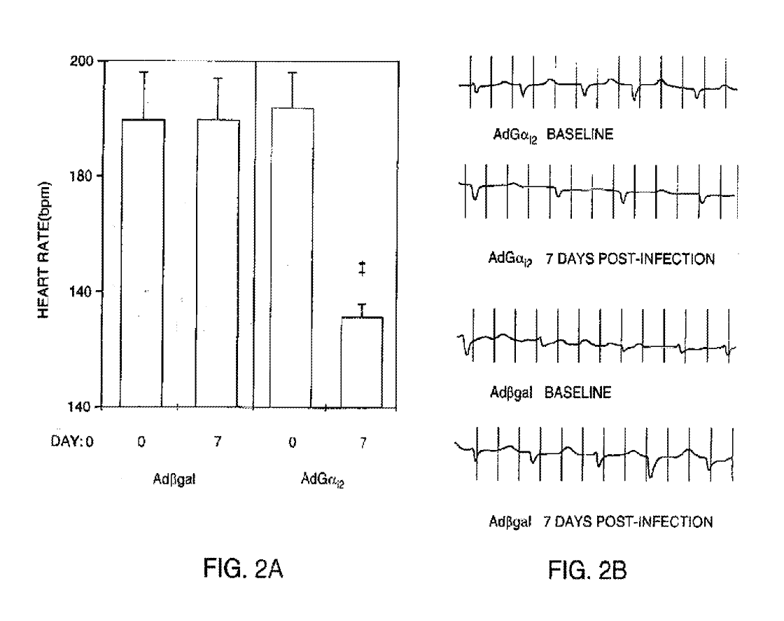

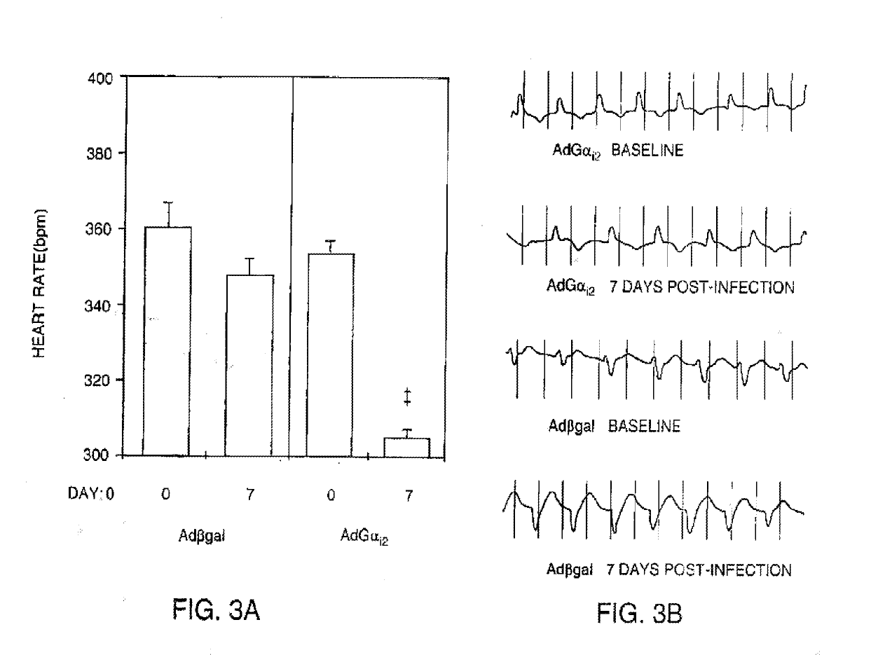

FIGS. 2A-B and 3A-B are explained in more detail as follows. Reduction in heart rate during atrial fibrillation after Gαᵢ₂ gene transfer. In the drug-free state, Gαᵢ₂ overexpression reduces ventricular rate by 20% during atrial fibrillation. No difference in heart rate is observed after Adβgal exposure. After infusion of epinephrine (1 mg, IV), the relative effect of Gαᵢ₂ overexpression persists (.dagger-dbl. p=0.005).

### Example 4

### Heart Rate Control During Atrial Fibrillation

The present example shows conduction slowing and increased refractoriness.

Atrial fibrillation affects more than 2 million people in the United States, including 5 10% of people over the age of 65 and 10 35% of the 5 million patients with congestive heart failure. Treatment strategies for AF include antiarrhythmic therapy to maintain sinus rhythm or ventricular rate control and anticoagulation. Although appealing, the maintenance of sinus rhythm is often unsuccessful. Within 1 year of conversion to sinus rhythm, 25 50% of patients revert to AF in spite of antiarrhythmic drug treatment. The usual clinical situation, then, is to maintain anticoagulation and ventricular rate control during chronic AF. The variable efficacy and frequent systemic adverse effects from rate controlling drugs motivated our development of animal models of gene transfer to control the heart rate in atrial fibrillation.

In porcine models of acute and chronic atrial fibrillation (AF), animals underwent coronary catheterization to deliver recombinant adenovirus to the atrioventricular nodal region of the heart. Immediately prior to catheterization, female domestic swine (30 40 kg) received sustained release diltiazem 180 mg, aspirin 325 mg and sildenafil 25 mg orally, and a mixture of ketamine 100 mg and acepromazine 4 mg intramuscularly. (For uniformity, the same pretreatment regimen, except administration of sildenafil, was used for all procedures to control for any effect these agents might have on the baseline EP measurements.) After sedation, anesthesia was induced with 5 10 ml of intravenous sodium pentothal 2.5% solution and maintained with inhaled isoflurane 2% in oxygen. The right carotid artery, right internal jugular vein and right femoral vein were accessed by sterile surgical technique, and introducer sheaths were inserted into each vessel. After baseline EP study, the right coronary artery was catheterized via the right carotid artery, using a 7 Fr. angioplasty guiding catheter. The AV nodal branch was selected with a 0.014″ guide wire, over which a 2.7 Fr. infusion catheter was inserted into the AV nodal artery. The following solutions were infused through the catheter: 10 ml of normal saline (NS) containing 5 μg of VEGF₁₆₅ and 200 μg of nitroglycerin over 3 minutes, 1 ml of normal saline containing 7.5×10⁹ pfu of AdGᵢ or Adβgal and 20 μg of nitroglycerin over 30 seconds, and 2 ml of normal saline over 30 seconds. After recovery from anesthesia, the animals received usual care and no additional medication. After one week, repeat EP evaluation was performed; the animals were sacrificed, and the organs were removed for histological evaluation.

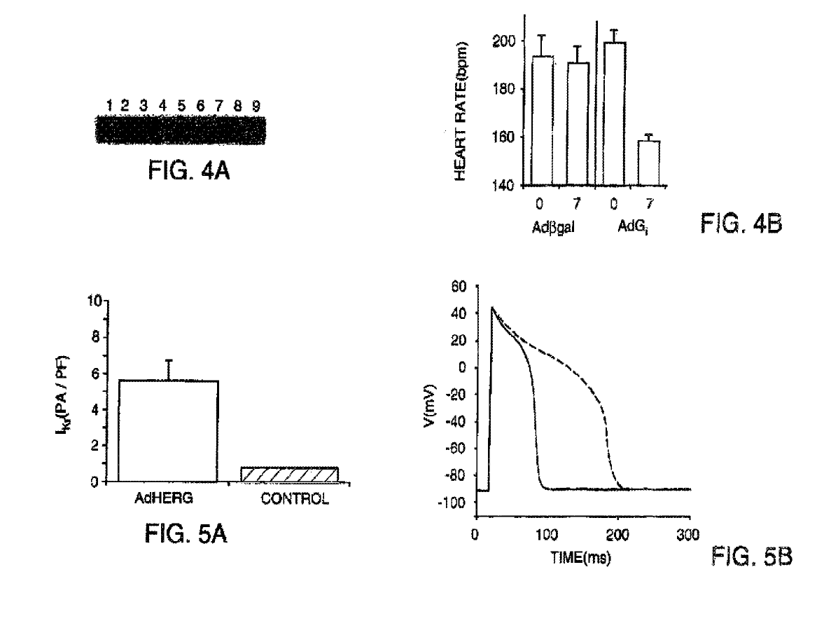

Oral treatment with sildenafil and infusion of VEGF, nitroglycerin and calcium-free solutions served to increase microvascular permeability and thus increase the efficiency of gene transfer. Using this delivery method, Western blot analysis demonstrated 600% overexpression of Gαᵢ₂ in the AdGᵢ group when compared to untreated or Adβgal-treated controls (FIG. 4A, p=0.01). The Adβgal-treated animals did not have significant differences in Gαᵢ₂ expression when compared to controls.²

After gene transfer, the heart rate was determined at the 1 week follow-up EP study for animals with acutely-induced AF, and heart rate was determined daily for animals with chronic AF. The acute AF model emulates the human condition of paroxysmal AF. In the acute AF model, Heart rate during acutely induced atrial fibrillation was decreased by 20% in the AdGi-treated animals and unchanged in the Adβgal-treated animals when compared to the untreated state (FIG. 4B, p=0.005 for AdGi and p=NS for Adβgal compared to baseline).² In the chronic AF model, heart rate in the AdGi group decreased by 35% 7-10 days after gene transfer. There was no change in heart rate in the Adβgal group. This example shows that Gαᵢ₂ overexpression is capable of reducing heart rate by 20-35% in acute and chronic models of AF. By comparison, currently available drug therapies reduce heart rate by 15-30%, but treatment is often limited by systemic side effects.¹

FIGS. 4A-B are explained in more detail as follows. FIG. 4A. Western blot of AV nodal tissue demonstrates Gaᵢ₂ overexpression in the AdGᵢ infected animals. Lane 1 is 10 mg of Gai2 control. Lanes 2, 4, 6, 8 are from Adbgal-infected animals and lanes 3, 5, 7, 9 are from AdGᵢ-infected animals. Analysis of the bands shows a 5.+−0.1-fold increase in Gaᵢ₂ content in the AdGᵢ animals relative to the Adbgal-infected controls. FIG. 4B. Analysis of heart rate before and 7 days after gene transfer. AdGi gene transfer reduces ventricular rate by 20% during atrial fibrillation (p=0.005). No difference in heart rate was observed after Adbgal exposure.

### Example 5

### Treatment of Polymorphic Ventricular Tachycardia in Congestive Heart Failure or the Long QT Syndrome

Sudden death in patients with congestive heart failure is a common clinical occurrence. In most studies, roughly half of all heart failure deaths were sudden in nature. Often, the associated arrhythmia is polymorphic ventricular tachycardia (VT) leading to ventricular fibrillation and death. The type of VT seen in these patients is similar to that observed in patients with the congenital long QT syndrome. Studies of animal models have documented the similarities between these two diseases on a tissue and cellular level. In both conditions, heterogeneous increases in the action potential duration (APD) have been a consistent finding. In heart failure, the APD prolongation correlates with downregulation of several potassium currents: the transient outward current Iₜₒ, the inward rectifier current I_{K1}, and the delayed rectifier currents I_{Ks} and lₖᵣ. In the long QT syndrome, prolongation of the action potential correlates with mutation in one of the potassium or sodium channel genes. Either condition disrupts the balance of inward and outward currents, predisposing the patient to malignant ventricular arrhythmias. This balance can be restored by gene transfer-induced overexpression of potassium channels.

In a guinea pig model, animals underwent surgical injection of AdHERG and then were followed for changes in APD and QT³ Adult guinea pigs (200-250 g) received metafane anesthesia. The abdomenal wall was incised in sterile surgical fashion. The diaphragm was fixated with forceps in incised in an anterior-posterior direction. The pericardium was fixated and opened. The heart was fixated, and 0.15 ml of AdHERG containing solution was injected into multiple sites in the left ventricular free wall. The incisions were closed and the animal was allowed to recover. After 3 days, the animals were sacrificed and the cardiac myocytes were enzymatically isolated. Using conventional patch clamp methods, APD and ion channel currents were measured. In comparison to control animals, AdHERG-infected animals exhibited a 7-fold increase in Iₖᵣ outward current and a 50% reduction in APD. See FIGS. 5A-B.³

FIGS. 5A-B are explained in more detail as follows. FIG. 5A. Comparison of Iₖᵣ current in the presence or absence of gene transfer-mediated overexpression of HERG. FIG. 5B. Photograph of an action potential tracing from a cell overexpressing HERG.

### Example 6

### Treatment of Atrial Fibrillation

The present example demonstrates therapeutic lengthening of the action potential.

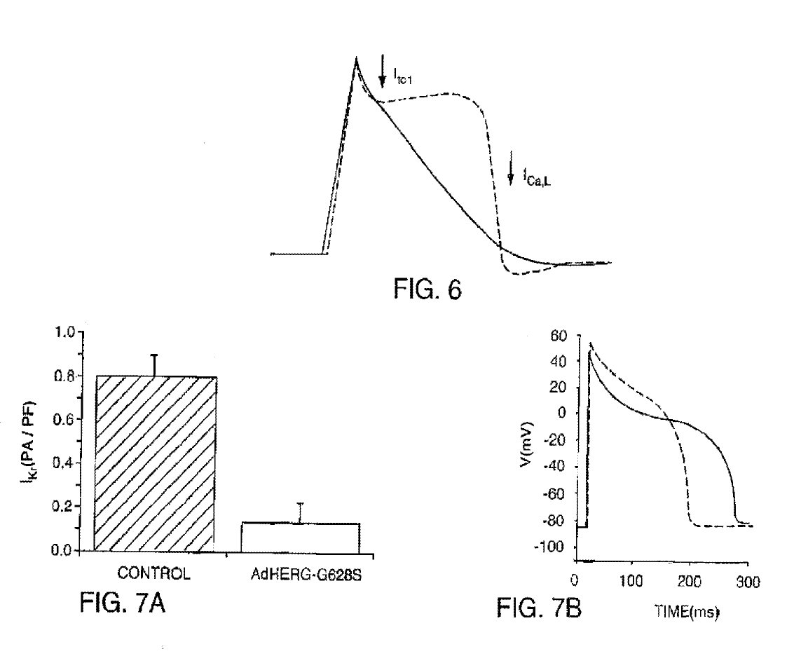

The cellular adaptive processes that occur with AF are completely different than those seen with heart failure. During sustained AF, there is a shortening of the APD and refractory period, essentially with loss of the plateau phase of the action potential (FIG. 6). Clinical and experimental studies have shown a 70% downregulation of the Ca²⁺ current, I_{caL}, and the transient outward current, Iₜₒ, to account for the observed changes in the AP morphology. The inward rectifier and adenosine/acetylcholine activated potassium currents (I_{K1} and I_{K,Ach}) are upregulated. The end result of these changes is an improved ability of the atrial myocytes to sustain the rapid and chaotic impulses characteristic of atrial fibrillation. This situation creates a cycle where the rapid rate causes a shortened refractory period which allows the continuation of the rapid rate, an idea that has been termed “AF begets AF”. The maladaptive nature of the ion channel alterations suggests that interrupting these changes on a molecular level is a potential treatment for AF.

FIG. 6 specifically shows changes in the atrial action potential after prolonged atrial fibrillation. Reduction in the transient outward current, Iₜₒ, and the 1-type calcium current, ICa,1 result in a decreased notch and plateau. A normal action potential is noted by the dashed line.

To evaluate the ability of potassium channel gene transfer to extend the plateau phase of the action potential, the guinea pig model illustrated in example 5 was used.³ Rather than injecting AdHERG to shorten the action potential, AdHERG-G628S was injected. This mutant reduced the intrinsic HERG and extended the plateau of the action potential in a controllable fashion. Iₖᵣ current density was reduced by 80%, which caused a 17% increase in APD (FIGS. 7A-B).³ Observation of the action potential morphology shows that the increase in APD occurs by extension of the plateau phase of the action potential. When applied to atrial fibrillation, this extension of the action potential would have an effect similar to that of potassium channel blocking drugs and reduce the occurrence of atrial fibrillation. Since the gene transfer-mediated increase would be specific to the atria, it would eliminate the ventricular proarrhythmic effects caused by antiarrhythmic drugs.

FIGS. 7A-B are explained in more detail as follows. FIG. 7A shows comparison of Iₖᵣ current in the presence or absence of gene transfer-mediated overexpression of a dominant negative mutant of HERG. FIG. 7B. Photograph of an action potential tracing from a cell overexpressing the mutant HERG.

### Example 7

### Construction and Use of a Biopacemaker

Patients who suffer heart block or other cardiac conduction system disorders require placement of an electronic pacemaker to maintain adequate blood flow. While this treatment is standard practice (about 250,000 cardiac pacemakers are implanted annually in the U.S.), it is expensive ($45,000 10-year cost) and carries substantial risk (infection, pneumothorax, etc.). A potential application of several embodiments of the invention is to increase automaticity of focal regions in the sinus node, atria, atrioventricular node, His-Purkinje system or ventricles in order to replicate the activity of the native pacemaker.

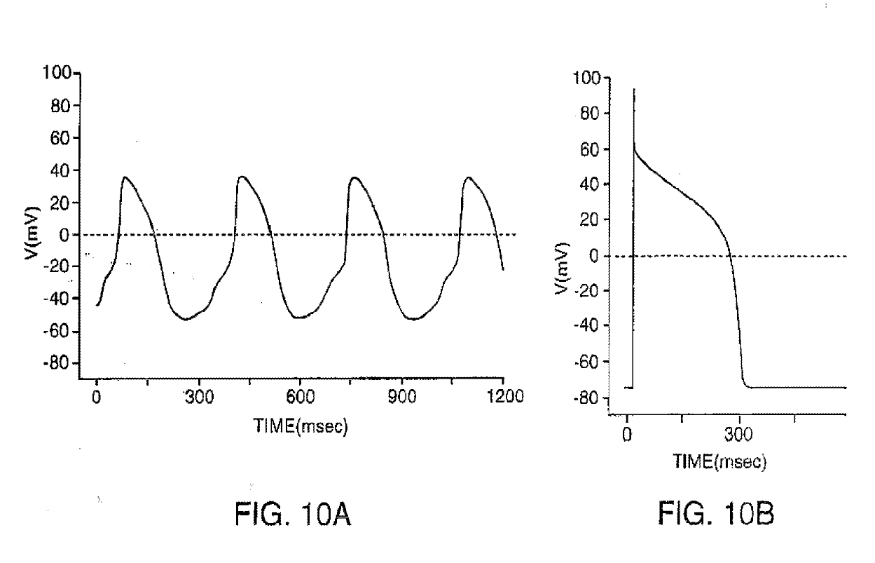

In proof of principle experiments, guinea pigs underwent surgical injection of AdcgiKir2.1AAA. After sufficient time for protein expression had elapsed, the cardiac myocytes were isolated and analyzed using conventional electrophysiological techniques. Adult guinea pigs (200 250 g) received metafane anesthesia. A left lateral thoracotomy was performed in sterile surgical fashion. The aorta was isolated. A cannula was passed through the LV apex into the proximal aorta. The aorta was cross-clamped and 0.15 ml of Kreb's solution containing AdKir2.1AAA was injected over 40 seconds. The cross clamp and cannula were removed; the incisions were closed, and the animal was allowed to recover. After 3 days, the animal was sacrificed. The heart was removed and cardiac myocytes were enzymatically isolated using conventional methods. Cells infected with the virus were identified by the presence of GFP fluorescence. No uninfected cells exhibited automaticity, while several AdcgiKir2.1AAA infected cells displayed spontaneous, regularly occurring action potentials. Examples of uninfected and infected cells are displayed in FIGS. 10A-B.

FIGS. 10A-B are explained in more detail as follows. FIG. 10A. Spontaneously occurring action potentials in guinea pig ventricular myocytes expression Kir2.1AAA. FIG. 10B Induced action potential from a control myocyte. No spontaneous action potentials were observed in control cells.

The following materials and methods were used as needed in the foregoing Examples.

Adenoviruses-1. Adβgal was a gift; the vector contained the E. coli lac Z gene driven by the human cytomegalovirus (CMV) immediate early promoter. AdGᵢ was constructed using a previously reported method¹⁹. The vector included the full-length rat Gαᵢ₂ gene driven by the CMV promoter. Virus stock expansion and quality control were performed as previously described⁴.

Gene Transfer Procedure. Immediately prior to catheterization, female domestic swine (30 40 kg) received sustained release diltiazem 180 mg, aspirin 325 mg and sildenafil 25 mg orally, and a mixture of ketamine 100 mg and acepromazine 4 mg intramuscularly. (For uniformity, the same pretreatment regimen, except administration of sildenafil, was used for all procedures to control for any effect these agents might have on the baseline EP measurements.) After sedation, anesthesia was induced with 5 10 ml of intravenous sodium pentothal 2.5% solution and maintained with inhaled isoflurane 2% in oxygen. The right carotid artery, right internal jugular vein and right femoral vein were accessed by sterile surgical technique, and introducer sheaths were inserted into each vessel. After baseline EP study (as described below), the right coronary artery was catheterized via the right carotid artery, using a 7 Fr. angioplasty guiding catheter. The AV nodal branch was selected with a 0.014″ guide wire, over which a 2.7 Fr. infusion catheter was inserted into the AV nodal artery. The following solutions were infused through the catheter: 10 ml of normal saline (S) containing 5 μg of VEGF₁₆₅ and 200 μg of nitroglycerin over 3 minutes, 1 ml of normal saline containing 7.5×10⁹ pfu of adenovirus and 20 μg of nitroglycerin over 30 seconds, and 2 ml of normal saline over 30 seconds. After recovery from anesthesia, the animals received usual care and no additional medication. After one week, repeat EP evaluation was performed; the animals were sacrificed, and the organs were removed for histological evaluation.

Electrophysiological Evaluation. Immediately prior to gene transfer and one week afterward, the animals underwent electrophysiological evaluation. A 5 Fr. steerable quadripolar EP catheter was placed through the right internal jugular vein into the high right atrium; a 5 Fr. non-steerable quadripolar EP catheter was placed through the same internal jugular vein into the right ventricle, and a 6 Fr. non-steerable quadripolar EP catheter was placed through the right femoral vein into the His bundle position. Baseline intracardiac electrograms were obtained, and electrocardiographic intervals were recorded. Following standard techniques, the AVNERP was measured by programmed stimulation of the right atrium with a drive train cycle length of 400 msec.

After baseline measurements were obtained, atrial fibrillation was induced by burst atrial pacing from a cycle length of 180 msec decrementing to 100 msec over 30 sec. Three attempts were made using this induction protocol. If no sustained atrial fibrillation was induced, the atria were paced at an output of 10 mA and a cycle length of 20 msec for 15 sec. The latter protocol reliably induced atrial fibrillation. The first episode of atrial fibrillation lasting longer than 12 sec was used for analysis. The median duration for atrial fibrillation episodes was 20 sec (range 14-120 sec). The heart rate was determined by measuring R-R intervals during the first 10 seconds of atrial fibrillation (average number of R-R intervals measured was 32 per recording). After conversion back to sinus rhythm, 1 mg of epinephrine was administered through the femoral venous sheath. Atrial fibrillation was re-induced in the presence of epinephrine (median episode duration 131 sec, range 20 sec-10 min), and the heart rate was again measured (average number of R-R intervals measured was 60 per recording). In the drug-free state, all episodes of atrial fibrillation terminated spontaneously. After epinephrine infusion, 4 episodes persisted for 10 minutes and were terminated by electrical cardioversion.

Histological Evaluation. After euthanasia, the heart and sections of lung, liver, kidney, skeletal muscle and ovary were removed and rinsed thoroughly in PBS. The atrial and ventricular septa were dissected from the heart and frozen to −80.degree. C. The remaining portions of the heart and other organs were sectioned, and alternating sections were used for gross or microscopic analysis. The sections for gross examination were fixed in 2% formaldehyde/0.2% glutaraldehyde for 15 minutes at room temperature, and stained for 6 hours at 37.degree. C. in PBS containing 1.0 mg/ml 5-bromo, 4-chloro, 3-indolyl-β-D-galactopy (X-gal), 15 mmol/L potassium ferricyanide, 15 mmol/L potassium ferrocyanide and 1 mmol/L MgCl₂. After staining, the slices were fixed with 2% formaldehyde/0.2% glutaraldehyde in PBS at 4.degree. C. overnight. The sections for microscopic analysis were embedded in paraffin, cut to 7 μm thickness, stained with X-gal solution as above and counterstained with Hematoxylin and eosin stains using traditional methods. β-galactosidase expression in the AV node was quantified by counting 100 cells in randomly chosen high-power fields of microscopic sections through the region.

Western Blot Analysis of Gαᵢ₂ Expression. To quantify Gαᵢ₂ gene expression, Western blot analysis of Gαᵢ₂ protein expression was performed on cytosolic extracts of frozen AV nodal tissue (Novex System). Samples were normalized for protein content, and SDS-polyacrylamide gel electrophoresis of the normalized samples was performed on 4 12% gradient gels. Proteins were then transferred to nitrocellulose membranes (30V, 1 hr). Detection of protein was performed by sequential exposure to Western Blocking Reagent (Boehringer Mannheim), a mouse monoclonal antibody against Gai2 (Neomarkers, 1 ug/ml, 2 hours), and goat-anti-mouse secondary antibody conjugated with horseradish peroxidase (NEN, 1: 10000, 30 min). Bands were detected with the enhanced chemiluminescence assay (Amersham) and quantified using the Quantity One software package (BioRad).

Statistical Analysis. The data are presented as mean.+−.s.e.m. Statistical significance was determined at the 5% level using the student's t test and repeated measures ANOVA, where appropriate.

The following materials and methods were specifically employed in Examples 4-6, above.

Adenovirus vectors-II. Adfβgal, AdGi, AdHERG, and AdHERG-G628S are recombinant adenoviruses encoding β-galactosidase, wild-type Gαᵢ₂, wild-type HERG, and HERG-G628S—a mutant of HERG found in some long QT syndrome patients. Gαᵢ₂ is the second isoform of the alpha-subunit of the inhibitory G protein, and HERG is a potassium channel. Expression of the mutant channels reduces the intrinsic current of the respective channel, and overexpression of the wild-type channel increases the intrinsic current. AdegiKir2.1AAA is a bicistronic adenoviral construct with enhanced GFP and Kir2.1AAA genes connected by an IRES sequence. By use of the IRES sequence, a single ecdysone promoter is capable of driving expression of both genes. The Kir2.1AAA mutant replaces GYG in the pore region with AAA, causing dominant negative suppression of Kir2.1.

All of the adenoviruses were created using standard methods. For Adβgal and AdGi, the CMV immediate-early promoter was used to drive gene expression, and for AdHERG, AdHERG-G628S and AdegiKir2.1AAA expression was driven by the ecdysone promoter system. Any promoter capable of driving expression of the transgene would be suitable under most circumstances. Virus stocks were maintained in phosphate buffered saline with 10% glycerol and 1 mM MgCl₂. Virus quality control included wild-type virus assay, infectious titre measurement by plaque assay, and transgene expression measurement by Western blot and functional assay appropriate to the specific gene.

See also the PCT application PCT/US98/23877 to Marban E., herein incorporated by reference, for additional disclosure relating to polynucleotides used in accord with embodiments of the present invention.

The following references (referred to by number throughout the text with the exception of Examples 46) are specifically incorporated herein by reference.
1. MacMahon, S., Collins, R., Peto, R., Koster, R. & Yusuf, S. Effect of prophylactic lidocaine in suspected acute myocardial infarction: an overview of results from the randomized, controlled trials. JAMA 260, 1910 1916 (1988).
2. Echt, D. et al. Mortality and morbidity in patients receiving encamide, flecamide, or placebo. N Engl J Med 324, 781 788 (1991).
3. Waldo, A. et al. Effect of d-sotalol on mortality in patients with left ventricular dysfunction after recent and remote myocardial infarction. Lancet 348, 7 12 (1996).
4. Donahue, J. K., Kikkawa, K., Johns, D., Marban, E. & Lawrence, J. Ultrarapid, highly efficient viral gene transfer to the heart. Proc Natl Acad Sci USA 94, 4664 4668 (1997).
5. Donahue, J. K., Kikkawa, K., Thomas, A. D., Marban, E. & Lawrence, J. Acceleration of widespread adenoviral gene transfer to intact rabbit hearts by coronary perfusion with low calcium and serotonin. Gene Therapy 5, 630 634 (1998).
6. Wu, H. M., Huang, Q., Yuan, Y. & Granger, H. J. VEGF induces NO-dependent hyperpermeability in coronary venules. Am J Physiol 271, H2735H2739 (1996).
7. Muhlhauser, J. et al. Safety and efficacy of in vivo gene transfer into the porcine heart with replication-deficient, recombinant adenovirus vectors. Gene Therapy 3, 145 153 (1996).
8. French, B., Mazur, W., Geske, R. & Bolli, R. Direct in vivo gene transfer into porcine myocardium using replication-deficient adenoviral vectors. Circulation 90, 2414 2424 (1994).
9. Kass-Eisler, A. et al. Quantitative determination of adenovirus-mediated gene delivery to rat cardiac myocytes in vitro and in vivo. Proc Natl Acad Sci USA 90, 11498 11502 (1993).
10. Kass-Eisler, A. et al. The Impact of Developmental Stage, Route of Administration and the Immune System on Adenovirus-Mediated Gene Transfer. Gene Therapy 1, 395 402 (1994).
11. Eschenhagen, T. G proteins and the heart. Cell Biol Int 17, 723 749 (1993).
12. Dessauer, C., Posner, B. & Gilman, A. Visualizing signal transduction: receptors, G-proteins, and adenylate cyclases. Clin Sci (Colch) 91, 527 537 (1996).
13. Quinones, M. et al. Avoidance of immune response prolongs expression of genes delivered to the adult rat myocardium by replication defective adenovirus. Circulation 94, 1394 1401 (1996).
14. Channon, K. et al. Acute host-mediated endothelial injury after adenoviral gene transfer in normal rabbit arteries: impact on transgene expression and endothelial function. Circ Res 82, 1253 1262 (1998).
15. Lafont, A. et al. Thrombus generation after adenovirus-mediated gene transfer into atherosclerotic arteries. Hum Gene Ther 9, 2795 2800 (1998).
16. Cichon, G. et al. Intravenous administration of recombinant adenoviruses causes thrombocytopenia, anemia, and erythroblastosis in rabbits. J Gene Med 1, 360 371 (1999).
17. Marshall, E. Gene therapy death prompts review of adenovirus vector. Science 286, 2244 2245 (1999).
18. Pauschinger, M. et al. Detection of adenoviral genome in the myocardium of adult patients with idiopathic left ventricular dysfunction. Circulation 99, 1348 1354 (1999).
19. Akhter, S. et al. Restoration of beta-adrenergic signaling in failing cardiac ventricular myocytes via adenoviral-mediated gene transfer. Proc Natl Acad Sci USA 94, 12100 12105 (1997).

The following references are also incorporated by reference. Each reference is referred to by number only in Examples 46, above.
1. Khand A, Rankin A, Kaye G, Cleland J. Systematic review of the management of atrial fibrillation in patients with heart failure. Eur Heart J 2000; 21: 614 632.
2. Donahue J K, Heldman A H, Fraser H, McDonald A D, Miller J M, Rade J J, Eschenhagen T, Marban E. Focal Modification of Electrical Conduction in the Heart by Viral Gene Transfer. Nature Med 2000; 6:1395 1398.
3. Hoppe U C, Marban E, Johns D C. Distinct gene-specific mechanisms of arrhythmia revealed by cardiac gene transfer of two long QT disease genes, HerG and KCNE1. Proc Nat Acad Sci 2001; 98:5335 5340.

All references are incorporated herein by reference.

### Biological Pacemaker

As discussed above, we now provide gene transfer and cell administration methods to induce and/or modulate the activity of an endogenous or induced cardiac pacemaker function. In particular, several embodiments of the invention provide for the creation of genetically-engineered pacemakers using gene therapy as an alternative and/or supplement to implantable electronic pacemakers. In preferred aspects of several embodiments of the invention, quiescent heart muscle cells are converted into pacemaker cells by in vivo viral gene transfer. Cardiac contraction and/or an electrical property of those converted cells then may be modulated in accordance with several embodiments of the invention.

More particularly, in a first aspect of several embodiments of the invention, methods may be employed to induce a pacemaker function (cardiac contraction) in myocardial cells that have not been exhibiting such properties, e.g., quiescent myocardial cells that exhibit no, little or inappropriate firing rate. Preferably, the administration induces or otherwise causes the treated cardiac cells to generate spontaneous repetitive electrical signals, e.g., for myocardial cells that exhibited little (firing rate of about 20, 15, 10, 5 per minute or less) or no firing rate, the frequency of the firing rate or electrical signal output will preferably increase to a detectable level, particularly a firing rate or electrical signal output increase of at least about 3, 5, 10, 15, 20 or 25 percent after the administration.

In a further aspect, several embodiments of the invention are employed to modulate or “tune” the existing firing rate of myocardial cells. In this aspect, excessive ventricular pacing may be decreased to a decreased frequency or firing rate, or ventricular pacing rates that are too low may be increased to a desired level. This embodiment is particularly useful to modulate the effect achieved with an implanted (electronic) pacemaker effect to provide an optimal heart rate for a patient. Further, several embodiments of the invention have the advantage of maintaining the responsiveness of tissues being treated to endogenous neuronal or hormonal inputs.

Significantly, several embodiments of the invention may be employed to augment or supplement the effect of an implanted electronic pacemaker. That is, a mammal that has an implanted electronic pacemaker may be treated in accordance with several embodiments of the invention, e.g., a composition such as a polynucleotide or modified cells may be administered to the mammal to further modulate cardiac firing rate that is provided by the implanted electronic device. By such a combined approach, a precise and optimal firing rate can be achieved. Additionally, the composition can be administered to a site in the mammalian heart that is remote from the electronic pacemaker, e.g. the composition administration site being at least about 0.5, 1, 2, 3, 4 or 5 centimeters from the implanted electronic device, to thereby provide a pacemaker effect through a greater area of cardiac tissue.

Several embodiments of the invention may suitably be employed to modulate a treated subject's cardiac firing rate to within about 15 or 10 percent of a desired firing rate, more preferably about 8, 5, 4, 3 or 2 percent of a desired firing rate value.

Several embodiments of the invention include administration of a polynucleotide that codes for, particularly a polynucleotide that is introduced into pacemaker cells such as in the sinoatrial node of a mammalian heart, or administration of inducible cells such as pacemaker cells created from stem cells or converted from electrically quiescent cells, or other cells adapted to generate rhythmic contraction or cardiologic excitation. As discussed above, preferred methods involve administering a therapeutically effective amount of at least one polynucleotide or modified cell capable of modulating heart contraction (firing rate). Polynucleotides and modified cells also are preferred therapeutic compositions for administration in accordance with several embodiments of the invention due to the ease of localized administration of those agents within a targeted region of cardiac tissue.

Suitable compositions for administration to modulate firing rate of myocardial cells also can be readily identified by simple testing, e.g., a candidate agent such as a polynucleotide can be administered to myocardial cells to determine if the administered agent modulates firing rate relate to control myocardial cells (same cells that are untreated with agent) as determined for instance by a standard electrophysiological assay as such assay is defined below. Particularly preferred polynucleotides for administration are dominant-negative constructs. These constructs, include but are not limited to, for example, Kir2 constructs, HCN constructs, mutants, fragments and combinations thereof.

In a preferred embodiment of the invention, somatic gene transfer of a dominant-negative Kir2 constructs produces spontaneous pacemaker activity in the ventricle, resulting in the creation of biological pacemakers by localized genetic suppression Of I_{K1} of dormant pacemakers present within the working myocardium.

For instance, a Kir2 dominant-negative construct can be produced by, for example, replacement of amino acid residues in the pore region of Kir2.1 by alanines (GYG₁₄₄₋₁₄₆-AAA, or Kir2.1AAA). Such a dominant-negative construct can suppress current flux when co-expressed with wild-type Kir2.1. Incorporation of at least about one single mutant subunit within the tetrameric Kir channel can be sufficient to knock out function. The dominant-negative construct, for example, Kir2.1AAA can be packaged into a bicistronic vector and injected into the left ventricular cavity of guinea pigs. Preferably, in a localized area such as an area of approximately 1 cubic cm, at least about 10% of ventricular myocytes are transduced, more preferably at least about 20% ventricular myocytes are transduced, most preferably at least about 30%, 40% or 50% ventricular myocytes are transduced. Measurement of I_{K1} and calcium currents are conducted as described in detail in the examples which follow.

The above activities of transduced myocytes are compared to control ventricular myocytes spontaneous activity as described in the Examples which follow. It is desirable for the transduced myocytes to exhibit spontaneous activity representative of pacemaker cells, such as the early embryonic heart cells which possess intrinsic pacemaker activity or the normal pacemaker cells of the sinoatrial node.

In another preferred embodiment, the invention provides for antisense therapeutic molecules which inhibit the expression of Kir2 gene products. In therapeutic applications oligonucleotides have been used successfully to block translation in vivo of specific mRNAs thereby preventing the synthesis of proteins which are undesired or harmful to the ce/lorganism. This concept of oligonucleotide mediated blocking of translation is known as the “antisense” approach. Mechanistically, the hybridizing oligonucleotide is thought to elicit its effect by either creating a physical block to the translation process or by recruiting cellular enzymes that specifically degrade the mRNA part of the duplex (RNaseH).

To be useful in an extensive range of applications, oligonucleotides preferably satisfy a number of different requirements. In antisense therapeutics, for instance, a useful oligonucleotide must be able to penetrate the cell membrane, have good resistance to extra and intracellular nucleases and preferably have the ability to recruit endogenous enzymes like RNaseH. In DNA-based diagnostics and molecular biology other properties are important such as, e.g., the ability of oligonucleotides to act as efficient substrates for a wide range of different enzymes evolved to act on natural nucleic acids, such as e.g. polymerases, kinases, ligases and phosphatases. Oligonucleotides used as antisense therapeutic molecules need to have both high affinity for its target mRNA to efficiently impair its translation and high specificity to avoid the unintentional blocking of the expression of other proteins.

In particular, it is preferred to have antisense oligonucleotides which inhibit the expression of at least about one component that makes up the Kir2 channel, or enough components that make up the Kir2 channels, to specifically suppress Kir2 channels sufficient to unleash pacemaker activity in ventricular myocytes, as measured by the partial suppression or the absence of strongly-polarizing I_{K1}.

Other administration protocols may be employed. For example, the administered polynucleotide may function as a decoy, where the polynucleotide is introduced to targeted cells or genes by any convenient means, wherein activation of a gene is interrupted e.g. by diverting transcription factors to the decoy molecule. More particularly, a decoy can be employed to effectively inhibit expression of a Kir2 channel component. As used herein, the term “decoy molecule” or other similar term includes reference to a polynucleotide that codes for a functionally inactive protein or the protein itself which competes with a functionally active protein and thereby inhibits the activity promoted by the active protein. A Kir2 decoy protein can thus acts as a competitive inhibitor to wild type Kir2 proteins thereby inhibiting formation of Kir2 wild type channels and resulting in suppression of inward rectifier potassium current (I_{K1})

Preferably, the dominant negative constructs must be durable, e.g., long-lasting, such as for months for years and regionally specific, e.g., only target the desired tissue and specifically act on the mechanism of choice, for example, a Kir2 dominant-negative construct specifically suppresses Kir2 channels sufficient to unleash pacemaker activity in ventricular myocytes, as measured by the absence of strongly-polarizing I_{K1}

Another example of a dominant-negative construct for use in accordance with several embodiments of the invention is an HCN construct, such as an HCN1 construct. Particularly, in such a construct, the critical residues GYG in the pore have been converted to AAA (to create HCN1-AAA), that is capable of suppressing the normal HCN-encoded pacemaker currents.

More particularly, as further shown in the examples which follow, the functional importance of the GYG selectivity motif in pacemaker channels was evaluated by replacing that triplet in HCN1 with alanines (GYG₃₆₅₋₃₆₇AAA). HCN1-AAA did not yield functional currents; co-expression of HCN1-AAA with WT HCN1 suppressed normal channel activity in a dominant-negative manner (55.2.+−3.2, 68.3.+−4.3, 78.7.+−1.6, 91.7.+−0.8, 97.9.+−0.2% current reduction at −140 mV for WT:AAA ratios of 4:1, 3:1, 2:1, 1:1 and 1:2, respectively) without affecting gating (steady-state activation, activation and deactivation kinetics) or permeation (reversal potential) properties. Statistical analysis reveals that a single HCN channel is composed of four monomeric subunits. Interestingly, HCN 1-AAA also inhibited HCN2 in a dominant-negative manner with the same efficacy—It is thus believed that the GYG motif is a critical determinant of ion permeation for HCN channels, and that HCN1 and HCN2 readily coassemble to form heterotetrameric complexes.

As indicated above, through the co-assembly of different HCN isoforms, endogenous HCN activity (e.g. activation thresholds and expressed current amplitudes) can be modulated in both directions thereby enabling effective modulation of cardiac pacing or firing rate, such as within a preferred range of a desired value as discussed above.

Several embodiments of the invention are generally compatible with one or a combination of suitable polynucleotide administration routes including those intended for in vivo or ex vivo cardiac use. There is understanding in the field that cardiac tissue is especially amenable to gene transfer techniques. See e.g., Donahue, J. et al. (1998) Gene Therapy 5: 630; Donahue, J. et al. PNAS (USA) 94: 4664 (disclosing rapid and efficient gene transfer to the heart); Akhter, S. et al. (1997) PNAS (USA) 94: 12100 (showing successful gene transfer to cardiac ventricular myocytes); all herein incorporated by reference and references cited therein. Preferred nucleic acid delivery methods are disclosed in U.S. Pat. No. 6,376,471, herein incorporated by reference.

Further preferred administration routes according to several embodiments of the invention involve introducing the polynucleotide into cardiac tissue and expressing same sufficient to detectably decrease heart rate as determined by a standard electrocardiogram (ECG) recording. Preferably, the decrease in heart rate is at least about 5% relative to baseline.

Several embodiments of the invention are highly flexible and can be used with one or a combination of polynucleotides, preferably those encoding at least one therapeutic heart protein.

In addition to the preferred polynucleotides discussed above, suitable polynucleotides for administration in accordance with several embodiments of the invention include, but are not limited to, those encoding at least one ion channel protein, gap junction protein, G protein subunit, connexin; or functional fragment thereof. More preferred are polynucleotides encoding a K channel subunit, Na channel subunit, Ca channel subunit, an inhibitory G protein subunit; or a functional fragment thereof. Additionally preferred polynucleotides will encode one, two or three of such proteins (the same or different).

By the phrase “fragment”, “function fragment” or similar term is meant a portion of an amino acid sequence (or polynucleotide encoding that sequence) that has at least about 70%, preferably at least about 80%, more preferably at least about 95% of the function of the corresponding full-length amino acid sequence (or polynucleotide encoding that sequence). Methods of detecting and quantifying functionality in such fragments are known and include the standard electrophysiological assays disclosed herein.

Suitable polynucleotides for practicing several embodiments of the invention can be obtained from a variety of public sources including, but not limited to, GenBank (National Center for Biotechnology Information (NCBI)), EMBL data library, SWISS-PROT (University of Geneva, Switzerland), the PIR-International database; and the American Type Culture Collection (ATCC) (10801 University Boulevard, Manassas, Va. 20110-2209). See generally Benson, D. A. et al. (1997) Nucl. Acids. Res. 25:1 for a description of Genbank.

More particular polynucleotides for use with embodiments of the present invention are readily obtained by accessing public information from GenBank. For example, in one approach, a desired polynucleotide sequence is obtained from GenBank. The polynucleotide itself can be made by one or a combination of routine cloning procedures including those employing PCR-based amplification and cloning techniques. For example, preparation of oligonucleotide sequence, PCR amplification of appropriate libraries, preparation of plasmid DNA, DNA cleavage with restriction enzymes, ligation of DNA, introduction of DNA into a suitable host cell, culturing the cell, and isolation and purification of the cloned polynucleotide are known techniques. See e.g., Sambrook et al. in Molecular Cloning: A Laboratory Manual (2d ed. 1989); and Ausubel et al. (1989), Current Protocols in Molecular Biology, John Wiley & Sons, New York.

Table 1 below, references illustrative polynucleotides from the GenBank database for use with embodiments of the present invention.

TABLE 1

| Polynucleotide | GenBank Accession No. |
|---|---|
| Kir 2.1 potassium channel | XM028411¹ |
| HERG potassium channel | XM004743 |
| Connexin 40 | AF151979 |
| Connexin 43 | AF151980 |
| Connexin 45 | U03493 |
| Na channel alpha subunit | NM000335 |
| Na channel beta-1 subunit | NM001037 |
| L-type Ca channel alpha-1 subunit | AF201304 |
| HCN1 | NM010408; |
| | AF247450; |
| | AF064876 |

¹An additional polynucleotide for use with the present invention is the Kir 2.1 AAA mutant, which is wild-type Kir 2.1 with a substitution mutation of AAA for GFG in position 144-146.

Additional polynucleotides for use with several embodiments of the invention have been reported in the following references: Wong et al. Nature 1991; 351(6321):63 (constitutively active Gi2 alpha);) De Jongh K S, et al. J Biol Chem 1990 Sep. 5; 265(25):14738 (Na and Ca channel beta subunits); Perez-Reyes, E. et al. J Biol Chem 1992 Jan. 25; 267(3):1792; Neuroscientist 2001 February; 7(1):42 (providing sodium channel beta subunit information); Isom, LL. Et al. Science 1992 May 8; 256(5058):839 (providing the beta 1 subunit of a brain sodium channel); and Isom, LL. Et al. (1995) Cell 1995 Nov. 3; 83(3):433 (reporting beta 2 subunit of brain sodium channels), all herein incorporated by reference.

Further polynucleotides for use with several embodiments of the invention have been reported in PCT application number PCT/US98/23877 to Marban, E., herein incorporated by reference.

See also the following references authored by E. Marban: J. Gen Physiol. 2001 August; 118(2):171-82; Circ Res. 2001 Jul. 20; 89(2):160-7; Circ Res. 2001 Jul. 20; 89(2):101; Circ Res. 2001 Jul. 6; 89(1):33-8; Circ Res. 2001 Jun. 22; 88(12):1267-75; J. Biol. Chem. 2001 Aug. 10; 276(32):30423-8; Circulation. 2001 May 22; 103(20):2447-52; Circulation. 2001 May 15; 103(19):23614; Am J Physiol Heart Circ Physiol. 2001 June; 280(6):H2623-30; Biochemistry. 2001 May 22; 40(20):6002-8; J. Physiol. 2001 May 15; 533(Pt 1):127-33; Proc Natl Acad Sci USA. 2001 Apr. 24; 98(9):533540; Circ Res. 2001 Mar. 30; 88(6):570-7; Am J Physiol Heart Circ Physiol. 2001 April; 280(4):H1882-8; and J Mol Cell Cardiol. 2000 November; 32(11):1923-30, all herein incorporated by reference.

Further examples of suitable Ca channel subunits include beta 1, or alpha2-delta subunit from an L-type Ca channel. A preferred Na channel subunit is beta 1 or beta2. In some invention embodiments it will be useful to select Na and Ca channel subunits having dominant negative activity as determined by the standard electrophysiological assay described below. Preferably, that activity suppresses at least about 10% of the activity of the corresponding normal Na or Ca channel subunit as determined in the assay.

Particularly preferred constructs for administration in accordance with several embodiments of the invention also are disclosed in the examples which follow.

Also preferred is the inhibitory G protein subunit (“Gαᵢ₂”) or a functional fragment thereof, as a supplemental strategy to modulate pacemaker activity.

Several embodiments of the invention are broadly suited for use with gap junction proteins, especially those known or suspected to be involved with cardiac function. Particular examples include connexin 40, 43, 45; as well as functional fragments thereof. Further contemplated are polynucleotides that encode a connexin having dominant negative activity as determined by the assay, preferably a suppression activity of at least about 10% with respect to the corresponding normal connexin 40, 43, or 45. Conneixns may be particularly useful to induce/force stem cells or derived cardiomyocytes to form electrical couplings with quiescent heart tissue.

Also envisioned are mutations of such polynucleotides that encode dominant negative proteins (muteins) that have detectable suppressor activity. Encoded proteins that are genetically dominant typically inhibit function of other proteins particularly those proteins capable of forming binding complexes with the wild-type protein.

Additional polynucleotides of several embodiments of the invention encode essentially but not entirely full-length protein. That is, the protein may not have all the components of a full-length sequence. For example, the encoded protein may include a complete or nearly complete coding sequence (cds) but lack a complete signal or poly-adenylation sequence. It is preferred that a polynucleotide and particularly a cDNA encoding a protein of several embodiments of the invention include at least a complete cds. That cds is preferably capable of encoding a protein exhibiting a molecular weight of between about 0.5 to 70, preferably between about 5 and 60, and more preferably about 15, 20, 25, 30, 35, 40 or 50 kD. That molecular weight can be readily determined by suitable computer-assisted programs or by SDS-PAGE gel electrophoresis.

The polynucleotide and particularly the cDNA encoding the full-length protein can be modified by conventional recombinant approaches to modulate expression of that protein in the selected cells, tissues or organs.

More specifically, suitable polynucleotides can be modified by recombinant methods that can add, substitute or delete one or more contiguous or non-contiguous amino acids from that encoded protein. In general, the type of modification conducted will relate to the result of expression desired.

For example, a cDNA polynucleotide encoding a protein of interest such as an ion channel can be modified so as to overexpress that protein relative to expression of the full-length protein (e.g., control assay). Typically, the modified protein will exhibit at least 10 percent or greater overexpression relative to the full-length protein; more preferably at least 20 percent or greater; and still more preferably at least about 30, 40, 50, 60, 70, 80, 100, 150, or 200 percent or greater overexpression relative to the control assay.

As noted above, further contemplated modifications to a polynucleotide (nucleic acid segment) and particularly a cDNA are those which create dominant negative proteins.

In general, a variety of dominant negative proteins can be made by methods known in the field. For example, ion channel proteins are recognized as one protein family for which dominant negative proteins can be readily made, e.g., by removing selected transmembrane domains. In most cases, the function of the ion channel binding complex is substantially reduced or eliminated by interaction of a dominant negative ion channel protein.

Several specific strategies have been developed to make dominant negative proteins. Exemplary of such strategies include oligonucleotide directed and targeted deletion of cDNA sequence encoding the desired protein.

It is stressed that creation of a dominant negative protein is not synonymous with other conventional methods of gene manipulation such as gene deletion and antisense RNA. What is meant by “dominant negative” is specifically what is sometimes referred to as a “poison pill” which can be driven (e.g., expressed) by an appropriate DNA construct to produce a dominant negative protein which has capacity to inactivate an endogenous protein.

For example, in one approach, a cDNA encoding a protein comprising one or more transmembrane domains is modified so that at least 1 and preferably 2, 3, 4, 5, 6 or more of the transmembrane domains are eliminated. Preferably, the resulting modified protein forms a binding complex with at least one other protein and usually more than one other protein. As noted, the modified protein will inhibit normal function of the binding complex as assayed, e.g., by standard ligand binding assays or electrophysiological assays as described herein. Exemplary binding complexes are those which participate in electrical charge propagation such as those occurring in ion channel protein complexes. Typically, a dominant negative protein will exhibit at least 10 percent or greater inhibition of the activity of the binding complex; more preferably at least 20 percent or greater; and still more preferably at least about 30, 40, 50, 60, 70, 80, or 100 percent or greater inhibition of the binding complex activity relative to the full-length protein.

As a further illustration, a cDNA encoding a desired protein for use in the present methods can be modified so that at least one amino acid of the protein is deleted. The deleted amino acid(s) can be contiguous or non-contiguous deletions essentially up to about 1%, more preferably about 5%, and even more preferably about 10, 20, 30, 40, 50, 60, 70, 80, or 95% of the length of the full-length protein sequence.

Alternatively, the cDNA encoding the desired protein can be modified so that at least one amino acid in the encoded protein is substituted by a conservative or non-conservative amino acid. For example, a tyrosine amino acid substituted with a phenylalanine would be an example of a conservative amino acid substitution, whereas an arginine replaced with an alanine would represent a non-conservative amino acid substitution. The substituted amino acids can be contiguous or non-contiguous substitutions essentially up to about 1%, more preferably about 5%, and even more preferably about 10, 20, 30, 40, 50, 60, 70, 80, or 95% of the length of the full-length protein sequence.

Although generally less-preferred, the nucleic acid segment encoding the desired protein can be modified so that at least one amino acid is added to the encoded protein. Preferably, an amino acid addition does not change the ORF of the cds. Typically, about 1 to 50 amino acids will be added to the encoded protein, preferably about 1 to 25 amino acids, and more preferably about 2 to 10 amino acids. Particularly preferred addition sites are at the C- or N-terminus of the selected protein.

Preferred invention practice involves administering at least one of the foregoing polynucleotides with a suitable myocardium nucleic acid delivery system. In one embodiment, that system includes a non-viral vector operably linked to the polynucleotide. Examples of such non-viral vectors include the polynucleoside alone or in combination with a suitable protein, polysaccharide or lipid formulation.

As used herein, the term “operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

As used herein, the term “coding sequence” or a sequence which “encodes” a particular protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, “nucleic acid” sequence refers to a DNA or RNA sequence. The term also captures sequences that include any of the known base analogues of DNA and RNA such as, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudo-uracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, S-methoxyaminomethyl-2-thiour-acil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, 2-thiocytosine, and 2,6 diaminopurine.

As used herein, the term “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

As used herein, “promoter region” is used herein in its ordinary sense to refer to a DNA regulatory sequence to which RNA polymerase binds, initiating transcription of a downstream (3′ direction) coding sequence.

A particularly preferred myocardium nucleic acid system, especially when it is desirable to suppress a certain activity such as I_{K1} is described in U.S. Pat. No. 6,214,620 to D. C. Johns and E. Marban, the contents of which are hereby incorporated by reference in their entirety. Such a construct is controlled by use of an inducible promoter. Examples of such promoters, include, but not limited to those regulated by hormones and hormone analogs such as progesterone, ecdysone and glucocorticoids as well as promoters which are regulated by tetracycline, heat shock, heavy metal ions, interferon, and lactose operon activating compounds. For review of these systems see Gingrich and Roder, 1998, Ann. Rev. Neurosci., 21, 377-405, herein incorporated by reference. When using non-mammalian induction systems, both an inducible promoter and a gene encoding the receptor protein for the inducing ligand are employed. The receptor protein typically binds to the inducing ligand and then directly or indirectly activates transcription at the inducible promoter.

Additional suitable myocardium nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomegalovirus (CMV) promoter.

Additionally preferred vectors include viral vectors, fusion proteins and chemical conjugates. Retroviral vectors include moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England (1995); Geller, A. I. et al., Proc Natl. Acad. Sci.: U.S.:90 7603 (1993); Geller, A. J., et al., Proc Natl. Acad. Sci. USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)], all herein incorporated by reference.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are may be indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. Preferred in vivo or ex vivo cardiac administration techniques have already been described.

To simplify the manipulation and handling of the polynucleotides described herein, the nucleic acid is preferably inserted into a cassette where it is operably linked to a promoter. The promoter must be capable of driving expression of the protein in cells of the desired target tissue. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)), herein incorporated by reference, and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamasegene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618, herein incorporated by reference.

U.S. Published Patent Application US20020022259A1, herein incorporated by reference, also reports polynucleotide enhancer elements for facilitating gene expression in cardiac cells and differentiating stem cells to cardiomyocytes.

If desired, the polynucleotides of several embodiments of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988), herein incorporated by reference. See also, Feigner and Holm, Bethesda Res. Lab. Focus, 11 (2):21 (1989) and Maurer, R. A., Bethesda Res. Lab. Focus, 11(2):25 (1989), all herein incorporated by reference.

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Iniest., 90:626-630 (1992); and Rosenfeld, et al., Cell, 68:143-155 (1992), all herein incorporated by reference.

One preferred myocardicum delivery system is a recombinant viral vector that incorporates one or more of the polynucleotides therein, preferably about one polynucleotide. Preferably, the viral vector used in several embodiments of the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

Choice of a particular myocardium delivery system will be guided by recognized parameters including the condition being treated and the amount and length of expression desired. Use of virus vectors approved for human applications e.g., adenovirus are particularly preferred.

Reference herein to an electrophysiological assay is meant a conventional test for determining cardiac action potential (AP). See generally Fogoros RN. Electrophysiologic Testing Blackwell Science, Inc. (1999) for disclosure relating to performing such tests, herein incorporated by reference.

Specific reference herein to a “standard electrophysiological assay” is meant the following general assay.

1) providing a mammalian heart (in vivo or ex vivo),

2) contacting the heart with at least one suitable polynucleotide preferably in combination with an appropriate myocardium nucleic acid delivery system, or with modified cells as disclosed herein such as stem cells that have differentiated to cardiomyocytes,

3) transferring the polynucleotide or modified cells into the heart and under conditions which can allow expression of the encoded amino acid sequence; and

4) detecting modulation (increase or decrease) of at least one electrical property in the administered (e.g. transformed) heart e.g., at least one of conduction, ventricular response rate, firing rate and/or pulse rate, preferably firing rate or pulse rate, relative to a baseline value. As will be appreciated, baseline values will often vary with respect to the particular polynucleotide(s) chosen. Methods to quantify baseline expression or protein include western blot, quantitative PCR, or functional assays such as adenylate cyclase assay for inhibitory G proteins, patch clamp analysis for ion channel currents. Electrophysiology (EP) effects can be determined by measuring heart rate, conduction velocity or refractory period in vivo with EP catheters. Preferred rates of modulation are at least 1, 2, 3, 4, 5, 6, 7, 8, 9, or 10 percent difference from a baseline value. Greater increases or decreases from a baseline value also may be achieved e.g. an increase or decrease of heart rate or other measured property of at least about 12, 15, 20 or 25 percent relative to a baseline value. Methods according to embodiments of the invention can be performed in vitro or in situ.

Several embodiments of the invention include modifying the polynucleotide along lines discussed above sufficient to overexpress the encoded protein. Further preferred are methods in which the nucleic acid is modified to produce a dominant negative ion channel protein. The ion channel protein can be a voltage-gated (such as sodium, calcium, or potassium channel) or a ligand-gated ion channel. Additional disclosure relating to such channel proteins can be found in the discussion above and in U.S. Pat. No. 5,436,128, for instance.

Practice of several embodiments of the invention are broadly compatible with one or a combination of different administration (delivery) systems.

In particular, one suitable administration route involves one or more appropriate polynucleotide into myocardium. Alternatively, on in addition, the administration step includes perfusing the polynucleotide into cardiac vasculature. If desired, the administration step can further include increasing microvascular penneability using routine procedures, typically administering at least one vascular permeability agent prior to or during administration of the gene transfer vector. Examples of particular vascular permeability agents include administration of one or more of the following agents preferably in combination with a solution having less than about 500 micromolar calcium: substance P, histamine, acetylcholine, an adenosine nucleotide, arachidonic acid, bradykinin, endothelin, endotoxin, interleukin-2, nitroglycerin, nitric oxide, nitroprusside, a leukotriene, an oxygen radical, phospholipase, platelet activating factor, protamine, serotonin, tumor necrosis factor, vascular endothelial growth factor, a venom, a vasoactive amine, or a nitric oxide synthase inhibitor. A particular is serotonin, vascular endothelial growth factor (VEGF), or a functional VEGF fragment to increase the permeability.

Typical perfusion protocols in accord with several embodiments of the invention are generally sufficient to transfer the polynucleotide to at least about 10% of cardiac myocytes in the mammal. Infusion volumes of between from about 0.5 to about 500 ml are preferred. Also preferred are coronary flow rates of between from about 0.5 to about 500 ml/min. Additionally preferred perfusion protocols involve the AV nodal artery. Transformed heart cells, typically cardiac myocytes that include the polynucleotide are suitably positioned at or near the AV node.

Illustrative strategies for detecting modulation of transformed heart have been disclosed e.g., in Fogoros RN, supra, herein incorporated by reference. A preferred detection strategy is performing a conventional electrocardiogram (ECG). Modulation of cardiac electrical properties by use of several embodiments of the invention is readily observed by inspection of the ECG. Methods according to embodiments of the invention can be performed in vitro or in situ.

More generally, several embodiments of the invention can be used to deliver and express a desired ion channel, extracellular receptor, or intracellular signaling protein gene in selected cardiac tissues, particularly to modify the electrical properties of that tissue, e.g., increasing or decreasing the heart rate, increasing or decreasing its refractoriness, increasing or decreasing the speed of conduction, increasing or decreasing focal automaticity, and/or altering the spatial pattern of excitation. The general method involves delivery of genetic materials (DNA, RNA) by injection of the myocardium or perfusion through the vasculature (arteries, veins) or delivery by nearly any other material sufficient to facilitate transformation into the targeted portion of the myocardium using viral (adenovirus, AAV, retrovirus, other recombinant viruses) or non-viral vectors (plasmid, liposomes, protein-DNA combinations, lipid-DNA or lipid-virus combinations, other non-viral vectors) to treat cardiac arrhythmias.

By way of illustration, genes that could be used to affect cardiac firing rate include ion channels and pumps (α subunits or accessory subunits of the following: potassium channels, sodium channels, calcium channels, chloride channels, stretch-activated cation channels, HCN channels, sodium-calcium exchanger, sodium-hydrogen exchanger, sodium-potassium ATPase, sarcoplasmic reticular calcium ATPase), cellular receptors and intracellular signaling pathways (α or β-adrenergic receptors, cholinergic receptors, adenosine receptors, inhibitory G protein α subunits, stimulatory G protein α subunits, Gβγ subunits) or genes for proteins that affect the expression, processing or function processing of these proteins.

As discussed above, modified cells also may be administered to induce or modulate pacemaker activity of cells or a subject. Once source of modified cells are cardiac myocardial cells generated from differentiated (spontaneous or driven) stem cells, such as embryonic bone marrow cells. The stem-cell-derived cardiomyocytes exhibiting pacemaker function then may be implanted such as by catheter or injection to targeted cardiac tissue. Methods suitable for producing stem cell-derived cardiac myocytes are disclosed in e.g. U.S. Published Patent Application US20010024824A1 and U.S. Published Patent Application US20020022259A1, all herein incorporated by reference.

Similarly, existing cardiomyocytes may be transformed with a polynucleotide expression to provide desired pacemaker as discussed herein ex vivo and then implanted to targeted cardiac tissue of a subject e.g. by catheter or injection. Suitably, the existing cardiomyocytes may be harvested from the subject receiving treatment to facilitate delivery of those cells after modification (e.g., transformed with a polynucleotide expression system as disclosed herein) and re-administration.

The modified cells may have been harvested from the recipient, e.g., the subject to which the cells are administered. For example, bone marrow stem cells may be harvested from a subject and then differentiated to cardiomyocytes with pacemaker function. Cardiac cells, such as sino-atrial node cells may be harvested from a subject such as through removal via catheter or other protocol, modified e.g. by insertion of a desired polynucleotide delivery system as disclosed herein and then administered to the subject. As discussed above, as referred to herein, the administered cells preferably are modified in some respect prior to administration, such as differentiated stem cells or transformed with a polynucleotide expression system; modified administered cells as referred to herein would not include simply transplanted cardiac cells that had not been modified in some respect.

Preferred subjects for treatment in accordance with several embodiments of the invention include domesticated animals e.g., pigs, horses, dogs, cats, sheep, goats and the like; rodents such as rats, hamsters and mice; rabbits; and primates such as monkeys, chimpanzees etc. A highly preferred mammal is a human patient, preferably a patient who has need of or suspected of having need of cardiac rhythm disorder, such as those disclosed herein.

Preferred subject for treatment include those that are suffering from or susceptible to a disease or disorder as disclosed herein e.g. such as a cardiac-related syncope, particularly Stokes-Adam syncope; an abnormality of sinus node function such as persistent sinus bradycardia, sino-atrial (S-A) block manifested as S-A Wenckebach, complete S-A block or sinus arrest, and high-grade atriventricular block; or bradycardia-tachycardia syndrome or other bradycardia related condition.

The effective dose of the nucleic acid will be a function of the particular expressed protein, the particular cardiac arrhythmia to be targeted, the patient and his or her clinical condition, weight, age, sex, etc.

More specific advantages of several embodiments of the invention include ability to convey localized effects (by focal targeted gene delivery), reversible effects (by use of inducible vectors, including those already reported as well as new generations of such vectors, including but not limited to adeno-associated vectors using tetracycline-inducible promoters to express wild-type or mutant ion channel genes), gradedness (by use of inducible vectors as noted above, in which gradedness would be achieved by titration of the dosage of the inducing agent), specificity of therapy based on the identity of the gene construct, ability to regulate therapeutic action by endogenous mechanisms (nerves or hormones) based on the identity of the gene construct, and avoidance of implantable hardware including electronic pacemakers and AICDs, along with the associated expense and morbidity.

The following non-limiting examples are illustrative of several embodiments of the invention. All documents mentioned herein are incorporated herein by reference.

### Example 1

### Effect of Inhibition of Kir2 Channels on Latent Pacemaker Activity of Ventricilar Myocytes

### Materials and Methods

### Dominant-Negative Effects

Dominant-negative effects of Kir2.1AAA on I_{K1} expression were achieved using the approach of Herskowitz (See, for example I. Herskowitz, Nature 329, 219-22; (1987)). The GYG motif, three amino acids in the H5 region of potassium channels that play a key role in selectivity and pore function, were replaced with three alanines in Kir2.1.

### Vectors

A bicistronic adenoviral vector, encoding both enhanced green fluorescence protein (EGFP, Clontech, Palo Alto, Calif., USA) and Kir2.1AAA, was created using the adenovirus shuttle vectors pAdEGI (D. C. Johns, H. B. Nuss, E. Marban, J. Biol. Chem. 272, 31598-603. (1997) and pAdC-DBEcR (U. C. Hoppe, E. Marban, D. C. Johns, J. Clin. Invest. 105, 1077-84. (2000)) as previously described, all herein incorporated by reference. The full-length coding sequence of human Kir2.1 (kindly supplied by G. F. Tomaselli, Johns Hopkins University) was cloned into the multiple cloning site of pAdEGI to generate pAdEGI-Kir2.1. The dominant-negative mutation GYG.fwdarw.AAA was introduced into Kir2.1 by site-directed mutagenesis, creating the vector pAdEGI-Kir2.1AAA. Adenovirus vectors were generated by Cre-lox recombination of purified 5 viral DNA and shuttle vector DNA as described (D. C. Johns, R. Marx, R. E. Mains, B. O'Rourke, E. Marban, J. Neurosci. 19, 1691-7. (1999)), herein incorporated by reference. The recombinant products were plaque purified, expanded, and purified on CsCl gradients yielding concentrations on the order of 10¹⁰ plaque-forming units (PFU) per milliliter.

### In Vivo Gene Delivery

Intracardiac injection was achieved by injection into the left ventricularcavity of adult guinea pigs (250-300 g) following lateral thoracotomy. The aorta and pulmonary artery were first cross-clamped and then a 30 gauge needle was inserted at the apex, enabling injection of the adenovirus solution into the left ventricular chamber. A total volume of 220 μl of adenovirus mixture was injected, containing 3×10¹⁰ PFU AdC-DBEcR and 2×10¹⁰ PFU AdEGI (control group) or 3×10¹⁰ PFU AdC-DBEcR and 3×10¹⁰ PFU AdEGI-Kir2.1AAA (knock-out group). The aorta and pulmonary artery remain occluded for 40-60 seconds before the clamp is released. This procedure allows the virus to circulate down the coronaries while the heart is pumping against a closed system and results in a widespread distribution of transduced cells. After the chest was closed, animals were injected intraperitoneally with 40 mg of the nonsteroidal ecdysone receptor agonist, GS-E ([N-(3-methoxy-2-ethylbenzoyl)N′-(3,5-dimethylbenzoyl)N′-tertbutylhydrazine]; kindly provided by Rohm and Haas Co., Spring House, Pa., USA), dissolved in 90 μl DMSO and 360 μl sesame oil.

### Transduction Efficiency

Transduction efficacy was assessed by histological evaluation of microscopic sections 48 hours after injection of AdCMV-βgal (160 μl of 2×10¹⁰ pfu/ml) into the LV cavity. After the animals were killed, hearts were excised, rinsed thoroughly in PBS, and cut into transverse sections. The sections were fixed in 2% formaldehyde/0.2% glutaraldehyde and stained in PBS containing 1.0 mg/ml 5-bromo-4-chloro-3-indolyl-D-galactoside (X-gal) as previously described (J. K. Donahue et al., Nat Med 6, 1395-8. (2000)). The sections were embedded in paraffin, cut to 5 μm thickness and stained with X-gal solution for visual assessment oftransduction efficacy (J. K. Donahue et al., Nat. Med. 6, 1395-8. (2000), herein incorporated by reference). This gene delivery method achieved transduction of approximately 20% of ventricular myocytes throughout the LV wall.

Sixty to 72 hours after injection, guinea-pig left ventricular myocytes were isolated using Langendorffperfusion and collagenase digestion (R. Mitra, M. Morad, Proc Natl Acad Sci USA 83, 53404. (1986); U. C. Hoppe, D. C. Johns, E. Marban, B. O'Rourke, Circ Res 84, 964-72. (1999), herein incorporated by reference. Dissociationstypically yielded 60-70% viable myocytes. A xenon arc lamp was used to view GFP fluorescence at 488/530 nm (excitation/emission). Transduced myocytes were identified by their green fluorescence using epifluorescence. The yield of transduced and viable isolated myocytes using the LV cavity injection approach (−20%) was much higher than with direct intramyocardial injection (U. C. Hoppe, E. Marban, D. C. Johns, J Clin Invest 105, 1077-84. (2000); U. C. Hoppe, E. Marban, D. C. Johns, Proc Natl Acad Sci USA 98, 5335-40. (2001), all herein incorporated by reference).

Cellular recordings were performed using the whole-cell patch clamp technique (24) with an Axopatch 200B amplifier (Axon Instruments, Foster City, Calif., USA) while sampling at 10 kHz (for currents) or 2 kiHz (for voltage recordings) and filtering at 2 kHz. Pipettes had tip resistances of 24 M.OMEGA. when filled with the internal recording solution. Cells were superfused with a physiological saline solution containing (in mM) 140 NaCl, 5 KCl, 2 CaCl₂, 10 glucose, 1 MgCl₂, 10 HEPES; pH was adjusted to 7.4 with NaOH. For I_{K1} recordings, CaCl₂ was reduced to 100 pM, CdCl₂ (200 μM) was added to block I_{caL}, and I_{Na} was steady-state inactivated by using a holding potential of −40 mV. To obtain I_{K1} as a barium (Ba²⁺)-sensitive current, background currents remaining after the addition of Ba²⁺ (500 μM) were subtracted from the records. The pipette solution was composed of (in mM) 130 K-glutamate, 19 KCl, 10 Na-Hepes, 2 EGTA, 5 Mg-ATP, 1 MgCl₂; pH was adjusted to 7.2 with KOH. Data were not corrected for the measured liquid junction potential of −12 mV. Action potentials were initiated by briefdepolarizing current pulses (2 ms, 500-800 pA, 110% threshold) at 0.33 Hz. Action potential duration (APD) was measured as the time from the overshoot to 50% or 90% repolarization (APD₅₀, APD₉₀, respectively). For I_{Ca,L} recordings, cells were superfused with a saline solution containing (in mM) 140 N-methyl-D-glucanine, 5 CsCl, 2 CaCl₂, 10 glucose, 0.5 MgCl₂, 10 HEPES; pH was adjusted to 7.4 with HCl The pipette solution was composed of (in MM) 125 CsCl, 20 TEA-Cl, 2 EGTA, 4 Mg-ATP, 10 HEPES; pH was adjusted to 7.3 with CsOH. Data reported are means.+−.S.E.M. with P<0.05 (t test) indicating statistical significance.

All recordings were performed at physiologic temperature (37.degree. C.) and 60-72 hours after in vivo transduction. Given that adenovirus infection itself does not modify the electrophysiology of guinea-pig myocytes (U. C. Hoppe, E. Marban, D. C. Johns, J Clin Invest 105, 1077-84. (2000)), patch-clamp experiments performed on nontransduced (non-green) left ventricular myocytes isolated from AdEGI-Kir2.1AAA-injected animals (APD₅₀=233.8.+−0.10.5 ms, n=6), as well as on green cells from AdEGI-injected hearts (APDso=247.6.+−0.10.3 ms, n=24, P=0.52), were used as controls.

### Electro Cardiographs

Surface ECGs were recorded immediately after operation and 72 hours after intramyocardial injection as previously described (U. C. Hoppe, E. Marban, D. C. Johns, J Clin Invest 105, 1077-84. (2000); U. C. Hoppe, E. Marban, D. C. Johns, Proc Nail Acad Sci USA 98, 533540. (2001)). Guinea pigs were sedated with isoflurane, and needle electrodes were placed under the skin. Electrode positions were optimized to obtain maximal amplitude recordings, enabling accurate measurements of QT intervals. ECGs were simultaneously recorded from standard lead 11, and modified leads I and III. The positions of the needle electrodes were marked on the guinea pigs' skin after recording, to ensure exactly the same localization 72 hours later. The rate corrected-QT interval (QTc) was calculated (E. Hayes, M. K. Pugsley, W. P. Penz, G. Adaikan, M. J. Walker, J Pharmacol. Toxicol. Methods 32, 201-7. (1994)).

Results Replacement of three critical residues in the pore region of Kir2.1 by alanines (GYGI₁₄₄₋₁₄₆-AAA, or Kir2.1AAA) creates a dominant-negative construct which suppresses current flux when co-expressed with wild-type Kir2.1. In oocytes, injection of Kir2.1 AAA RNA alone does not produce current, but coinjection with wild-type Kir2.1 RNA causes suppression of I_{K1}. Incorporation of a single mutant subunit within the tetrameric Kir channel is sufficient to knock out function. The dominant-negative effects are specific to the Kir family of potassium channels, as Kv1.2 currents were not reduced by co-injection with Kir2.1AAA RNA. When Kir2.1AAA was transiently expressed in a mammalian cell line (HEK) stably expressing Kir2.1, the dominant-negative construct reduced I_{K1} by approximately 70%.

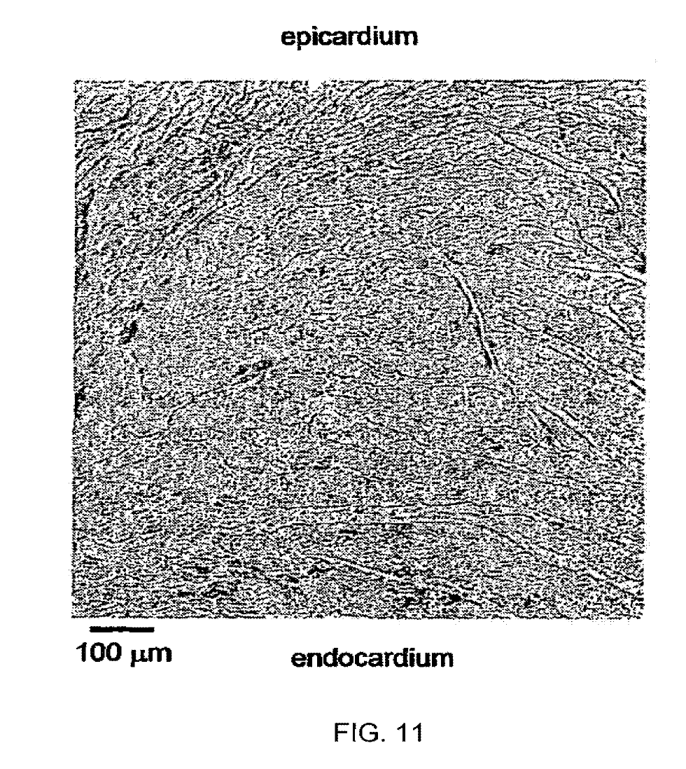

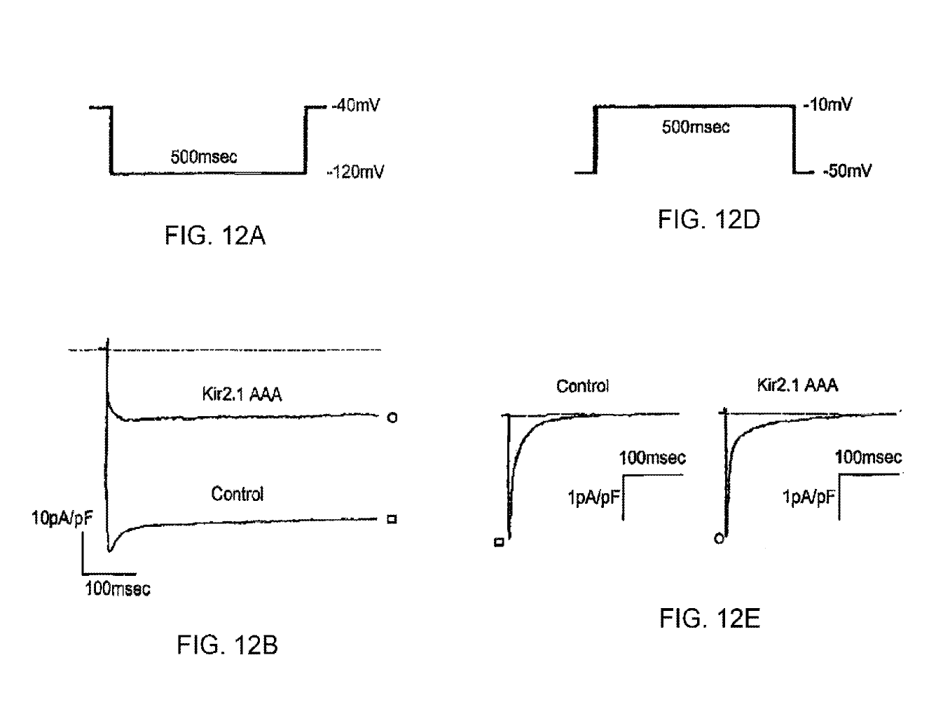

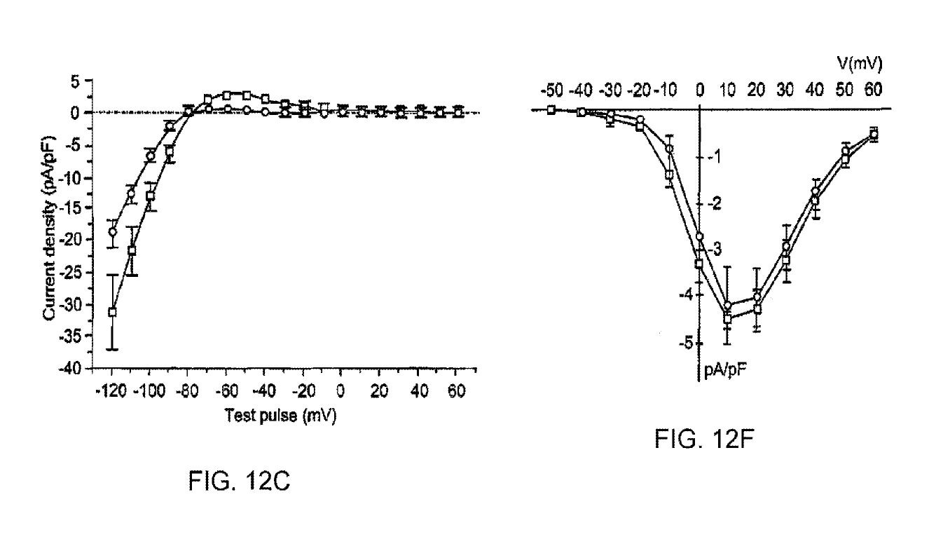

Kir2.1AAA was packaged into a bicistronic adenoviral vector and injected into the left ventricular cavity of guinea pigs. This method of delivery sufficed to achieve transduction of .about.20% of ventricular myocytes (FIG. 11). Myocytes isolated 34 days after in vivo transduction with Kir2.1AAA exhibited suppression Of I_{K1} (FIG. 12B,C), but calcium currents remained unchanged (FIG. 12E,F).

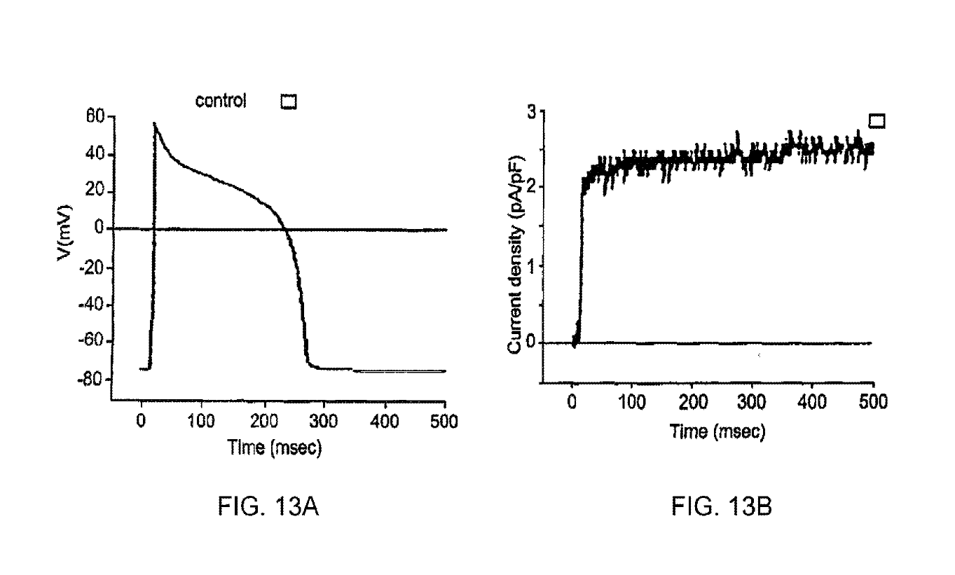

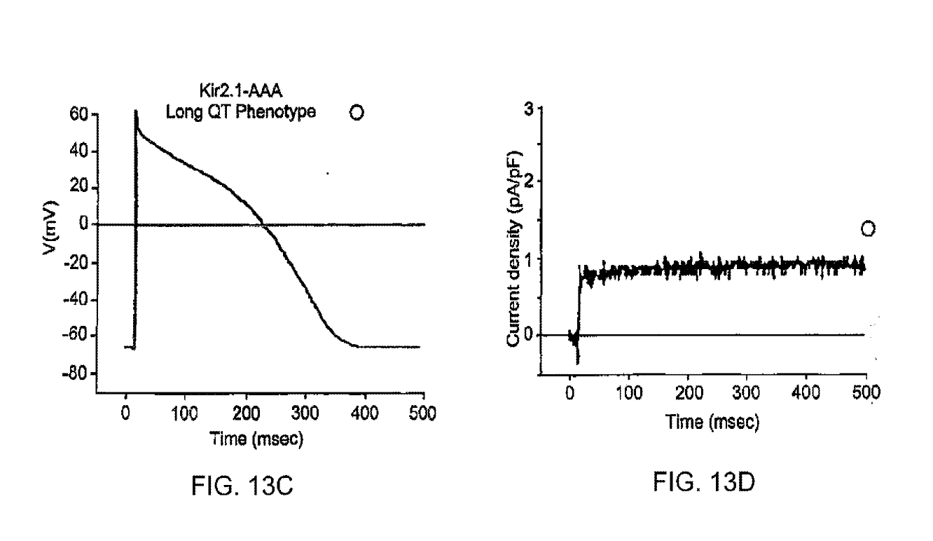

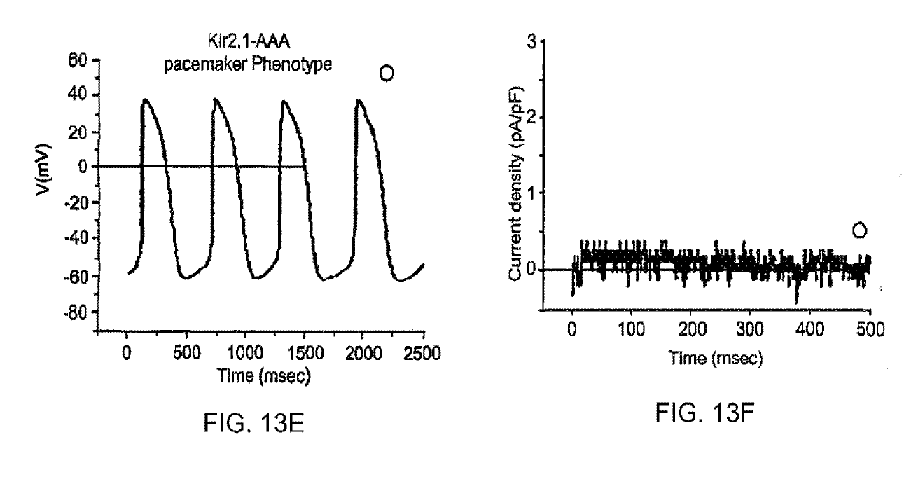

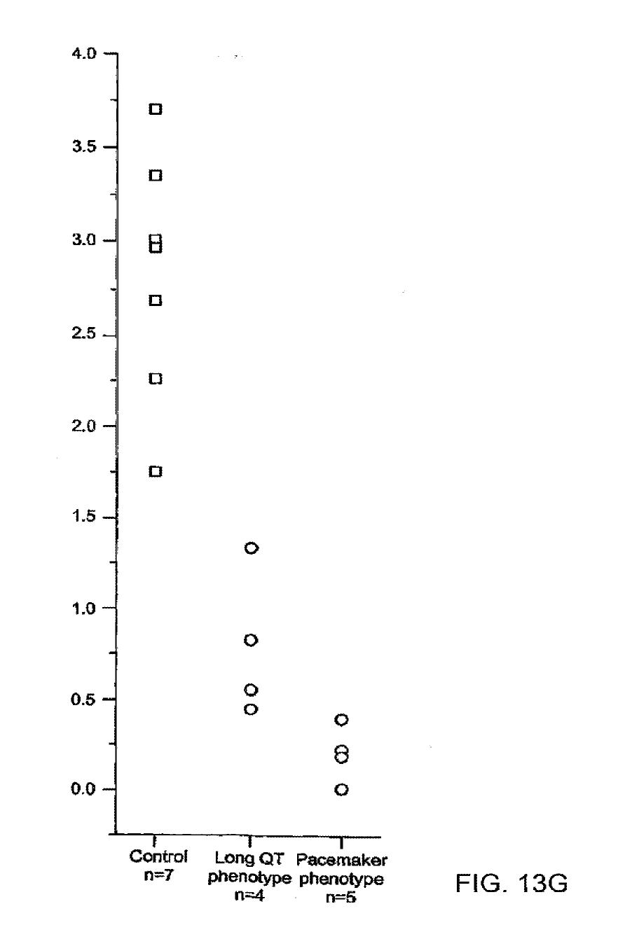

Control ventricular myocytes exhibited no spontaneous activity, but did fire single action potentials when subjected to depolarizing external stimuli (FIG. 13A). In contrast, Kir2.1AAA myocytes exhibited either of two phenotypes: a stable resting potential from which prolonged action potentials could be elicited by external stimuli (FIG. 13C, “long QT phenotype”), or spontaneous activity (FIG. 13E). Prolongation of action potentials would be expected to lengthen the QT interval of the electrocardiogram (long QT phenotype), whereas the spontaneous activity resembles that of genuine pacemaker cells; the maximum diastolic potential is relatively depolarized, with repetitive, regular and incessant electrical activity initiated by gradual “phase 4” depolarization and a slow upstroke (Table 1). The different phenotypes correspond to three distinct ranges of I_{K1} density (FIG. 12B,D,F,G). Thus, Kir2.1AAA-transduced myocytes exhibit either a long QT phenotype or a pacemaker phenotype, depending upon how much suppression of I_{K1} happened to have been achieved in that particular cell. Myocytes in which I_{K1} was suppressed below 0.4 pA/pF (at −50 mV) all exhibited spontaneous AP, while myocytes with greater than 0.4 pA/pF I_{K1} had stable resting membrane potentials and prolonged action potentials.

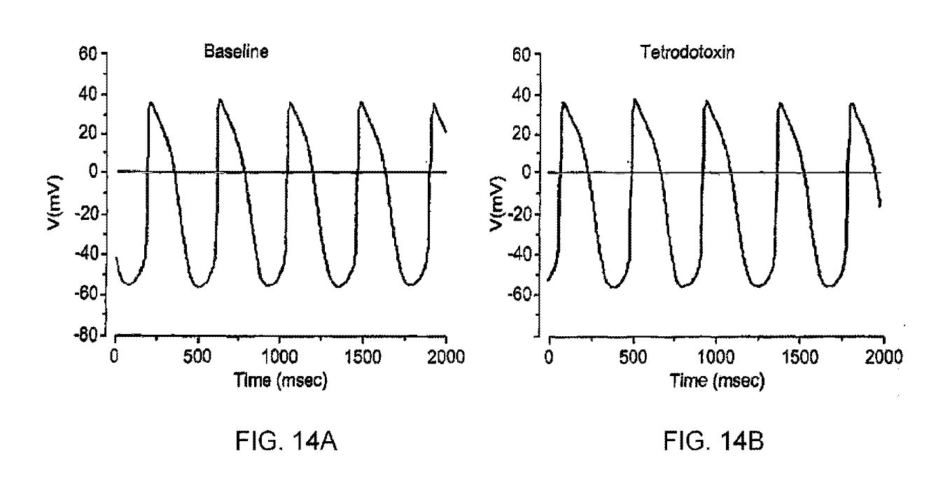

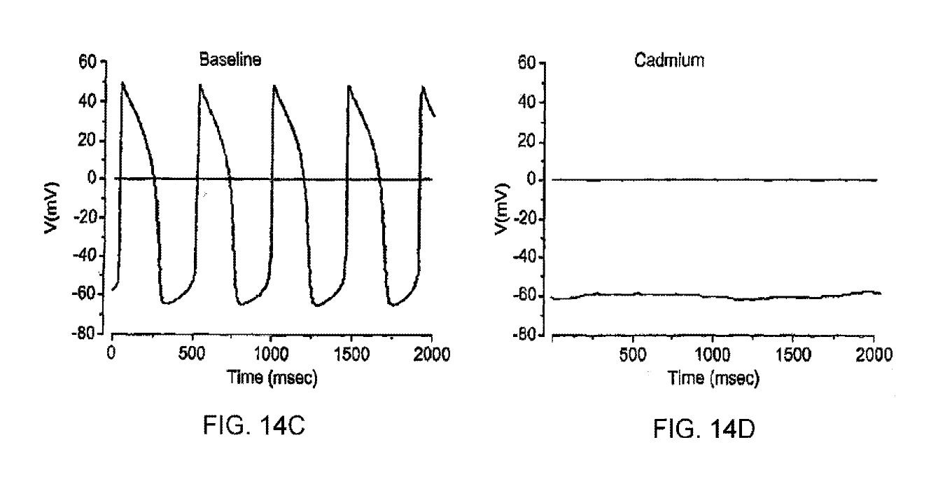

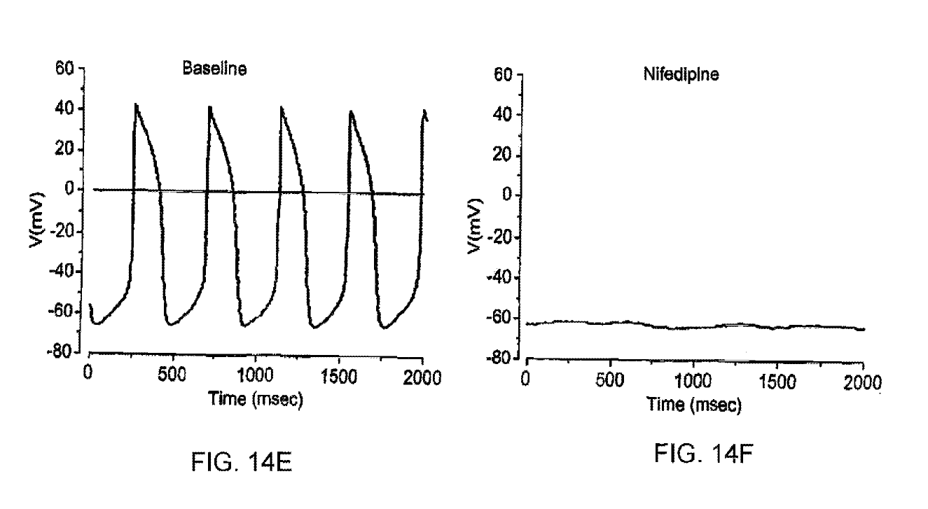

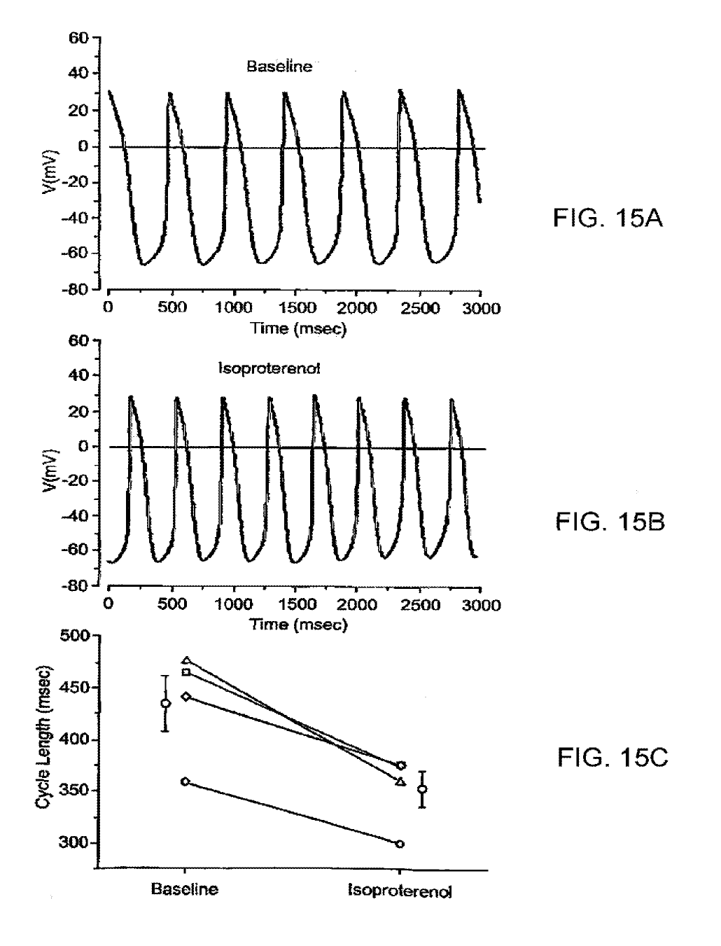

Cells with a pacemaker phenotype were unaffected by the Na channel blocker tetrodotoxin (FIG. 14A,B), but spontaneous firing ceased during exposure to calcium channel blockers (cadmium, FIG. 14C,D; nifedipine, E,F). Thus, the excitatory current underlying spontaneous action potentials is carried by calcium channels, as is the case with genuine pacemaker cells. Likewise, Kir2.1AAA spontaneous-phenotype cells responded to beta-adrenergic stimulation just as nodal cells do, increasing their pacing rate (FIG. 15) to accelerate the heart rate.

Table 2. Action potential characteristics in control, long QT phenotype Kir2.1AAA, and pacemaker phenotype Kir2.1AAA myocytes. In all of the control (n=30) and long QT phenotype Kir2.1AAA cells (7 of 22 Kir2.1AAA cells), stable action potentials were evoked in response to electrical stimulation. The pacemaker phenotype Kir2.1AAA cells (15 of 22 Kir2.1AAA cells) exhibited the spontaneous action potentials with no input stimulus.

| | Maximum | Spontaneous | Maximum | | |
|---|---|---|---|---|---|
| | diastolic | action potential | upstroke | | |
| Cells | potential (mV) | rate (APs/min) | velocity (V/s) | APD₅₀ (ms) | APD₉₀ (ms) |
| Control | −75.3 ± 0.7 | N/A | 101.3 ± 3.3 | 244.8 ± 8.5 | 271.1 ± 8.5 |
| Long QT | −68.0 ± 23* | N/A | 92.4 ± 7.0 | 271.9 ± 19.5 | 353.4 ± 17.4* |
| phenotype | | | | | |
| Kir2.1AAA | | | | | |
| Pacemaker | −60.7 ± 2.1* | 116.8 ± 10.9* | 15.2 ± 4.5* | 232.8 ± 20.3* | |
| phenotype | | | | | |
| Kir2.1AAA | | | | | |

*P < 0.05 Kir2.1AAA vs. control
APD₅₀ and APD₉₀ are measurements of action potential duration taken from the AP overshoot to 50% or 90% repolarization (APD₅₀, APD₉₀, respectively).

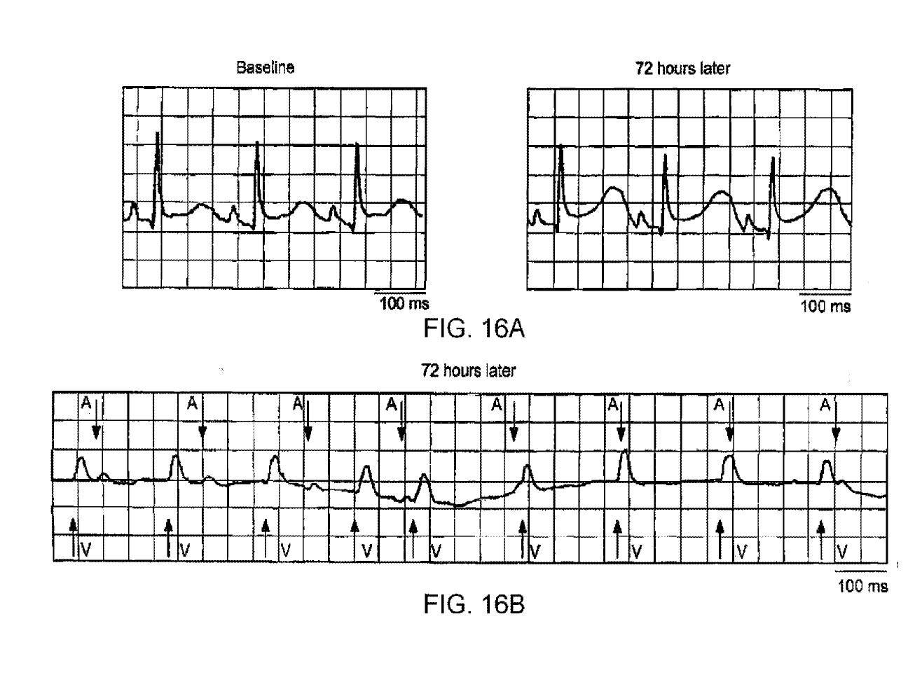

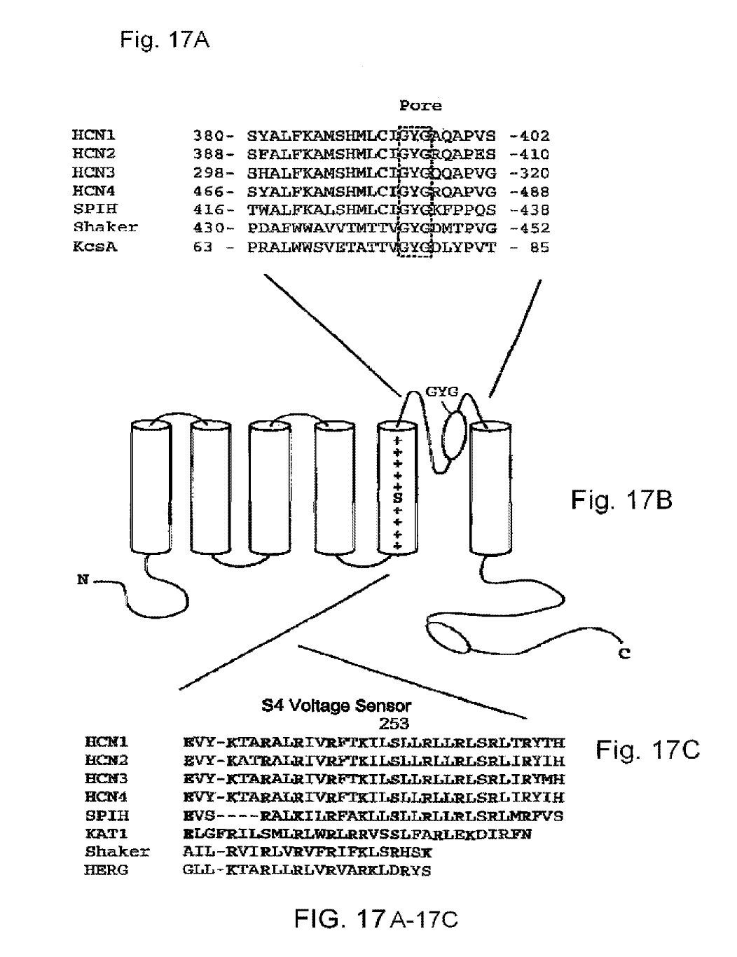

Electrocardiography revealed two phenotypes. FIG. 16A shows a prolongation of the QT interval (FIG. 16A). Nevertheless, 40% of the animals exhibited an altered cardiac rhythm indicative of spontaneous ventricular foci (FIG. 16B). Premature beats of ventricular origin can be distinguished by their broad amplitude, and can be seen to “march through” to a beat independent of that of the physiological sinus pacemaker. In normal sinus rhythm, every P wave is succeeded by a QRS complex. However, if ectopic beats arise from foci of induced pacemakers, the entire heart can be paced from the ventricle. Indeed, ventricular automaticity developed in two of five animals 72 hours after transduction with Kir2.1AAA. In these two animals, P waves were not followed by QRS complexes; both P waves and QRS complexes maintained independent rhythms. The RR intervals were shorter than the PP intervals, signifying a rhythm of ventricular origin (accelerated ventricular rhythm due to automaticity). The two phenotypes in vivo correspond well to the distinct long QT and pacemaker cellular phenotypes.

The dominant negative results demonstrate the durability and regional specificity of the methods used herein. These results demonstrate that the specific suppression of Kir2 channels suffices to unleash pacemaker activity in ventricular myocytes. These results also demonstrate that the important factor for pacing is solely the absence of the strongly-polarizing I_{K1}, rather than the presence of special genes (although such genes may play an important modulatory role in genuine pacemaker cells).

### Example 2

### The Triple Niutation GYG₃₆₅₋₃₆₇AAA Rendered HCN1 Channels Non Functional

### Molecular Biology and Heterologous Expression

mHCN1 and mHCN2 were subcloned into the pGH expression vector. B. Santoro et al., Cell, 93:717-29 (1998). Site-directed mutagenesis was performed using polymerase chain reaction (PCR) with overlapping mutagenic primers. All constructs were sequenced to ensure that the desired mutations were present. cRNA was transcribed from NheI- and SphI-linearized DNA using T7 RNA polymerase (Promega, Madison, Wis.) for HCN1 and HCN2 channels, respectively. Channel constructs were heterologously expressed and studied in Xenopus oocytes. Briefly, stage 1V through VI oocytes were surgically removed from female frogs anesthetized by immersion in 1% tricaine (3-aminobenzoic acid ethyl ester) followed by digestion with 2 mg/mL collagenase in OR-2 containing (in mM): 88 NaCl, 2 KCl, I MgCl2 and 5 mM HEPES (pH 7.6 with NaOH) for 30 to 60 minutes. Isolated oocytes were injected with cRNA (1 ng/nL) as indicated, and stored in ND96 solution containing (in mM) 96 NaCl), 2 KCl, 1.8 CaCl₂, 1 MgCl₂ and 5 HEPES (pH 7.6) supplemented with 50 μg/mL gentamicin, 5 mM pyruvate and 0.5 mM theophylline for 1-4 days before experiments. It was found that injection with 50-100 ng of total cRNA per cell was sufficient to attain maximal expression while 10-25 ng/cell corresponds to the range linearly proportional to the expressed current amplitude.

### Electrophysiology

Two-electrode voltage-clamp recordings were performed at room temperature (23-25.degree. C.) using a Warner OC-725C amplifier (Hamden, Conn.). Agarose-plugged electrodes (TW120E-6; World Precision Instruments) were pulled using a Sutter P-87 horizontal puller, filled with 3 M KCl and had final-tip resistances of 24 Ma The recording bath solution contained (in mM): 96 KCl, 2 NaCl, 10 HEPES, and 2 MgCl₂ (pH 7.5 with KOH). The currents were digitized at 10 kHz and low-pass filtered at 1-2 kHz (−3 dB). Acquisition and analysis of current records were performed using custom-written softwares.

### Experimental Protocols and Data Analysis

The steady-state current-voltage (I-V) relationship was determined by plotting the HCN1 currents measured at the end of a 3-second pulse ranging from −150 to 0 mV at 10 mV increments from a holding potential of −30 mV. The voltage dependence of HCN channel activation was assessed by plotting tail currents measured immediately after pulsing to .about.140 mV as a function of the preceding 3-second test pulse normalized to the maximum tail current recorded. Data were fit to the Boltzmann functions using the Marquardt-Levenberg algorithm in a non-linear least-squares procedure:

m=1{+exp[(Vₜ−V_{1/2})/k]}

where Vₜ is the test potential, V_{1/2} is the half-point of the relationship and k=RT/zF is the slope factor.

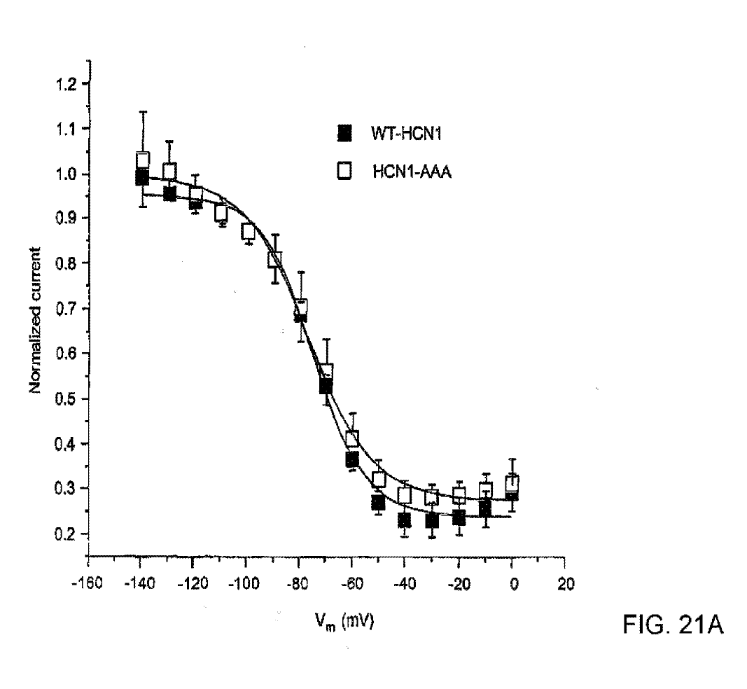

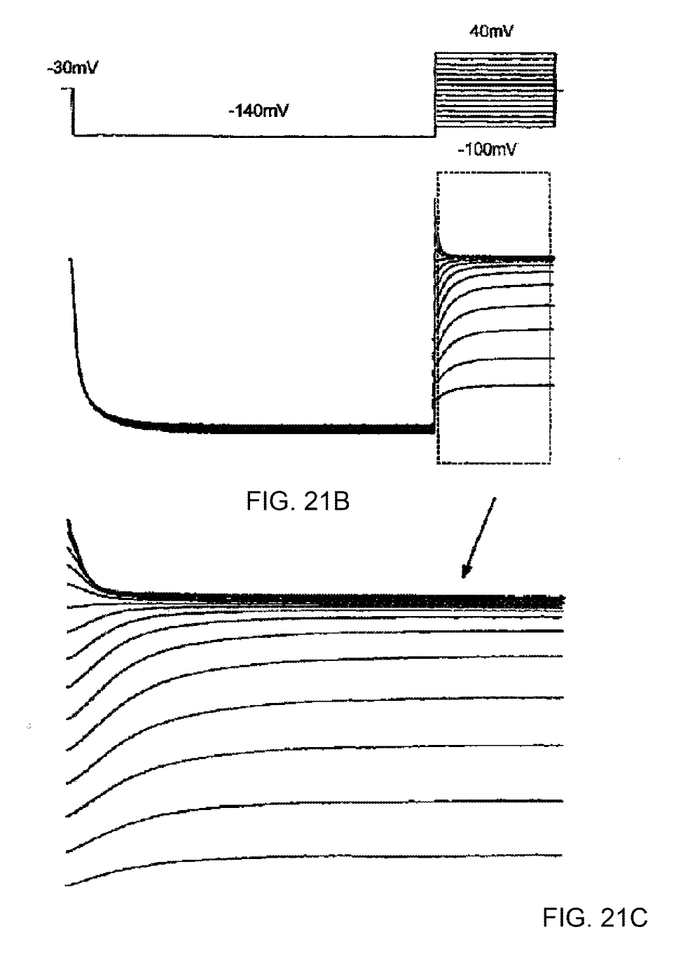

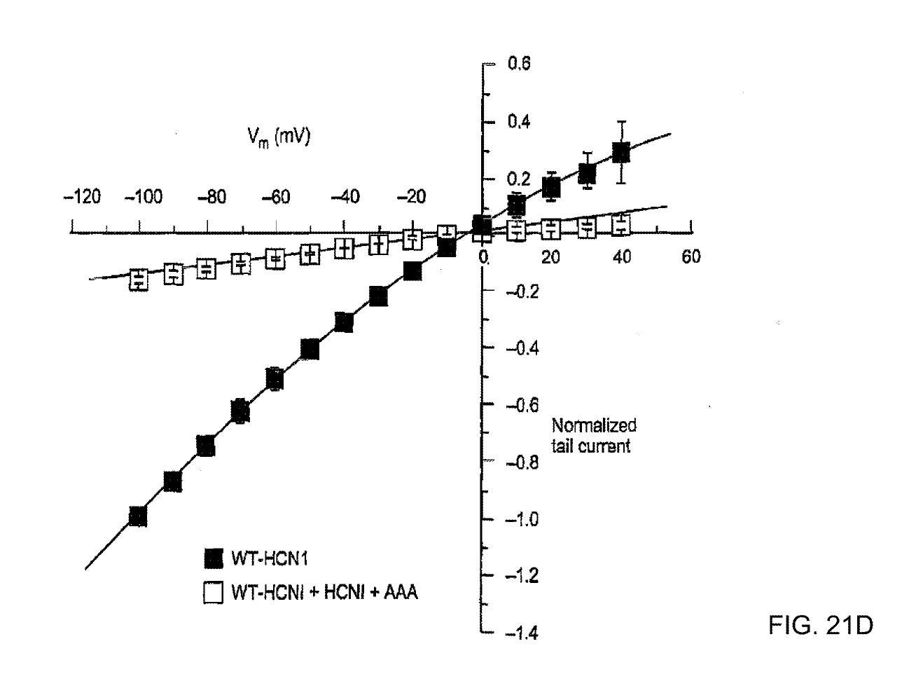

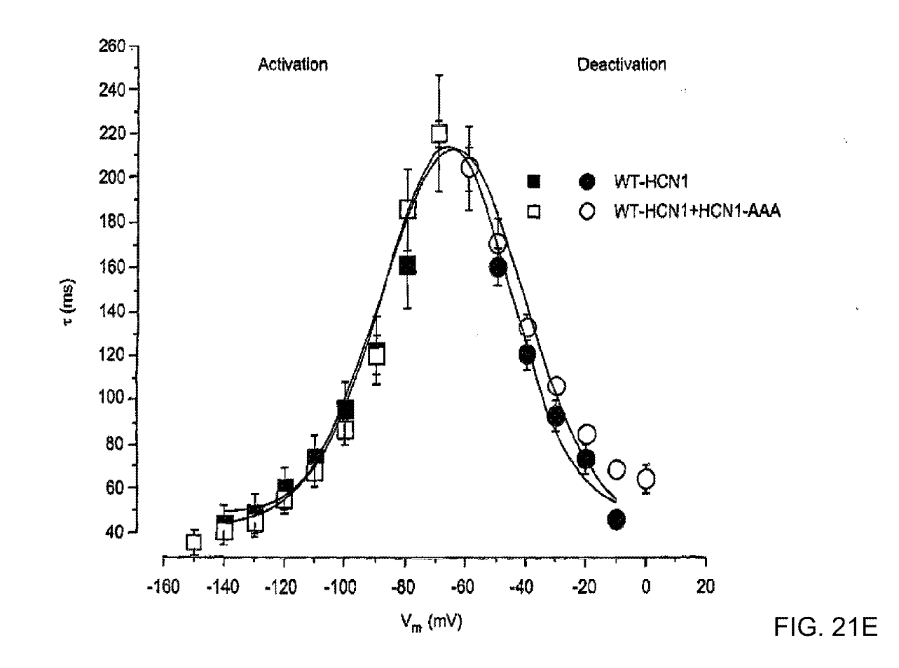

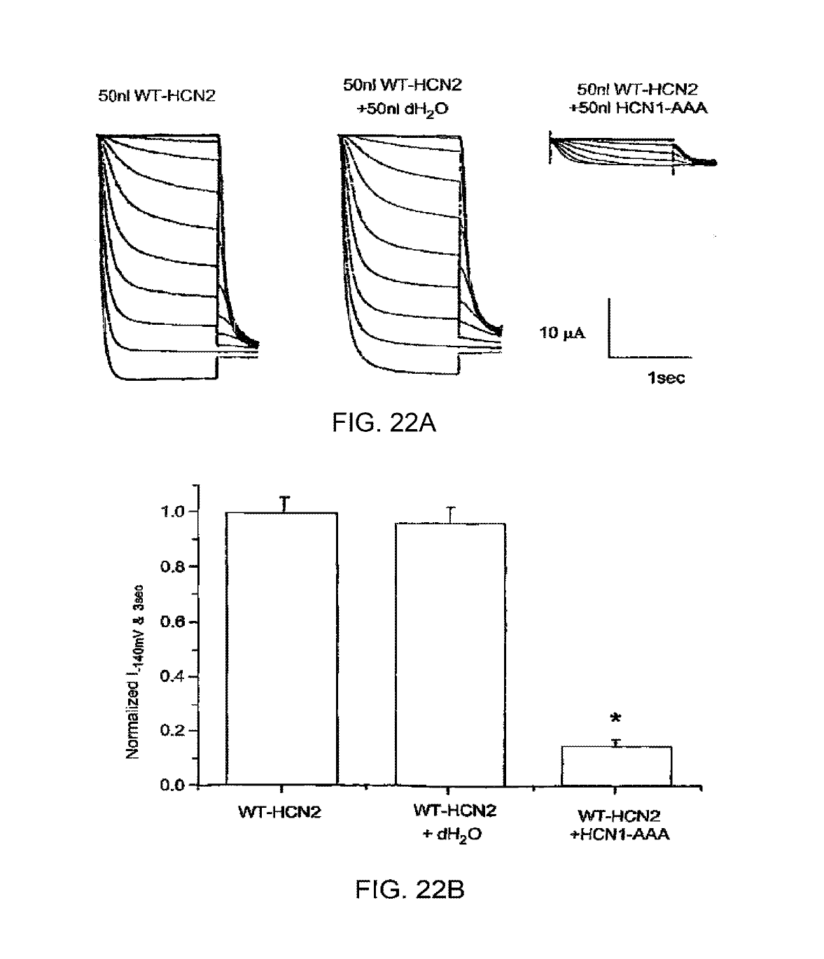

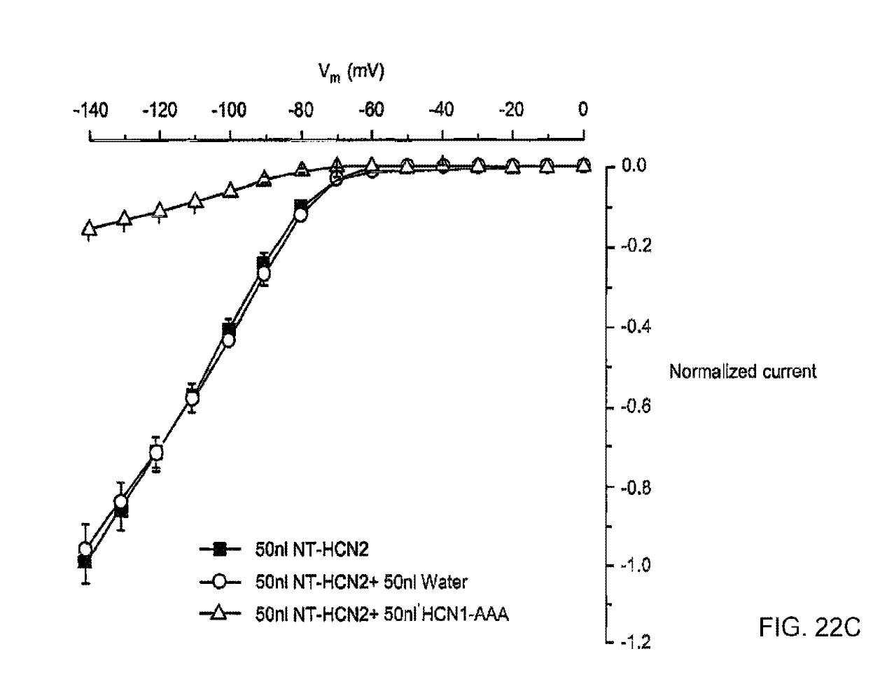

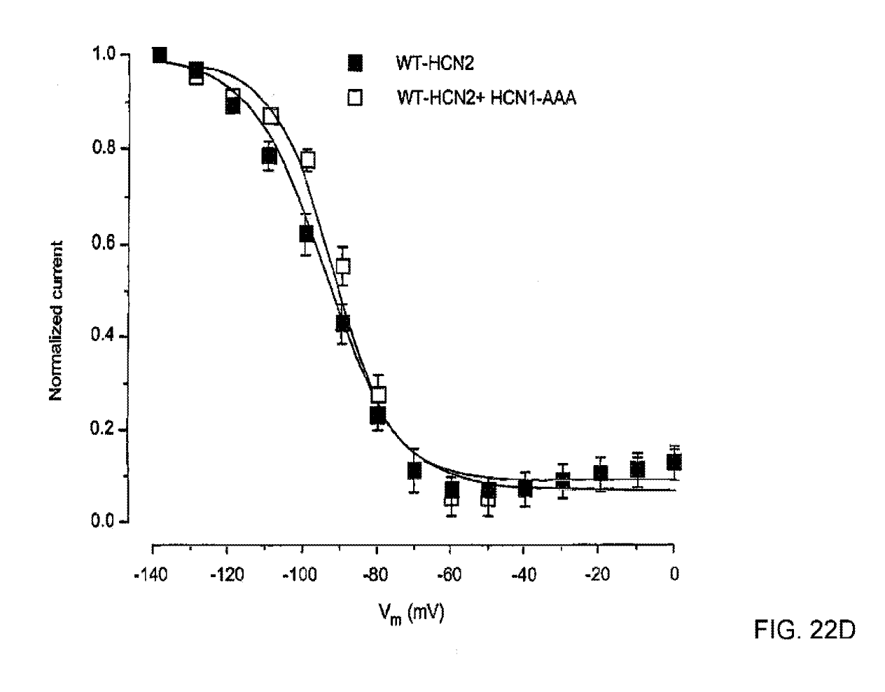

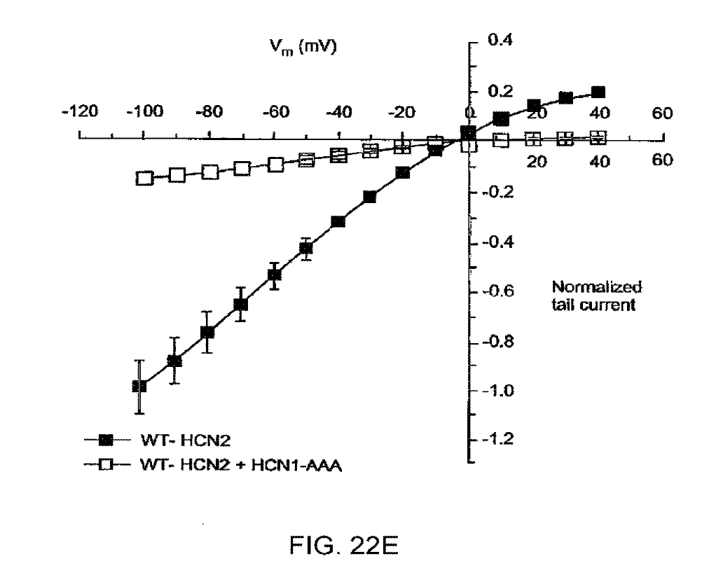

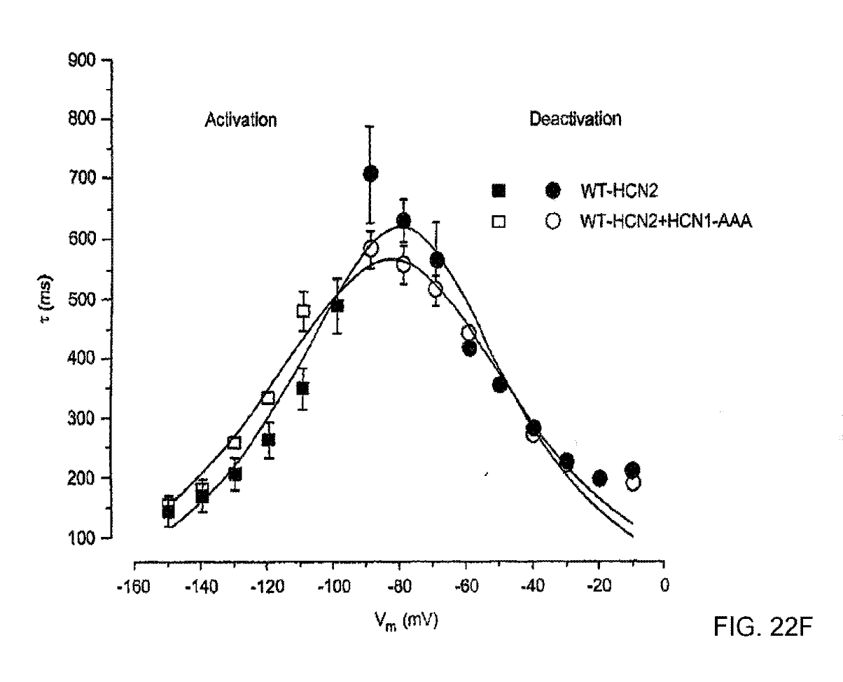

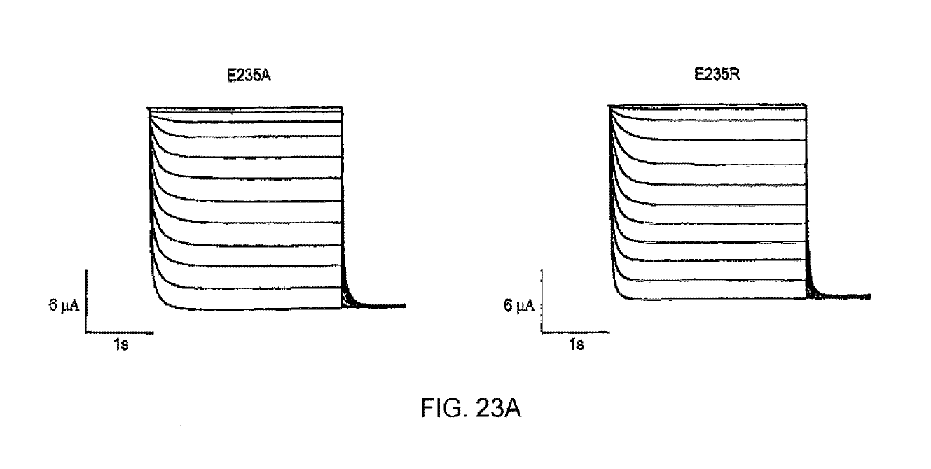

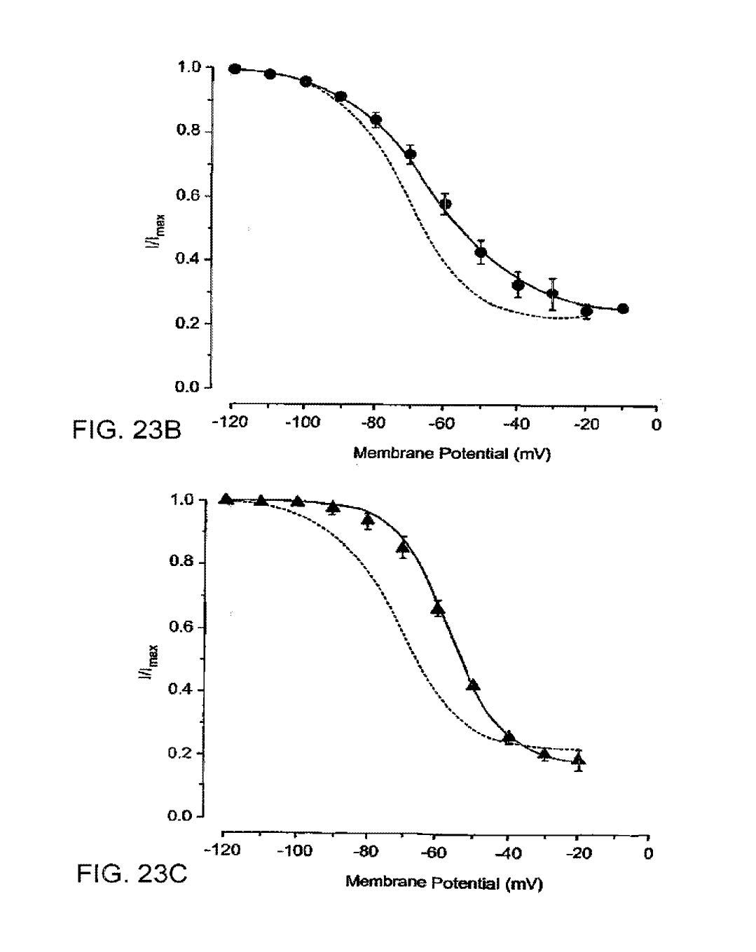

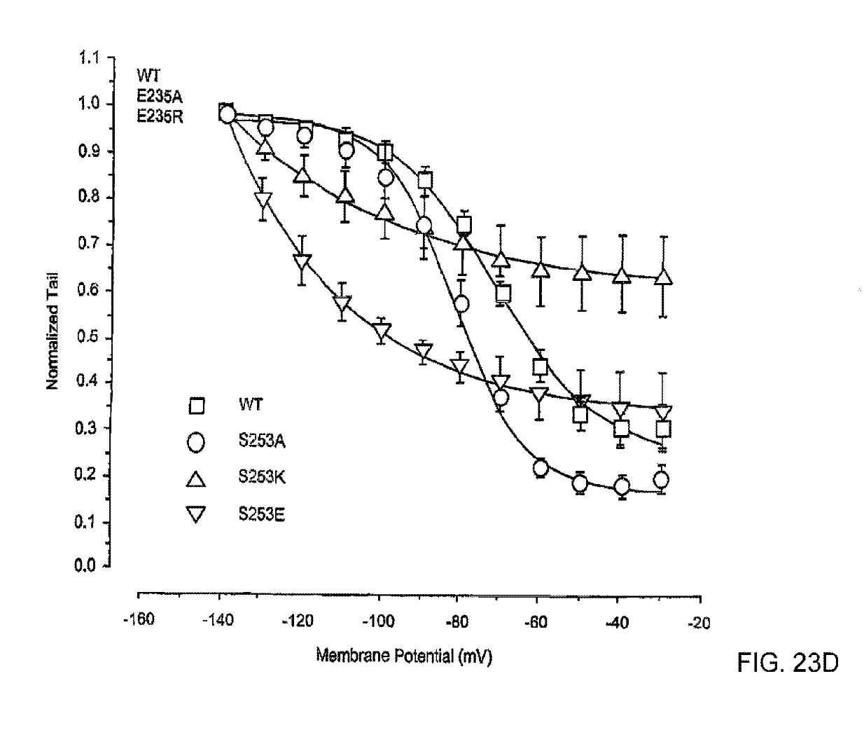

For reversal potentials (Eᵣₑᵥ), tail currents were recorded immediately after stepping to a family of test voltages ranging from −100 to +40 mV preceded by a 3-s prepulse to either −140 (cf. FIG. 21B) or −20 mV. The difference oftail currents resulting from the two prepulse potentials was plotted against the test potentials, and fitted with linear regression to obtain Eᵣₑᵥ. For current kinetics, the time constants for activation (τact) and deactivation (τdeact) were estimated by fitting macroscopic and tail currents, respectively, with a mono-exponential function.

Data are presented as mean.+−.SEM. Statistical significance was determined using an unpaired Student's t-test with p<0.05 representing significance.

The effects of the triple HCN1 mutation GYG₃₆₅₋₃₆₇AAA (HCN1-AAA) on channel function were evaluated by individually expressing WT and mutant channel constructs. FIG. 18 shows that hyperpolarization of oocytes injected with WT HCN1 cRNA to potentials below −40 mV elicited time-dependent inward currents that reached steady state current amplitudes after .about.500 ms. Currents increased in amplitude with progressive hyperpolarization. In contrast, uninjected oocytes and those injected with HCN1-AAA did not yield measurable currents, indicating that the triple alanine substitution rendered HCN1 completely non-functional.

### HCN1 AAA Suppressed the Normal Activity of WTHCN1 in a Dominant-Negative Manner

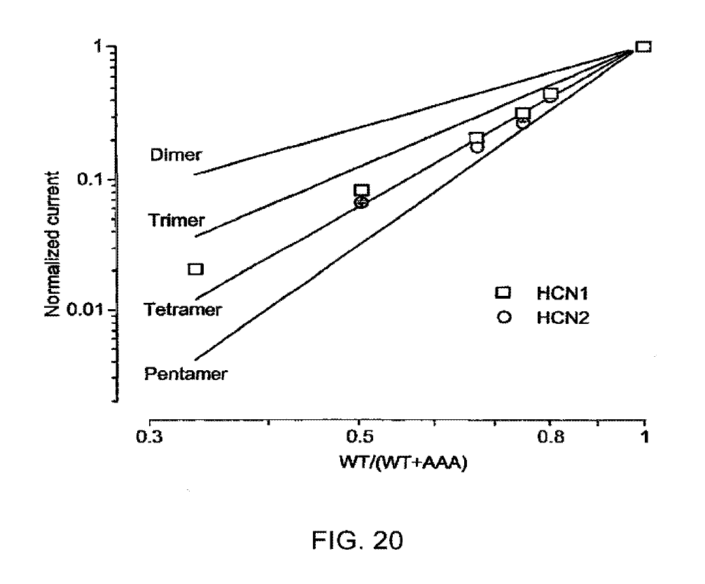

Previous studies have identified numerous ion channel mutations that are capable of crippling channel activities in a dominant-negative manner when normal and defective subunits coassemble to form multimeric complexes. R. Li et al., J Physiol, 533: 127-33 (2001); J. Seharaseyon, J. Mol. Cell. CardioL, 32:1923-30 (2000); MT Perez-Garcia, J Neurosci, 20:5689-95 (2000); J. Seharseyon et al., J Biol. Chem., 275: 17561-5 (2000); UC Hoppe et al., J Clin Invest, 105:1077-84 (2000); M J Lalli et al., Pflugers Arch, 436:957-61 (1998); D C John et al., J Biol Chem, 272:31598-603 (1997), all herein incorporated by reference. HCN1-AAA was anticipated to exert a dominant-negative effect when combined with WT HCN1 subunits. That was tested by co-expressing both WT HCN1 and HCN1-AAA channel constructs. FIG. 19 shows that oocytes co-injected with 50 nL WT HCN1 and 50 nL HCN1-AAA cRNA (concentration=1 ng/nL) expressed currents 85.2.+−.1.9% (n=8) smaller than cells injected with 50 nL of WT HCN1 cRNA alone when measured at −140 mV after the same incubation period (p<0.01; FIG. 19B). Such quantitative differences existed throughout almost the entire activation range of HCN1 channels as indicated by their corresponding steady-state current-voltage relationships (FIG. 19C). These observations demonstrate that HCN1-AAA could suppress the normal activity of WT HCN1 channels in a dominant-negative fashion despite the presence of the same numbers of functional subunits (assuming equal RNA stability and translation efficiencies, as is conventional in previous K⁺ channel studies, Hille B. Ion channels of Excitable Membranes. 3rd Edition. Sunderland, Mass., U.S.A. Sinauer Associates, Inc. 2001; R. MacKinnon Nature. 1991; 350:232-5.). In contrast, co-injection of 50 nL WT HCN1 cRNA with an equal volume of dH₂O yielded current magnitudes not different from the injection of 50 nL WT HCN1 alone (p>0.05), suggesting that the dominant-negative suppressive effects observed with HCN 1-AAA were not due to non-specific mechanisms such as mechanosensitive effects. We also studied the effects of varying the ratio of WT HCN 1:HCN 1-AAA and WT HCN2:HCN 1-AAA while maintaining the total cRNA injected constant (25 ng was used to prevent saturation of expression). As anticipated from a dominant-negative mechanism, current suppression increased as the proportion of HCN1-AAA increased (55.2.+−.3.2, 68.3.+−.4.3, 78.7.+−.1.6, 91.7.+−.0.8, 97.9.+−.0.2% current reduction for WTHCN1:HCN1-AAA ratios of 4:1, 3:1, 2:1, 1:1 and 1:2, respectively; FIG. 20).

Co-Expression of the Dominant-Negative Construct HCN1-AAA with WT HCN1 Did Not Alter Normal Gating and Permeation Properties

The affect of co-expression of HCN1-AAA with WT HCN1 on gating and permeation properties in addition to its dominant-negative suppressive effects on current amplitudes were then investigated. FIG. 21A shows that both the midpoints and slope factors derived from the steady-state activation curves of WT HCN1 alone (V_{1/2}=−76.7.+−.0.8 mV; k=13.3.+−.0.6 mV; n=15) and after suppression by HCN1-AAA (ratio=1:1; V_{1/2}=−77.0.+−.1.7 mV; k=12.3.+−.1.0 mV; n=12) were identical (p>0.05). Tail current-voltage relationships also indicate that whereas whole-cell currents were suppressed by HCN1-AAA, the reversal potential was not changed (WT HCN1 alone=−4.5.+−.1.4 mV, n=8; WT+AAA=−5.25.+−.0.8 mV, n=5; p>0.05; FIG. 21B&C). Similarly, the time constants for current activation (τdeact) and deactivation (τdeact), whose distribution was bell-shaped with midpoints comparable to those derived from the corresponding steady-state activation curves, were also unaltered after HCN1-AAA suppression across the entire voltage range studied (p>0.05; FIG. 21D). Taken together, our observations indicate that the non-suppressed currents exhibited normal gating and permeation phenotypes.

### HCN1 AAA Suppressed WT HCN2 Currents Without Altering Gating and Permeation.

If different HCN isoforms can coassemble to form heteromeric channel complexes, HCN1-AAA should also suppress the activities of WT HCN2 channels in a dominant-negative manner similar to our observations with WT HCN1. FIG. 22 shows that this was indeed the case. Currents recorded from oocytes co-injected with 50 nL WT HCN2 and 50 nL HCN1-AAA cRNA were significantly smaller than those expressed in oocytes injected with 50 nL WT HCN2 alone or 50 nL WT HCN2+50 nL dH₂O after the same incubation period (FIG. 22A-C). In fact, the extents of suppression by HCN1-AAA were similar for both WT HCN1 and HCN2 for all other ratios studied (FIG. 20; total cRNA injected=25 ng). Taken together, these results indicate that the two isoforms were able to coassemble with equivalent efficacy. Similar to HCN1, steady-state activation parameters, reversal potential, and gating kinetics of the non-suppressed HCN2 currents were not changed by HCN1-AAA coexpression (p>0.05; FIG. 22D-F).

### Engineered HCN1 Channels Exhibit Channel Activation Shifted in Positive and Negative Directions.

Modulation of HCN channel gating properties by protein engineering also was accomplished. FIGS. 23A and B show that the charge-neutralizing substitutions E235A produced a significant depolarizing shift in steady-state channel activation (V_{1/2}-59.2.+−.1.5 mV, n=7; p<0.05) with and insignificant change in the slope factor (k=12.3.+−.0.9 mV, n=7; p>0.05). Consistent with an electrostatic role of residue 235, the charge-reversed mutation E235R shifted the steady-state activation curve even more positively (56.4.+−.0.5 mV, n=3; p<0.05). Neither the slope factor (7.9.+−.0.8 mV, n=3; p>0.05) nor P_{o,min} (17.0%+1.9%, n=3; p>0.05) was affected by E235R (p>0.05; FIGS. 23A and C). We next investigated whether the S4 serine variant at position 253 underlies the distinctive activation profile of HCN1 channels by multiple substitutions (FIG. 18). Replacing S253 with alanine (S253A) produced parallel hyperpolarizing shifts in the steady-state activation relationship and the voltage-dependence of gating kinetics while slowing both activation and deactivation (FIG. 23D). S253A, however, did not alter Pₒ min. Despite the opposite charges of S253K and S253E, both substitutions shifted the steady-state I-V relationship in the same hyperpolarizing direction. Taken collectively, this shows that the activation threshold of HCN channel activity can be modulated (FIG. 23) as well as the endogenous expressed current amplitude (FIGS. 18-22).

Several embodiments of the invention has been described in detail with reference to preferred embodiments thereof. However, it will be appreciated that those skilled in the art, upon consideration of the disclosure, may make modification and improvements within the spirit and scope of the invention.

What is claimed is:

1. A method of assaying whether an agent affects heart rate which comprises:
(a) contacting a cardiac cell of a heart with an effective amount of a compound to cause a repetitive heart rate;
(b) measuring the heart rate after step (a);
(c) providing the heart with an agent to be assayed for its affects on heart rate;
(d) measuring the heart rate after step (c); and
(e) comparing the difference between step (b) and step (d), thereby determining whether the agent affects heart rate.

2. The method of claim 1, wherein the heart is mammalian.

3. The method of claim 1, wherein the cardiac cell comprises a cardiac myocyte.

4. The method of claim 1, wherein the compound comprises a nucleic acid which encodes an HCN channel.

5. The method of claim 4, wherein the HCN channel comprises HCN1.

6. The method of claim 4, wherein the HCN channel comprises HCN2.

7. The method of claim 1, wherein the step of contacting is selected from the group consisting of one or more of the following: topical application, injection, liposome application, viral-mediated contact, contacting the cell with the nucleic acid, and coculturing the cell with the nucleic acid.

8. The method of claim 7, wherein administration of contacting is selected from the group consisting of one or more of the following: topical administration, adenovirus infection, viral-mediated infection, liposome-mediated transfer, topical application to the cell, and catheterization.

9. A method of assaying whether an agent affects heart rate which comprises:
(a) isolating cardiac myocytes from a heart;
(b) measuring the beating rate of the cardiac myocytes after step (a);
(c) contacting a set of the cardiac myocytes form step (a) with an agent to be assayed for its effects on heart rate;
(d) measuring the heart rate after step (c); and
(e) comparing the measurements from step (b) and step (d), thereby determining whether the agent affects heart rate.

10. A method of assaying whether an agent affects the membrane potential of a cell which comprises:
(a) contacting the cell with a sufficient amount of a compound capable of lessening the negativity of the membrane potential of the cell;
(b) measuring the membrane potential of the cell after step (a);
(c) providing the cell with the an agent to be assayed for its effects on the membrane potential of a cell;
(d) measuring the membrane potential of the cell after step (c); and
(e) comparing the difference between the measurements from step (b) and step (d), thereby determining whether the agent affects the membrane potential of the cell.

11. A method of assaying whether an agent affects the activation of a cell which comprises:
(a) contacting the cell with a sufficient amount of a compound to activate the cell;
(b) measuring the voltage required to activate the cell after step (a);
(c) providing the cell with an agent to be assayed for its effects on the activation of the cell;
(d) measuring the voltage required to activate the cell after step (c); and
(e) comparing the difference between the measurements from step (b) and step (d), thereby determining whether the agent affects the activation of the cell.

12. A method of assaying whether an agent affects the contraction of a cell which comprises:
(a) contacting a cell with an effective amount of a compound to contract the cell;
(b) measuring the level of contraction of the cell after step (a);
(c) contacting the cell with the agent to be assayed for its effects on contraction of the cell;
(d) measuring the level of contraction of the cell after step (c); and
(e) comparing the difference between the measurements from step (b) and step (d), thereby determining whether the agent affects the contraction of the cell.

13. A vector which comprises a compound which encodes an ion channel gene.

14. The vector of claim 13, wherein the vector is selected from the group consisting of a virus, a plasmid and a cosmid.

15. The vector of claim 13, wherein the vector is an adenovirus.

16. The vector of claim 13, wherein the compound comprises a nucleic acid which encodes an HCN channel.

17. The vector of claim 16, wherein the HCN channel comprises HCN1.

18. The vector of claim 16, wherein the HCN channel comprises HCN2.

19. A method of assaying whether an agent affects heart rate which comprises:
(a) contacting a cardiac cell of a heart with an effective amount of a compound to cause a sustainable heart rate;
(b) measuring the heart rate after step (a);
(c) providing the heart with an agent to be assayed for its affects on heart rate;
(d) measuring the heart rate after step (c); and
(e) comparing the difference between step (b) and step (d), thereby determining whether the agent affects heart rate.

20. A method of assaying whether an agent affects heart rate which comprises:
(a) disaggregating cardiac myocytes from a heart;
(b) measuring the beating rate of the cardiac myocytes after step (a);
(c) contacting a set of the cardiac myocytes form step (a) with an agent to be assayed for its effects on heart rate;
(d) measuring the heart rate after step (c); and
(e) comparing the measurements from step (b) and step (d), thereby determining whether the agent affects heart rate.


SEQUENCE LISTING (25 sequences)

SEQ ID NO: 1 - DNA, 843 bp, Homo sapiens
cccggcctttttttttcctttttcgactagctgcaacccagagggagaaggcggtaaacccgccttaagactgagaaaaccgcagtccagaaaggctcccgagttcgtagatcccaaaacaagtttactggactcattaactttaacaaatgacaaagacacgcctcctccacctaactcgcccaactcgcagaagctcagagggctggttcctgctctgccctcgagggcaccgatccccaccctcgggttaacagatccgccctcccggctgtccagcaacagagctcccggcgcttcgcacccaatcacagcccggtcccgcctgcagcccgcccagtgccgggtcccggggtttggaaccacccctattgccttttctccgcgtggccccgcctgcacccaggcccgagcctgggctgcctaacttcccccttcgctccgccctcgagccaatcaacagcctctaatctcctctggccccgcctgcaagcccgccccggcccagtcacaggcttggttcgcccaggccccacccccggcccgccccgccgtcggtgcgcggcggtagggaaggcgcctcccgcagtcgctcggaactgccgacccgagtgcttcccgcagagggctggtggtgggagcggagtgggtcgggcggggccgagccgggccgtgggccgtgtgggggccgggcggcggccgggccggcggacggcgggatgggctgcaccgtgagcgccgaggacaaggcggcggccgagcgctctaagatgatcgacaagaacctgcgggaggacggagagaaggcggcgcgggaggtgaagttgctgctgttgggtgagg

SEQ ID NO: 2 - DNA, 63 bp, Homo sapiens
gccctctgttccaggtgctggggagtcagggaagagcaccatcgtcaagcagatgaagtaagt

SEQ ID NO: 3 - DNA, 162 bp, Homo sapiens
gtcctggctatcaggatcatccacgaggatggctactccgaggaggaatgccggcagtaccgggcggttgtctacagcaacaccatccagtccatcatggccattgtcaaagccatgggcaacctgcagatcgactttgccgacccctccagagcggtatgt

SEQ ID NO: 4 - DNA, 181 bp, Homo sapiens
gccactgtgcccaggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctagtgagt

SEQ ID NO: 5 - DNA, 149 bp, Homo sapiens
ccccccatccccagctacctgaacgacctggagcgtattgcacagagtgactacatccccacacagcaagatgtgctacggacccgcgtaaagaccacggggatcgtggagacacacttcaccttcaaggacctacacttcaagtgagc

SEQ ID NO: 6 - DNA, 142 bp, Homo sapiens
ctgcaggatgtttgatgtgggtggtcagcggtctgagcggaagaagtggatccactgctttgagggcgtcacagccatcatcttctgcgtagccttgagcgcctatgacttggtgctagctgaggacgaggagatggtgaga

SEQ ID NO: 7 - DNA, 174 bp, Homo sapiens
tattctacccccagaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggtgtgg

SEQ ID NO: 8 - DNA, 235 bp, Homo sapiens
tttctctcccccaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctgaggggcagcggggcctggcgggatggtgatc

SEQ ID NO: 9 - DNA, 1036 bp, Homo sapiens
gctttcccccacctccagggccaccgccgactttgtaccccccaacccctgaggaagatgggggcaagaagatcacgctccccgcctgttcccccgccgcttttctcctctttcctctctttgttctcagctccccctgtcccctcagctccagacgtaggggaggggttgccacaggcctccctgtttgaagcctgcccttgtctgagatgctggtaatggccatggtacccccttctgggcatctgttctggtttttaaccattgtcttgttctgtgatgaggggaggggggcacatgctgagtctcccaaggctgcgtctggaggggcccctgcttctccagcctggacccccagctttgcccaacaccagcccctgccccagcccaagtccaaatgtttacagggagcctcctgcccagtcccccaaccccagccgctcggaggccccaaaggaaaaagcacaagaagcgtgagacgccaccattcctggaaaccacagtccacctgctcattctcgtagctttttaaaaaaatgaaagtaaaggaaaaaaaaaaaactgaaatctagaaaactttttagagaaaaactatttaaaactgtcagatcctgaccagcaagcccccccccagccccccttccaagtgactccgtgccttgagtgtgtctgcgtgtttacacccgtccctctgctggccgcccccgtgcgagcggcacccctgccctgccctccacagaattgggttccaagggctgttccagacaactgccaacgtcactgagggccctgccccagcggccctggccccaggctctattaacctaaaatgtagctccctagcgctaacctaggaaccgccgctgcctgctggggggccacgcccctcatgcccttgtcccaggcccggggccttcagcgttgaacacttccttgcttttttcacatgttttatggaattgttcacctggtttgaaataataaaatgtagaaagaaaaaaaataccgagaactgatgggtattctctcccagggg

SEQ ID NO: 10 - protein, 355 aa, Homo sapiens
MGCTVSAEDKAAAERSKMIDKNLREDGEKAAREVKLLLLGAGESGKSTIVKQMKIIHEDGYSEEECRQYRAVVYSNTIQSIMAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 11 - protein, 23 aa, Homo sapiens
SYALFKAMSHMLCIGYGAQAPVS

SEQ ID NO: 12 - protein, 23 aa, Homo sapiens
SFALFKAMSHMLCIGYGRQAPES

SEQ ID NO: 13 - protein, 23 aa, Homo sapiens
SHALFKAMSHMLCIGYGQQAPVG

SEQ ID NO: 14 - protein, 23 aa, Homo sapiens
SYALFKAMSHMLCIGYGRQAPVG

SEQ ID NO: 15 - protein, 23 aa, Homo sapiens
TWALFKALSHMLCIGYGKFPPQS

SEQ ID NO: 16 - protein, 23 aa, Homo sapiens
PDAFWWAVVTMTTVGYGDMTPVG

SEQ ID NO: 17 - protein, 23 aa, Homo sapiens
PRALWWSVETATTVGYGDLYPVT

SEQ ID NO: 18 - protein, 34 aa, Homo sapiens
EVYKTARALRIVRFTKILSLLRLLRLSRLTRYTH

SEQ ID NO: 19 - protein, 34 aa, Homo sapiens
EVYKATRALRIVRFTKILSLLRLLRLSRLIRYIH

SEQ ID NO: 20 - protein, 34 aa, Homo sapiens
EVYKTARALRIVRFTKILSLLRLLRLSRLIRYMH

SEQ ID NO: 21 - protein, 34 aa, Homo sapiens
EVYKTARALRIVRFTKILSLLRLLRLSRLIRYIH

SEQ ID NO: 22 - protein, 31 aa, Homo sapiens
EVSRALKILRFAKLLSLLRLLRLSRLMRFVS

SEQ ID NO: 23 - protein, 32 aa, Homo sapiens
ELGFRILSMLRLWRLRRVSSLFARLEKDIRFN

SEQ ID NO: 24 - protein, 22 aa, Homo sapiens
AILRVIRLVRVFRIFKLSRHSK

SEQ ID NO: 25 - protein, 22 aa, Homo sapiens
GLLKTARLLRLVRVARKLDRYS